(19) United States
(12) Patent Application Publication
(10) Pub. No.: US 2021/0163485 A1
(43) Pub. Date: Jun. 3, 2021

(54) Heterocyclic Compounds for the Treatment of Disease

(71) Applicant: Oppilan Pharma Ltd., Cambridge (GB)

(72) Inventors: Raju MOHAN, Encinitas, CA (US); John NUSS, San Diego, CA (US); Jason HARRIS, Carlsbad, CA (US)

(21) Appl. No.: 16/613,534

(22) PCT Filed: May 16, 2018
(86) PCT No.: PCT/IB2018/000600

§ 371 Date: Nov. 14, 2019

Related U.S. Application Data
(60) Provisional application No. 62/507,702, filed on May 17, 2017.

(51) Int. Cl. C07D 487/04; A61P 25/00; C07D 471/04

(52) U.S. Cl. CPC C07D 487/04; C07D 471/04; A61P 25/00

(57) ABSTRACT

Described herein are heterocyclic compounds, compositions, and methods for their use for treatment of disease.

### CROSS-REFERENCE

This application claims benefit of U.S. Provisional Application No. 62/507,702, filed on May 17, 2017, which is herein incorporated by reference in its entirety.

### BACKGROUND OF THE INVENTION

The sphingosine-1-phosphate (SIP) receptors are a class of G protein-coupled receptors that are targets of the lipid signalling molecule sphingosine-1-phosphate. Sphingosine-1-phosphate (SIP) is a bioactive sphingolipid that has been demonstrated to induce many cellular processes, including those that result in platelet aggregation, cell proliferation, cell morphology, tumor-cell invasion, endothelial cell chemotaxis and angiogenesis, cytoskeletal re-arrangements in many cell types to regulate immune cell trafficking, vascular homeostasis and cell communication in the central nervous system (CNS) and in peripheral organ systems. SIP can bind with members of the endothelial cell differentiation gene family (EDG receptors) of plasma membrane-localized G protein-coupled receptors. To date, five members of this family have been identified as SIP receptors in different cell types, S1P1 (EDG-1), S1P2 (EDG-5), S1P3 (EDG-3), S1P4 (EDG-6) and S1P5 (EDG-8). SIP receptor modulators are compounds which signal as agonists or antagomists at one or more SIP receptors. Since SIP mediates a wide variety of cellular responses, SIP receptor modulators are promising targets for a variety of therapeutic indications.

### SUMMARY OF THE INVENTION

Described herein are compounds of Formula (I), (Ia), (Ib), (Ic), (Id), (II), (IIa), (IIb), (IIc), (IId), (III), (IIIa), (IIIb), (IIIc), (IIId), or (IIIe), pharmaceutical compositions that include such compounds, and methods of use thereof, for modulating the SIP receptor. In one aspect is the administration of a therapeutically effective amount of at least one SIP receptor modulator described herein to a mammal in the treatment of diseases, disorders or conditions that would benefit from SIP receptor modulation.

In one aspect, provided herein is a compound of Formula (I), or a pharmaceutically acceptable salt or solvate thereof:

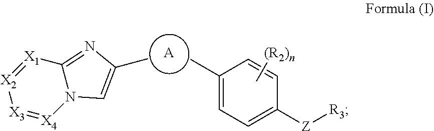

wherein:
X₁ is N; X₂, X₃, and X₄ are each CR₁; or
X₂ is N; X₁, X₃, and X₄ are each CR₁; or
X₃ is N; X₁, X₂, and X₄ are each CR₁; or
X₄ is N; X₁, X₂, and X₃ are each CR₁;

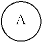

is selected from

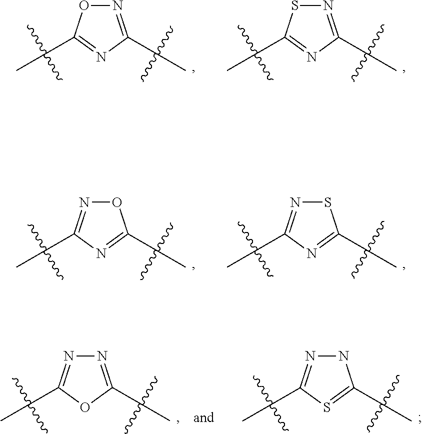

Z is —O—, —S—, —N(R₄)—, or —CH₂—;
each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₁₂, —N(R₁₃)N(R₁₁)S(O)₂R₁₅, —C(O)R₁₄, —C(O)OR₁₀, —C(S)OR₁₀, —C(O)SR₁₀, —C(O)N(R₁₁)R₁₂, —C(S)N(R₁₁)R₁₂, —C(O)N(R₁₁)S(O)₂R₁₅, —C(S)N(R₁₁)S(O)₂R₁₅, —C(O)N(R₁₃)N(R₁₁)R₁₂, —C(S)N(R₁₃)N(R₁₁)R₁₂, and —C(O)N(R₁₃)N(R₁₁)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl;
R₄ is hydrogen or optionally substituted C₁-C₆alkyl;
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring; and
n is 0-4.

In another aspect, provided herein is a compound of Formula (II), or a pharmaceutically acceptable salt or solvate thereof:

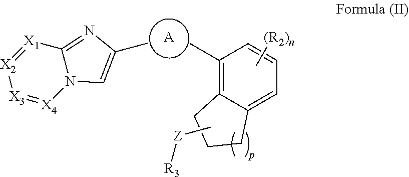

wherein:
X₁ is N; X₂, X₃, and X₄ are each CR₁; or
X₂ is N; X₁, X₃, and X₄ are each CR₁; or
X₃ is N; X₁, X₂, and X₄ are each CR₁; or
X₄ is N; X₁, X₂, and X₃ are each CR₁;

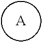

is selected from

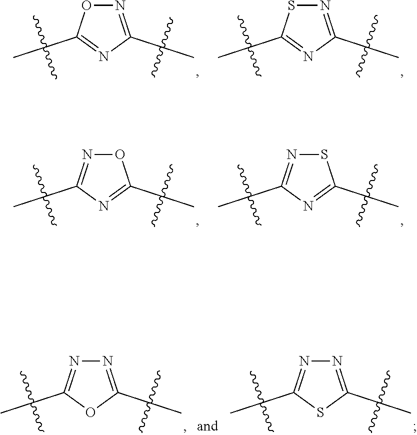 [SMILES: *c1noc(*)n1 ; *c1nsc(*)n1 ; *c1noc(*)n1 ; *c1nsc(*)n1 ; *c1nnc(*)o1 ; *c1nnc(*)s1]

Z is —O—, —S—, —N(R₄)—, or —CH₂—;
each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₁₂, —N(R₁₃)N(R₁₁)S(O)₂R₁₅, —C(O)R₁₄, —C(O)OR₁₀, —C(S)OR₁₀, —C(O)SR₁₀, —C(O)N(R₁₁)R₁₂, —C(S)N(R₁₁)R₁₂, —C(O)N(R₁₁)S(O)₂R₁₅, —C(S)N(R₁₁)S(O)₂R₁₅, —C(O)N(R₁₃)N(R₁₁)R₁₂, —C(S)N(R₁₃)N(R₁₁)R₁₂, and —C(O)N(R₁₃)N(R₁₁)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl;
R₄ is hydrogen or optionally substituted C₁-C₆alkyl;
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring;
n is 0-3, and
p is 1 or 2.

In one embodiment is a compound of Formula (II), or a pharmaceutically acceptable salt or solvate thereof, wherein p is 1. In another embodiment is a compound of Formula (II), or a pharmaceutically acceptable salt or solvate thereof, wherein p is 2. In another embodiment is a compound of Formula (I) or (II), or a pharmaceutically acceptable salt or solvate thereof, wherein Z is —O—. In another embodiment is a compound of Formula (I) or (II), or a pharmaceutically acceptable salt or solvate thereof, wherein Z is —N(H)—. In another embodiment is a compound of Formula (I) or (II), or a pharmaceutically acceptable salt or solvate thereof, wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)N(alkyl)₂. In another embodiment is a compound of Formula (I) or (II), or a pharmaceutically acceptable salt or solvate thereof, wherein R₃ is C₁-C₆alkyl substituted with one or two —OH.

In another aspect, provided herein is a compound of Formula (III), or a pharmaceutically acceptable salt or solvate thereof:

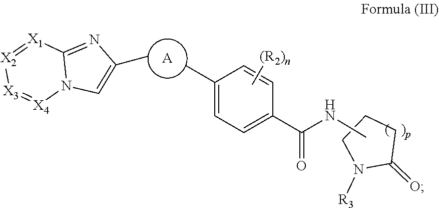

wherein:
X₁, X₂, X₃, and X₄ are each CR₁; or
X₁ is N; X₂, X₃, and X₄ are each CR₁; or
X₂ is N; X₁, X₃, and X₄ are each CR₁; or
X₃ is N; X₁, X₂, and X₄ are each CR₁; or
X₄ is N; X₁, X₂, and X₃ are each CR₁;

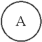

is selected from

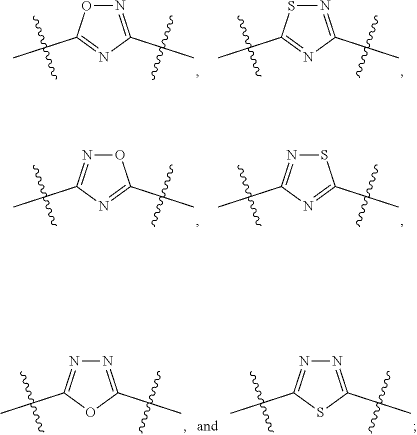 [SMILES: *c1noc(*)n1 ; *c1nsc(*)n1 ; *c1noc(*)n1 ; *c1nsc(*)n1 ; *c1nnc(*)o1 ; *c1nnc(*)s1]

each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₂, —N(R₁₃)N(R₁₁)S(O)₂R₁₅, —C(O)R₁₄, —C(O)OR₁₀, —C(S)OR₁₀, —C(O)SR₁₀, —C(O)N(R₁₁)R₁₂, —C(S)N(R₁₁)R₁₂, —C(O)N(R₁₁)S(O)₂R₁₅, —C(S)N(R₁₁)S(O)₂R₁₅, —C(O)N(R₁₃)N(R₁₁)R₁₂, —C(S)N(R₁₃)N(R₁₁)R₁₂, and —C(O)N(R₁₃)N(R₁₁)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, and optionally substituted —(C₁-C₂alkylene)-(heteroaryl);
R₄ is hydrogen or optionally substituted C₁-C₆alkyl;
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring;
n is 0-4; and
p is 1 or 2.

In one embodiment is a compound of Formula (III), or a pharmaceutically acceptable salt or solvate thereof, wherein p is 1. In another embodiment is a compound of Formula (III), or a pharmaceutically acceptable salt or solvate thereof, wherein p is 2. In another embodiment is a compound of Formula (III), or a pharmaceutically acceptable salt or solvate thereof, wherein R₃ is hydrogen or C₁-C₆alkyl. In another embodiment is a compound of Formula (III), or a pharmaceutically acceptable salt or solvate thereof, wherein R₃ is hydrogen. In another embodiment is a compound of Formula (III), or a pharmaceutically acceptable salt or solvate thereof, wherein X₁, X₂, X₃, and X₄ are each CR₁. In another embodiment is a compound of Formula (I), (II), or (III), or a pharmaceutically acceptable salt or solvate thereof, wherein X₁ is N; X₂, X₃, and X₄ are each CR₁. In another embodiment is a compound of Formula (I), (II), or (III), or a pharmaceutically acceptable salt or solvate thereof, wherein X₂ is N; and X₁, X₃, and X₄ are each CR₁. In another embodiment is a compound of Formula (I), (II), or (III), or a pharmaceutically acceptable salt or solvate thereof, wherein X₃ is N; and X₁, X₂, and X₄ are each CR₁. In another embodiment is a compound of Formula (I), (II), or (III), or a pharmaceutically acceptable salt or solvate thereof, wherein X₄ is N; and X₁, X₂, and X₃ are each CR₁. In another embodiment is a compound of Formula (I), (II), or (III), or a pharmaceutically acceptable salt or solvate thereof, wherein each R₁ is independently selected from the group consisting of hydrogen, halogen, optionally substituted C₁-C₆alkyl, —CF₃, —OR₁₀, —N(R₁₁)R₁₂, —C(O)R₁₄, —C(O)OR₁₀, and —C(O)N(R₁₁)R₁₂. In another embodiment is a compound of Formula (I), (II), or (III), or a pharmaceutically acceptable salt or solvate thereof, wherein each R₁ is independently selected from the group consisting of hydrogen, halogen, and —CF₃. In another embodiment is a compound of Formula (I), (II), or (III), or a pharmaceutically acceptable salt or solvate thereof, wherein

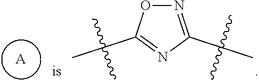

In another embodiment is a compound of Formula (I), (II), or (III), or a pharmaceutically acceptable salt or solvate thereof, wherein

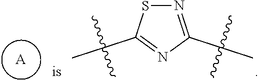

In another embodiment is a compound of Formula (I), (II), or (III), or a pharmaceutically acceptable salt or solvate thereof, wherein

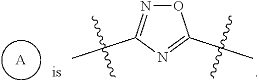

In another embodiment is a compound of Formula (I), (II), or (III), or a pharmaceutically acceptable salt or solvate thereof, wherein

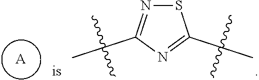

In another embodiment is a compound of Formula (I), (II), or (III), or a pharmaceutically acceptable salt or solvate thereof, wherein

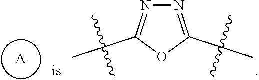

In another embodiment is a compound of Formula (I), (II), or (III), or a pharmaceutically acceptable salt or solvate thereof, wherein

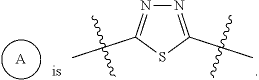

In another embodiment is a compound of Formula (I), (II), or (III), or a pharmaceutically acceptable salt or solvate thereof, wherein each R₂ is independently selected from the group consisting of halogen, optionally substituted C₁-C₆alkyl, —OR₂₀, and —N(R₂₁)R₂₂. In another embodiment is a compound of Formula (I), (II), or (III), or a pharmaceutically acceptable salt or solvate thereof, wherein each R₂ is independently selected from the group consisting of halogen and C₁-C₆alkyl. In another embodiment is a compound of Formula (I), (II), or (III), or a pharmaceutically acceptable salt or solvate thereof, wherein n is 0. In another embodiment is a compound of Formula (I), (II), or (III), or a pharmaceutically acceptable salt or solvate thereof, wherein n is 1. In another embodiment is a compound of Formula (I), (II), or (III), or a pharmaceutically acceptable salt or solvate thereof, wherein n is 2.

Any combination of the groups described above or below for the various variables is contemplated herein. Throughout the specification, groups and substituents thereof are chosen by one skilled in the field to provide stable moieties and compounds.

In another aspect, provided herein is a pharmaceutical composition comprising a compound of Formula (I), (II), or (III), or a pharmaceutically acceptable salt or solvate thereof, and a pharmaceutically acceptable diluent, excipient or binder. In one embodiment, the pharmaceutical composition comprising the compound of Formula (I), (II), or (III), or a pharmaceutically acceptable salt or solvate thereof, is formulated for a route of administration selected from oral administration, parenteral administration, buccal administration, nasal administration, topical administration, or rectal administration.

In another aspect is a method of treating a disease, disorder or condition in a mammal that would benefit from S1P receptor modulation comprising administering to the mammal a therapeutically effective amount of a compound of Formula (I), (II), or (III), or a pharmaceutically acceptable salt or solvate thereof.

In another embodiment is a method of treating a disease, disorder or condition in a mammal that would benefit from S1P receptor modulation comprising administering to the mammal a therapeutically effective amount of a compound of Formula (I), (II), or (III), or a pharmaceutically acceptable salt or solvate thereof; wherein the disease, disorder or condition in a mammal is selected from multiple sclerosis, ulcerative colitis, and Crohn's disease. In another embodiment is a method of treating a disease, disorder or condition in a mammal that would benefit from S1P receptor modulation comprising administering to the mammal a therapeutically effective amount of a compound of Formula (I), (II), or (III), or a pharmaceutically acceptable salt or solvate thereof; wherein the disease, disorder or condition in a mammal is multiple sclerosis. In another embodiment is a method of treating a disease, disorder or condition in a mammal that would benefit from S1P receptor modulation comprising administering to the mammal a therapeutically effective amount of a compound of Formula (I), (II), or (III), or a pharmaceutically acceptable salt or solvate thereof; wherein the disease, disorder or condition in a mammal is ulcerative colitis. In another embodiment is a method of treating a disease, disorder or condition in a mammal that would benefit from SP receptor modulation comprising administering to the mammal a therapeutically effective amount of a compound of Formula (I), (II), or (III), or a pharmaceutically acceptable salt or solvate thereof; wherein the disease, disorder or condition in a mammal is Crohn's disease.

In a further embodiment is a method of treating a disease, disorder or condition in a mammal that would benefit from S1P receptor modulation comprising administering to the mammal a therapeutically effective amount of a compound of Formula (I), (II), or (III), or a pharmaceutically acceptable salt or solvate thereof; wherein the disease, disorder or condition in a mammal is rejection of transplanted organs or tissue; graft-versus-host diseases brought about by transplantation; autoimmune syndromes including rheumatoid arthritis, multiple sclerosis, myasthenia gravis; pollen allergies; type I diabetes; prevention of psoriasis; Crohn's disease; ulcerative colitis, acute respiratory distress syndrome; adult respiratory distress syndrome; influenza; post-infectious autoimmune diseases including rheumatic fever and post-infectious glomerulonephritis; and metastasis of carcinoma.

In another embodiment is the use of a compound of (I), (II), or (III), in the manufacture of a medicament for the treatment of a disease, disorder, or condition that would benefit from S1P receptor modulation. In another embodiment is the use of a S1P receptor modulator in the manufacture of a medicament for use in the treatment of a disease, disorder or condition in a mammal, wherein the disease, disorder or condition in a mammal is rejection of transplanted organs or tissue; graft-versus-host diseases brought about by transplantation; autoimmune syndromes including rheumatoid arthritis, multiple sclerosis, myasthenia gravis; pollen allergies; type I diabetes; prevention of psoriasis; Crohn's disease; ulcerative colitis, acute respiratory distress syndrome; adult respiratory distress syndrome; influenza; post-infectious autoimmune diseases including rheumatic fever and post-infectious glomerulonephritis; and metastasis of carcinoma.

In another aspect is a method of modulating S1P receptor activity comprising contacting the S1P receptor, or portion thereof, with a compound of Formula (I), (II), or (III), or a pharmaceutically acceptable salt or solvate thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

The sphingosine-1-phosphate receptors regulate fundamental biological processes such as cell proliferation, angiogenesis, migration, cytoskeleton organization, endothelial cell chemotaxis, immune cell trafficking and mitogenesis. Sphingosine-1-phosphate receptors are also involved in immune-modulation and directly involved in suppression of innate immune responses from T cells. Sphingosine-1-phosphate (S1P) receptors are divided into five subtypes: S1PR1, S1PR2, S1PR3, S1PR4 and S1PR5. They are expressed in a wide variety of tissues, with each subtype exhibiting different cell specificity, although they are found at their highest density on leukocytes.

Described herein are compounds of Formula (I), (Ia), (Ib), (Ic), (Id), (II), (IIa), (IIb), (IIc), (IId), (III), (IIIa), (IIIb), (IIIc), (IIId), or (IIIe), pharmaceutical compositions that include such compounds, and methods of use thereof, for modulating the S1P receptor. In some embodiments described herein are compounds of Formula (I), (Ia), (Ib), (Ic), (Id), (II), (IIa), (IIb), (IIc), (IId), (III), (IIIa), (IIIb), (IIIc), (IIId), or (IIIe), pharmaceutical compositions that include such compounds, and methods of use thereof, for selectiving modulating S1P receptor subtypes. In some embodiments described herein are compounds of Formula (I), (Ia), (Ib), (Ic), (Id), (II), (IIa), (IIb), (IIc), (IId), (III), (IIIa), (IIb), (IIIc), (IIId), or (IIIe), pharmaceutical compositions that include such compounds, and methods of use thereof, for selectiving modulating two S1P receptor subtypyes. In some embodiments described herein are compounds of Formula (I), (Ia), (Ib), (Ic), (Id), (II), (IIa), (IIb), (IIc), (IId), (III), (IIa), (IIb), (IIIc), (Id), or (IIIe), pharmaceutical compositions that include such compounds, and methods of use thereof, for selectiving modulating a single S1P receptor subtype. In some embodiments described herein are compounds of Formula (I), (Ia), (Ib), (Ic), (Id), (II), (IIa), (IIb), (IIc), (IId), (III), (IIIa), (IIIb), (IIIc), (IIId), or (IIIe), pharmaceutical compositions that include such compounds, and methods of use thereof, for selectiving modulating S1P receptor subtype 1. In some embodiments described herein are compounds of Formula (I), (Ia), (Ib), (Ic), (Id), (II), (Ia), (IIb), (IIc), (IId), (III), (IIIa), (IIb), (IIIc), (IIId), or (IIIe), pharmaceutical compositions that include such compounds, and methods of use thereof, for selectiving modulating S1P receptor subtype 2. In some embodiments described herein are compounds of Formula (I), (Ia), (Ib), (Ic), (Id), (II), (IIa), (Ib), (IIc), (IId), (III), (IIIa), (IIb), (IIIc), (IIId), or (IIIe), pharmaceutical compositions that include such compounds, and methods of use thereof, for selectiving modulating S1P receptor subtype 3. In some embodiments described herein are compounds of Formula (I), (Ia), (Ib), (Ic), (Id), (II), (IIa), (IIb), (IIc), (IId), (III), (IIIa), (IIIb), (IIIc), (IId), or (IIIe), pharmaceutical compositions that include such compounds, and methods of use thereof, for selectiving modulating S1P receptor subtype 4. In some embodiments described herein are compounds of Formula (I), (Ia), (Ib), (Ic), (Id), (II), (IIa), (IIb), (IIc), (IId), (III), (IIIa), (IIIb), (IIIc), (IIId), or (IIIe), pharmaceutical compositions that include such compounds, and methods of use thereof, for selectiving modulating S1P receptor subtype 5.

In another aspect is the administration of at least one SP receptor modulator described herein to a mammal in the treatment of diseases, disorders or conditions that would benefit from S1P receptor modulation. In some embodiments is the administration of at least one S1P receptor modulator described herein to a mammal in the treatment of diseases, disorders or conditions that would benefit from the selective modulatation of S1P receptor subtypes. In some embodiments is the administration of at least one S1P receptor modulator described herein to a mammal in the treatment of diseases, disorders or conditions that would benefit from the selective modulatation of two S1P receptor subtypes. In some embodiments is the administration of at least one S1P receptor modulator described herein to a mammal in the treatment of diseases, disorders or conditions that would benefit from the selective modulatation of one S1P receptor subtype. In some embodiments is the administration of at least one S1P receptor modulator described herein to a mammal in the treatment of diseases, disorders or conditions that would benefit from the selective modulatation of S1P receptor subtype 1. In some embodiments is the administration of at least one S1P receptor modulator described herein to a mammal in the treatment of diseases, disorders or conditions that would benefit from the selective modulatation of S1P receptor subtype 2. In some embodiments is the administration of at least one S1P receptor modulator described herein to a mammal in the treatment of diseases, disorders or conditions that would benefit from the selective modulatation of S1P receptor subtype 3. In some embodiments is the administration of at least one S1P receptor modulator described herein to a mammal in the treatment of diseases, disorders or conditions that would benefit from the selective modulatation of S1P receptor subtype 4. In some embodiments is the administration of at least one S1P receptor modulator described herein to a mammal in the treatment of diseases, disorders or conditions that would benefit from the selective modulatation of S1P receptor subtype 5.

In some embodiments, is a method of modulating S1P receptor activity comprising contacting S1P receptor, or portion thereof, with a compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof. In some embodiments, the compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, is an S1P receptor agonist. In some embodiments, the compound of Formula (I), (Ia), (Ib), (Ic), (Id), (II), (Ia), (IIb), (IIc), (IId), (III), (IIIa), (IIIb), (IIIc), (IIId), or (IIIe), or a pharmaceutically acceptable salt or solvate thereof, is an S1P receptor subtype 1 agonist. In some embodiments, the compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, is an S1P receptor subtype 2 agonist. In some embodiments, the compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, is an S1P receptor subtype 3 agonist. In some embodiments, the compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, is an S1P receptor subtype 4 agonist. In some embodiments, the compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, is an S1P receptor subtype 5 agonist. In some embodiments, the compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, is an S1P receptor partial agonist. In some embodiments, the compound of Formula (I), (Ia), (Ib), (Ic), (Id), (II), (Ia), (IIb), (IIc), (IId), (III), (IIIa), (IIIb), (IIIc), (IIId), or (IIIe), or a pharmaceutically acceptable salt or solvate thereof, is an S1P receptor subtype 1 partial agonist. In some embodiments, the compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, is an S1P receptor subtype 2 partial agonist. In some embodiments, the compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, is an S1P receptor subtype 3 partial agonist. In some embodiments, the compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, is an S1P receptor subtype 4 partial agonist. In some embodiments, the compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, is an S1P receptor subtype 5 partial agonist. In some embodiments, the compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, is an S1P receptor antagonist. In some embodiments, the compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, is an S1P receptor subtype 1 antagonist. In some embodiments, the compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, is an S1P receptor subtype 2 antagonist. In some embodiments, the compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, is an S1P receptor subtype 3 antagonist. In some embodiments, the compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, is an S1P receptor subtype 4 antagonist. In some embodiments, the compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, is an S1P receptor subtype 5 antagonist.

### Compounds

In some embodiments, provided herein is a compound of Formula (I), or a pharmaceutically acceptable salt or solvate thereof:

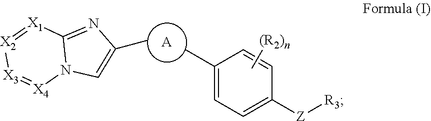

wherein:
X₁ is N; X₂, X₃, and X₄ are each CR₁; or
X₂ is N; X₁, X₃, and X₄ are each CR₁; or
X₃ is N; X₁, X₂, and X₄ are each CR₁; or
X₄ is N; X₁, X₂, and X₃ are each CR₁;

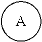

is selected from

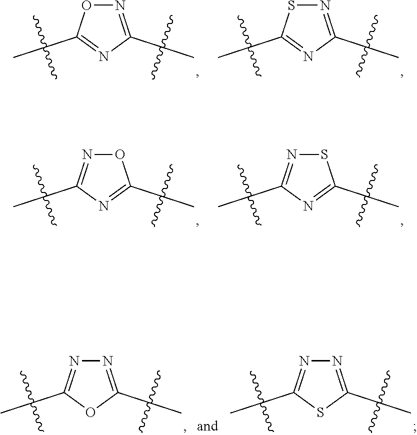 [SMILES: *c1noc(*)n1 ; *c1nsc(*)n1 ; *c1noc(*)n1 ; *c1nsc(*)n1 ; *c1nnc(*)o1 ; *c1nnc(*)s1]

Z is —O—, —S—, —N(R₄)—, or —CH₂—;
each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₁₂, —N(R₁₃)N(R₁₁)S(O)₂R₁₅, —C(O)R₁₄, —C(O)OR₁₀, —C(S)OR₁₀, —C(O)SR₁₀, —C(O)N(R₁₁)R₁₂, —C(S)N(R₁₁)R₁₂, —C(O)N(R₁₁)S(O)₂R₁₅, —C(S)N(R₁₁)S(O)₂R₁₅, —C(O)N(R₁₃)N(R₁₁)R₁₂, —C(S)N(R₁₃)N(R₁₁)R₁₂, and —C(O)N(R₁₃)N(R₁₁)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₈cycloalkyl, C₁-C₆alkoxy, C₂-C₉heterocycloalkyl, phenyl, and heteroaryl;
R₄ is hydrogen or optionally substituted C₁-C₆alkyl;
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring; and
n is 0-4.

In another embodiment is a compound of Formula (I) wherein X₁ is N; and X₂, X₃, and X₄ are each CR₁. In another embodiment is a compound of Formula (I) wherein X₁ is N; and X₂, X₃, and X₄ are each CR₁; and each R₁ is independently selected from the group consisting of hydrogen, halogen, optionally substituted C₁-C₆alkyl, —CF₃, —OR₁₀, —N(R₁₁)R₁₂, —C(O)R₁₄, —C(O)OR₁₀, and —C(O)N(R₁₁)R₁₂. In another embodiment is a compound of Formula (I) wherein X₁ is N; and X₂, X₃, and X₄ are each CR; and each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (I) wherein X₁ is N; and X₂, X₃, and X₄ are each CR₁; and each R₁ is independently selected from the group consisting of hydrogen, halogen, and —CF₃. In another embodiment is a compound of Formula (I) wherein each R₁ is independently selected from the group consisting of hydrogen and —CF₃. In another embodiment is a compound of Formula (I) wherein each R₁ is independently selected from the group consisting of hydrogen and halogen.

In another embodiment is a compound of Formula (I) wherein X₂ is N; and X₁, X₃, and X₄ are each CR₁. In another embodiment is a compound of Formula (I) wherein X₂ is N; and X₁, X₃, and X₄ are each CR₁; and each R₁ is independently selected from the group consisting of hydrogen, halogen, optionally substituted C₁-C₆alkyl, —CF₃, —OR₁₀, —N(R₁₁)R₁₂, —C(O)R₁₄, —C(O)OR₁₀, and —C(O)N(R₁₁)R₁₂. In another embodiment is a compound of Formula (I) wherein X₂ is N; and X₁, X₃, and X₄ are each CR; and each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (I) wherein X₂ is N; and X₁, X₃, and X₄ are each CR₁; and each R₁ is independently selected from the group consisting of hydrogen, halogen, and —CF₃.

In another embodiment is a compound of Formula (I) wherein X₃ is N; and X₁, X₂, and X₄ are each CR₁. In another embodiment is a compound of Formula (I) wherein X₃ is N; and X₁, X₂, and X₄ are each CR₁; and each R₁ is independently selected from the group consisting of hydrogen, halogen, optionally substituted C₁-C₆alkyl, —CF₃, —OR₁₀, —N(R₁₁)R₁₂, —C(O)R₁₄, —C(O)OR₁₀, and —C(O)N(R₁)R₂. In another embodiment is a compound of Formula (I) wherein X₃ is N; and X₁, X₂, and X₄ are each CR₁; and each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (I) wherein X₃ is N; and X₁, X₂, and X₄ are each CR₁; and each R₁ is independently selected from the group consisting of hydrogen, halogen, and —CF₃.

In another embodiment is a compound of Formula (I) wherein X₄ is N; and X₁, X₂, and X₃ are each CR₁. In another embodiment is a compound of Formula (I) wherein X₄ is N; and X₁, X₂, and X₃ are each CR; and each R₁ is independently selected from the group consisting of hydrogen, halogen, optionally substituted C₁-C₆alkyl, —CF₃, —OR₁₀, —N(R₁₁)R₁₂, —C(O)R₁₄, —C(O)OR₁₀, and —C(O)N(R₁₁)R₁₂. In another embodiment is a compound of Formula (I) wherein X₄ is N; and X₁, X₂, and X₃ are each CR₁; and each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (I) wherein X₄ is N; and X₁, X₂, and X₃ are each CR₁; and each R₁ is independently selected from the group consisting of hydrogen, halogen, and —CF₃.

In another embodiment is a compound of Formula (I) wherein n is 3 and each R₂ is independently selected from the group consisting of halogen, optionally substituted C₁-C₆alkyl, —OR₂₀, and —N(R₂₁)R₂₂. In another embodiment is a compound of Formula (I) wherein n is 3 and each R₂ is independently selected from the group consisting of halogen and optionally substituted C₁-C₆alkyl. In another embodiment is a compound of Formula (I) wherein n is 2 and each R₂ is independently selected from the group consisting of halogen, optionally substituted C₁-C₆alkyl, —OR₂₀, and —N(R₂₁)R₂₂. In another embodiment is a compound of Formula (I) wherein n is 2 and each R₂ is independently selected from the group consisting of halogen and optionally substituted C₁-C₆alkyl. In another embodiment is a compound of Formula (I) wherein n is 2 and each R₂ is independently halogen. In another embodiment is a compound of Formula (I) wherein n is 2 and each R₂ is —Cl. In another embodiment is a compound of Formula (I) wherein n is 2 and each R₂ is independently optionally substituted C₁-C₆alkyl. In another embodiment is a compound of Formula (I) wherein n is 1 and R₂ is selected from the group consisting of halogen, optionally substituted C₁-C₆alkyl, —OR₂₀, and —N(R₂₁)R₂₂. In another embodiment is a compound of Formula (I) wherein n is 1 and R₂ is selected from the group consisting of halogen and optionally substituted C₁-C₆alkyl. In another embodiment is a compound of Formula (I) wherein n is 1 and R₂ is halogen. In another embodiment is a compound of Formula (I) wherein n is 1 and R₂ is —Cl. In another embodiment is a compound of Formula (I) wherein n is 1 and R₂ is optionally substituted C₁-C₆alkyl. In another embodiment is a compound of Formula (I) wherein n is 0.

In another embodiment is a compound of Formula (I) wherein R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl. In another embodiment is a compound of Formula (I) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl. In another embodiment is a compound of Formula (I) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), and —S(═O)₂N(C₁-C₆alkyl)₂. In another embodiment is a compound of Formula (I) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)NC₁-C₆alkyl)₂. In another embodiment is a compound of Formula (I) wherein R₃ is C₁-C₆alkyl substituted with one, two, or three groups selected from —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)NC₁-C₆alkyl)₂. In another embodiment is a compound of Formula (I) wherein R₃ is C₁-C₆alkyl substituted with one, two, or three groups selected from —NH₂, —NH(C₁-C₆alkyl), —N(C₁-C₆alkyl)₂, and —OH. In another embodiment is a compound of Formula (I) wherein R₃ is C₁-C₆alkyl substituted with one, two, or three groups selected from —NH₂ and —OH. In another embodiment is a compound of Formula (I) wherein R₃ is C₁-C₆alkyl substituted with one or more —OH. In another embodiment is a compound of Formula (I) wherein R₃ is C₁-C₆alkyl substituted with two —OH. In another embodiment is a compound of Formula (I) wherein R₃ is C₁-C₆alkyl substituted with one —OH.

In another embodiment is a compound of Formula (I) wherein

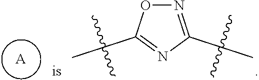

In another embodiment is a compound of Formula (I) wherein

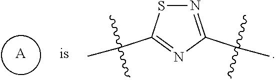

In another embodiment is a compound of Formula (I) wherein

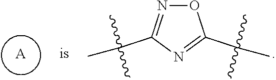

In another embodiment is a compound of Formula (I) wherein

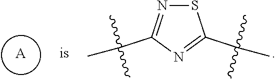

In another embodiment is a compound of Formula (I) wherein

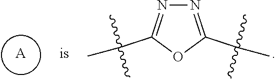

In another embodiment is a compound of Formula (I) wherein

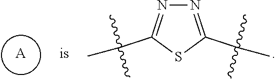

In another embodiment is a compound of Formula (I) wherein Z is —O—, —S—, —N(R₄)—, or —CH₂—. In another embodiment is a compound of Formula (I) wherein Z is —O—. In another embodiment is a compound of Formula (I) wherein Z is —S—. In another embodiment is a compound of Formula (I) wherein Z is —N(R₄)—. In another embodiment is a compound of Formula (I) wherein Z is —N(H)—. In another embodiment is a compound of Formula (I) wherein Z is —N(CH₃)—. In another embodiment is a compound of Formula (I) wherein Z is —CH₂—.

In some embodiments provided herein, the compound of Formula (I) has the structure of Formula (Ia), or a pharmaceutically acceptable salt or solvate thereof:

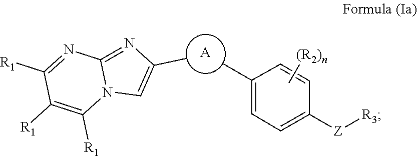

wherein:

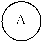

is selected from

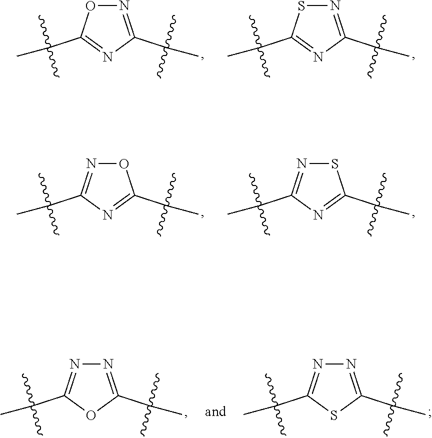 [SMILES: *c1noc(*)n1 ; *c1nsc(*)n1 ; *c1noc(*)n1 ; *c1nsc(*)n1 ; *c1nnc(*)o1 ; *c1nnc(*)s1]

Z is —O—, —S—, —N(R₄)—, or —CH₂—;
each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₁₂, —N(R₁₃)N(R₁₁)S(O)₂R₁₅, —C(O)R₁₄, —C(O)OR₁₀, —C(S)OR₁₀, —C(O)SR₁₀, —C(O)N(R₁₁)R₁₂, —C(S)N(R₁₁)R₁₂, —C(O)N(R₁₁)S(O)₂R₁₅, —C(S)N(R₁₁)S(O)₂R₁₅, —C(O)N(R₁₃)N(R₁₁)R₁₂, —C(S)N(R₁₃)N(R₁₁)R₁₂, and —C(O)N(R₁₃)N(R₁₁)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₈cycloalkyl, C₁-C₆alkoxy, C₂-C₉heterocycloalkyl, phenyl, and heteroaryl;
R₄ is hydrogen or optionally substituted C₁-C₆alkyl;
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring; and
n is 0-4.

In another embodiment is a compound of Formula (Ia) wherein each R₁ is independently selected from the group consisting of hydrogen, halogen, optionally substituted C₁-C₆alkyl, —CF₃, —OR₁₀, —N(R₁₁)R₁₂, —C(O)R₁₄, —C(O)OR₁₀, and —C(O)N(R₁₁)R₁₂. In another embodiment is a compound of Formula (Ia) wherein each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (Ia) wherein each R₁ is independently selected from the group consisting of hydrogen, halogen, and —CF₃. In another embodiment is a compound of Formula (Ia) wherein each R₁ is independently selected from the group consisting of hydrogen and —CF₃. In another embodiment is a compound of Formula (Ia) wherein each R₁ is independently selected from the group consisting of hydrogen and halogen.

In another embodiment is a compound of Formula (Ia) wherein n is 3 and each R₂ is independently selected from the group consisting of halogen, optionally substituted C₁-C₆alkyl, —OR₂₀, and —N(R₂₁)R₂₂. In another embodiment is a compound of Formula (Ia) wherein n is 3 and each R₂ is independently selected from the group consisting of halogen and optionally substituted C₁-C₆alkyl. In another embodiment is a compound of Formula (Ia) wherein n is 2 and each R₂ is independently selected from the group consisting of halogen, optionally substituted C₁-C₆alkyl, —OR₂₀, and —N(R₂₁)R₂₂. In another embodiment is a compound of Formula (Ia) wherein n is 2 and each R₂ is independently selected from the group consisting of halogen and optionally substituted C₁-C₆alkyl. In another embodiment is a compound of Formula (Ia) wherein n is 2 and each R₂ is independently halogen. In another embodiment is a compound of Formula (Ia) wherein n is 2 and each R₂ is —Cl. In another embodiment is a compound of Formula (Ia) wherein n is 2 and each R₂ is independently optionally substituted C₁-C₆alkyl. In another embodiment is a compound of Formula (Ia) wherein n is 1 and R₂ is selected from the group consisting of halogen, optionally substituted C₁-C₆alkyl, —OR₂₀, and —N(R₂₁)R₂₂. In another embodiment is a compound of Formula (Ia) wherein n is 1 and R₂ is selected from the group consisting of halogen and optionally substituted C₁-C₆alkyl. In another embodiment is a compound of Formula (Ia) wherein n is 1 and R₂ is halogen. In another embodiment is a compound of Formula (Ia) wherein n is 1 and R₂ is —Cl. In another embodiment is a compound of Formula (Ia) wherein n is 1 and R₂ is optionally substituted C₁-C₆alkyl. In another embodiment is a compound of Formula (Ia) wherein n is 0.

In another embodiment is a compound of Formula (Ia) wherein R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl. In another embodiment is a compound of Formula (Ia) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl. In another embodiment is a compound of Formula (Ia) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), and —S(═O)₂N(C₁-C₆alkyl)₂. In another embodiment is a compound of Formula (Ia) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)NC₁-C₆alkyl)₂. In another embodiment is a compound of Formula (Ia) wherein R₃ is C₁-C₆alkyl substituted with one, two, or three groups selected from —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)NC₁-C₆alkyl)₂. In another embodiment is a compound of Formula (Ia) wherein R₃ is C₁-C₆alkyl substituted with one, two, or three groups selected from —NH₂, —NH(C₁-C₆alkyl), —N(C₁-C₆alkyl)₂, and —OH. In another embodiment is a compound of Formula (Ia) wherein R₃ is C₁-C₆alkyl substituted with one, two, or three groups selected from —NH₂ and —OH. In another embodiment is a compound of Formula (Ia) wherein R₃ is C₁-C₆alkyl substituted with one or more —OH. In another embodiment is a compound of Formula (Ia) wherein R₃ is C₁-C₆alkyl substituted with two —OH. In another embodiment is a compound of Formula (Ia) wherein R₃ is C₁-C₆alkyl substituted with one —OH.

In another embodiment is a compound of Formula (Ia) wherein

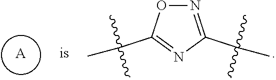

In another embodiment is a compound of Formula (Ia) wherein

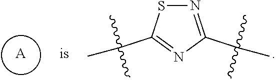

In another embodiment is a compound of Formula (Ia) wherein

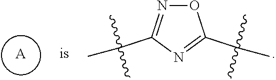

In another embodiment is a compound of Formula (Ia) wherein

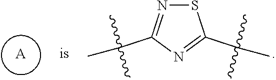

In another embodiment is a compound of Formula (Ia) wherein

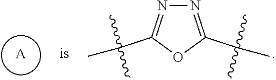

In another embodiment is a compound of Formula (Ia) wherein

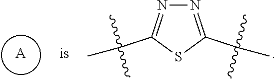

In another embodiment is a compound of Formula (Ia) wherein Z is —O—, —S—, —N(R₄)—, or —CH₂—. In another embodiment is a compound of Formula (Ia) wherein Z is —O—. In another embodiment is a compound of Formula (Ia) wherein Z is —S—. In another embodiment is a compound of Formula (Ia) wherein Z is —N(R₄)—. In another embodiment is a compound of Formula (Ia) wherein Z is —N(H)—. In another embodiment is a compound of Formula (Ia) wherein Z is —N(CH₃)—. In another embodiment is a compound of Formula (Ia) wherein Z is —CH₂—.

In some embodiments provided herein, the compound of Formula (I) has the structure of Formula (Ib), or a pharmaceutically acceptable salt or solvate thereof:

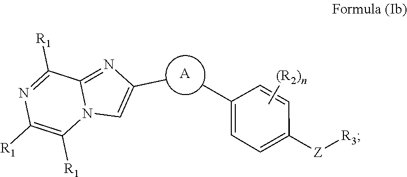

wherein:

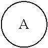

is selected from

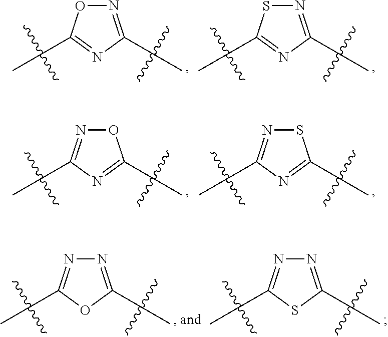 [SMILES: *c1noc(*)n1 ; *c1nsc(*)n1 ; *c1noc(*)n1 ; *c1nsc(*)n1 ; *c1nnc(*)o1 ; *c1nnc(*)s1]

Z is —O—, —S—, —N(R₄)—, or —CH₂—;
each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₁₂, —N(R₁₃)N(R₁₁)S(O)₂R₁₅, —C(O)R₁₄, —C(O)OR₁₀, —C(S)OR₁₀, —C(O)SR₁₀, —C(O)N(R₁₁)R₁₂, —C(S)N(R₁₁)R₁₂, —C(O)N(R₁₁)S(O)₂R₁₅, —C(S)N(R₁₁)S(O)₂R₁₅, —C(O)N(R₁₃)N(R₁₁)R₁₂, —C(S)N(R₁₃)N(R₁₁)R₁₂, and —C(O)N(R₁₃)N(R₁₁)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₈cycloalkyl, C₁-C₆alkoxy, C₂-C₉heterocycloalkyl, phenyl, and heteroaryl;
R₄ is hydrogen or optionally substituted C₁-C₆alkyl;
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring; and
n is 0-4.

In one embodiment is a compound of Formula (Ib) wherein each R₁ is independently selected from the group consisting of hydrogen, halogen, optionally substituted C₁-C₆alkyl, —CF₃, —OR₁₀, —N(R₁₁)R₁₂, —C(O)R₄, —C(O)OR₁₀, and —C(O)N(R₁₁)R₁₂. In another embodiment is a compound of Formula (Ib) wherein each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (Ib) wherein each R₁ is independently selected from the group consisting of hydrogen, halogen, and —CF₃. In another embodiment is a compound of Formula (Ib) wherein each R₁ is independently selected from the group consisting of hydrogen and —CF₃. In another embodiment is a compound of Formula (Ib) wherein each R₁ is independently selected from the group consisting of hydrogen and halogen.

In another embodiment is a compound of Formula (Ib) wherein n is 3 and each R₂ is independently selected from the group consisting of halogen, optionally substituted C₁-C₆alkyl, —OR₂₀, and —N(R₂₁)R₂₂. In another embodiment is a compound of Formula (Ib) wherein n is 3 and each R₂ is independently selected from the group consisting of halogen and optionally substituted C₁-C₆alkyl. In another embodiment is a compound of Formula (Ib) wherein n is 2 and each R₂ is independently selected from the group consisting of halogen, optionally substituted C₁-C₆alkyl, —OR₂₀, and —N(R₂₁)R₂₂. In another embodiment is a compound of Formula (Ib) wherein n is 2 and each R₂ is independently selected from the group consisting of halogen and optionally substituted C₁-C₆alkyl. In another embodiment is a compound of Formula (Ib) wherein n is 2 and each R₂ is independently halogen. In another embodiment is a compound of Formula (Ib) wherein n is 2 and each R₂ is —Cl. In another embodiment is a compound of Formula (Ib) wherein n is 2 and each R₂ is independently optionally substituted C₁-C₆alkyl. In another embodiment is a compound of Formula (Ib) wherein n is 1 and R₂ is selected from the group consisting of halogen, optionally substituted C₁-C₆alkyl, —OR₂₀, and —N(R₂₁)R₂₂. In another embodiment is a compound of Formula (Ib) wherein n is 1 and R₂ is selected from the group consisting of halogen and optionally substituted C₁-C₆alkyl. In another embodiment is a compound of Formula (Ib) wherein n is 1 and R₂ is halogen. In another embodiment is a compound of Formula (Ib) wherein n is 1 and R₂ is —Cl. In another embodiment is a compound of Formula (Ib) wherein n is 1 and R₂ is optionally substituted C₁-C₆alkyl. In another embodiment is a compound of Formula (Ib) wherein n is 0.

In another embodiment is a compound of Formula (Ib) wherein R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl. In another embodiment is a compound of Formula (Ib) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl. In another embodiment is a compound of Formula (Ib) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), and —S(═O)₂N(C₁-C₆alkyl)₂. In another embodiment is a compound of Formula (Ib) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)NC₁-C₆alkyl)₂. In another embodiment is a compound of Formula (Ib) wherein R₃ is C₁-C₆alkyl substituted with one, two, or three groups selected from —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)NC₁-C₆alkyl)₂. In another embodiment is a compound of Formula (Ib) wherein R₃ is C₁-C₆alkyl substituted with one, two, or three groups selected from —NH₂, —NH(C₁-C₆alkyl), —N(C₁-C₆alkyl)₂, and —OH. In another embodiment is a compound of Formula (Ib) wherein R₃ is C₁-C₆alkyl substituted with one, two, or three groups selected from —NH₂ and —OH. In another embodiment is a compound of Formula (Ib) wherein R₃ is C₁-C₆alkyl substituted with one or more —OH. In another embodiment is a compound of Formula (Ib) wherein R₃ is C₁-C₆alkyl substituted with two —OH. In another embodiment is a compound of Formula (Ib) wherein R₃ is C₁-C₆alkyl substituted with one —OH.

In another embodiment is a compound of Formula (Ib) wherein

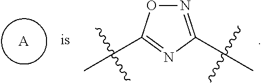

In another embodiment is a compound of Formula (Ib) wherein

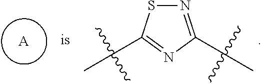

In another embodiment is a compound of Formula (Ib) wherein

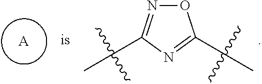

In another embodiment is a compound of Formula (Ib) wherein

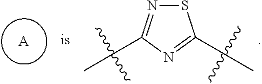

In another embodiment is a compound of Formula (Ib) wherein

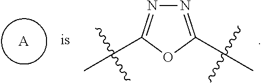

In another embodiment is a compound of Formula (Ib) wherein

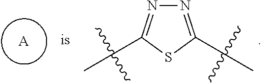

In another embodiment is a compound of Formula (Ib) wherein Z is —O—, —S—, —N(R₄)—, or —CH₂—. In another embodiment is a compound of Formula (Ib) wherein Z is —O—. In another embodiment is a compound of Formula (Ib) wherein Z is —S—. In another embodiment is a compound of Formula (Ib) wherein Z is —N(R₄)—. In another embodiment is a compound of Formula (Ib) wherein Z is —N(H)—. In another embodiment is a compound of Formula (Ib) wherein Z is —N(CH₃)—. In another embodiment is a compound of Formula (Ib) wherein Z is —CH₂—.

In some embodiments provided herein, the compound of Formula (I) has the structure of Formula (Ic), or a pharmaceutically acceptable salt or solvate thereof:

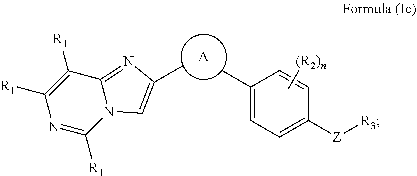

wherein

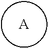

is selected from

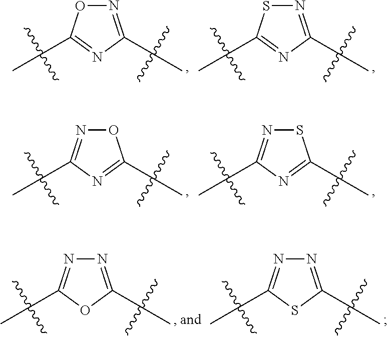 [SMILES: *c1noc(*)n1 ; *c1nsc(*)n1 ; *c1noc(*)n1 ; *c1nsc(*)n1 ; *c1nnc(*)o1 ; *c1nnc(*)s1]

Z is —O—, —S—, —N(R₄)—, or —CH₂—;
each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₁₂, —N(R₁₃)N(R₁₁)S(O)₂R₁₅, —C(O)R₁₄, —C(O)OR₁₀, —C(S)OR₁₀, —C(O)SR₁₀, —C(O)N(R₁₁)R₁₂, —C(S)N(R₁₁)R₁₂, —C(O)N(R₁₁)S(O)₂R₁₅, —C(S)N(R₁₁)S(O)₂R₁₅, —C(O)N(R₁₃)N(R₁₁)R₁₂, —C(S)N(R₁₃)N(R₁₁)R₁₂, and —C(O)N(R₁₃)N(R₁₁)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₈cycloalkyl, C₁-C₆alkoxy, C₂-C₉heterocycloalkyl, phenyl, and heteroaryl;
R₄ is hydrogen or optionally substituted C₁-C₆alkyl;
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring; and
n is 0-4.

In one embodiment is a compound of Formula (Ic) wherein each R₁ is independently selected from the group consisting of hydrogen, halogen, optionally substituted C₁-C₆alkyl, —CF₃, —OR₁₀, —N(R₁₁)R₁₂, —C(O)R₁₄, —C(O)OR₁₀, and —C(O)N(R₁₁)R₁₂. In another embodiment is a compound of Formula (Ic) wherein each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (Ic) wherein each R₁ is independently selected from the group consisting of hydrogen, halogen, and —CF₃. In another embodiment is a compound of Formula (Ic) wherein each R₁ is independently selected from the group consisting of hydrogen and —CF₃. In another embodiment is a compound of Formula (Ic) wherein each R₁ is independently selected from the group consisting of hydrogen and halogen.

In another embodiment is a compound of Formula (Ic) wherein n is 3 and each R₂ is independently selected from the group consisting of halogen, optionally substituted C₁-C₆alkyl, —OR₂₀, and —N(R₂₁)R₂₂. In another embodiment is a compound of Formula (Ic) wherein n is 3 and each R₂ is independently selected from the group consisting of halogen and optionally substituted C₁-C₆alkyl. In another embodiment is a compound of Formula (Ic) wherein n is 2 and each R₂ is independently selected from the group consisting of halogen, optionally substituted C₁-C₆alkyl, —OR₂₀, and —N(R₂₁)R₂₂. In another embodiment is a compound of Formula (Ic) wherein n is 2 and each R₂ is independently selected from the group consisting of halogen and optionally substituted C₁-C₆alkyl. In another embodiment is a compound of Formula (Ic) wherein n is 2 and each R₂ is independently halogen. In another embodiment is a compound of Formula (Ic) wherein n is 2 and each R₂ is —Cl. In another embodiment is a compound of Formula (Ic) wherein n is 2 and each R₂ is independently optionally substituted C₁-C₆alkyl. In another embodiment is a compound of Formula (Ic) wherein n is 1 and R₂ is selected from the group consisting of halogen, optionally substituted C₁-C₆alkyl, —OR₂₀, and —N(R₂₁)R₂₂. In another embodiment is a compound of Formula (Ic) wherein n is 1 and R₂ is selected from the group consisting of halogen and optionally substituted C₁-C₆alkyl. In another embodiment is a compound of Formula (Ic) wherein n is 1 and R₂ is halogen. In another embodiment is a compound of Formula (Ic) wherein n is 1 and R₂ is —Cl. In another embodiment is a compound of Formula (Ic) wherein n is 1 and R₂ is optionally substituted C₁-C₆alkyl. In another embodiment is a compound of Formula (Ic) wherein n is 0.

In another embodiment is a compound of Formula (Ic) wherein R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl. In another embodiment is a compound of Formula (Ic) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl. In another embodiment is a compound of Formula (Ic) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), and —S(═O)₂N(C₁-C₆alkyl)₂. In another embodiment is a compound of Formula (Ic) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)NC₁-C₆alkyl)₂. In another embodiment is a compound of Formula (Ic) wherein R₃ is C₁-C₆alkyl substituted with one, two, or three groups selected from —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)NC₁-C₆alkyl)₂. In another embodiment is a compound of Formula (Ic) wherein R₃ is C₁-C₆alkyl substituted with one, two, or three groups selected from —NH₂, —NH(C₁-C₆alkyl), —N(C₁-C₆alkyl)₂, and —OH. In another embodiment is a compound of Formula (Ic) wherein R₃ is C₁-C₆alkyl substituted with one, two, or three groups selected from —NH₂ and —OH. In another embodiment is a compound of Formula (Ic) wherein R₃ is C₁-C₆alkyl substituted with one or more —OH. In another embodiment is a compound of Formula (Ic) wherein R₃ is C₁-C₆alkyl substituted with two —OH. In another embodiment is a compound of Formula (Ic) wherein R₃ is C₁-C₆alkyl substituted with one —OH.

In another embodiment is a compound of Formula (Ic) wherein

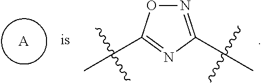

In another embodiment is a compound of Formula (Ic) wherein

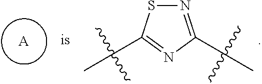

In another embodiment is a compound of Formula (Ic) wherein

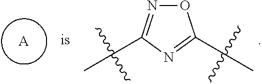

In another embodiment is a compound of Formula (Ic) wherein

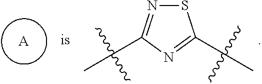

In another embodiment is a compound of Formula (Ic) wherein

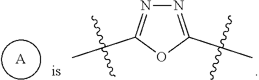

In another embodiment is a compound of Formula (Ic) wherein

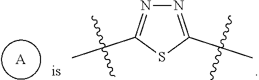

In another embodiment is a compound of Formula (Ic) wherein Z is —O—, —S—, —N(R₄)—, or —CH₂—. In another embodiment is a compound of Formula (Ic) wherein Z is —O—. In another embodiment is a compound of Formula (Ic) wherein Z is —S—. In another embodiment is a compound of Formula (Ic) wherein Z is —N(R₄)—. In another embodiment is a compound of Formula (Ic) wherein Z is —N(H)—. In another embodiment is a compound of Formula (Ic) wherein Z is —N(CH₃)—. In another embodiment is a compound of Formula (Ic) wherein Z is —CH₂—.

In some embodiments provided herein, the compound of Formula (I) has the structure of Formula (Id), or a pharmaceutically acceptable salt or solvate thereof:

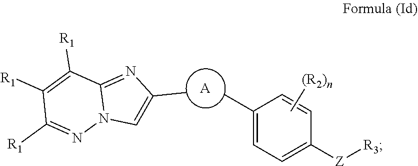

wherein:

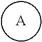

is selected from

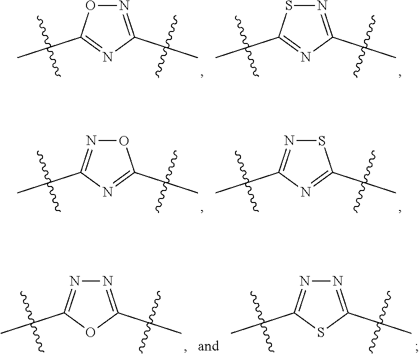 [SMILES: *c1noc(*)n1 ; *c1nsc(*)n1 ; *c1noc(*)n1 ; *c1nsc(*)n1 ; *c1nnc(*)o1 ; *c1nnc(*)s1]

Z is —O—, —S—, —N(R₄)—, or —CH₂—;
each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₁₂, —N(R₁₃)N(R₁₁)S(O)₂R₁₅, —C(O)R₁₄, —C(O)OR₁₀, —C(S)OR₁₀, —C(O)SR₁₀, —C(O)N(R₁₁)R₁₂, —C(S)N(R₁₁)R₁₂, —C(O)N(R₁₁)S(O)₂R₁₅, —C(S)N(R₁₁)S(O)₂R₁₅, —C(O)N(R₁₃)N(R₁₁)R₁₂, —C(S)N(R₁₃)N(R₁₁)R₁₂, and —C(O)N(R₁₃)N(R₁₁)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₈cycloalkyl, C₁-C₆alkoxy, C₂-C₉heterocycloalkyl, phenyl, and heteroaryl;
R₄ is hydrogen or optionally substituted C₁-C₆alkyl;
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃—C cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring; and
n is 0-4.

In one embodiment is a compound of Formula (Id) wherein each R₁ is independently selected from the group consisting of hydrogen, halogen, optionally substituted C₁-C₆alkyl, —CF₃, —OR₁₀, —N(R₁₁)R₁₂, —C(O)R₄, —C(O)OR₁₀, and —C(O)N(R₁₁)R₁₂. In another embodiment is a compound of Formula (Id) wherein each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (Id) wherein each R₁ is independently selected from the group consisting of hydrogen, halogen, and —CF₃. In another embodiment is a compound of Formula (Id) wherein each R₁ is independently selected from the group consisting of hydrogen and —CF₃. In another embodiment is a compound of Formula (Id) wherein each R₁ is independently selected from the group consisting of hydrogen and halogen.

In another embodiment is a compound of Formula (Id) wherein n is 3 and each R₂ is independently selected from the group consisting of halogen, optionally substituted C₁-C₆alkyl, —OR₂₀, and —N(R₂₁)R₂₂. In another embodiment is a compound of Formula (Id) wherein n is 3 and each R₂ is independently selected from the group consisting of halogen and optionally substituted C₁-C₆alkyl. In another embodiment is a compound of Formula (Id) wherein n is 2 and each R₂ is independently selected from the group consisting of halogen, optionally substituted C₁-C₆alkyl, —OR₂₀, and —N(R₂₁)R₂₂. In another embodiment is a compound of Formula (Id) wherein n is 2 and each R₂ is independently selected from the group consisting of halogen and optionally substituted C₁-C₆alkyl. In another embodiment is a compound of Formula (Id) wherein n is 2 and each R₂ is independently halogen. In another embodiment is a compound of Formula (Id) wherein n is 2 and each R₂ is —Cl. In another embodiment is a compound of Formula (Id) wherein n is 2 and each R₂ is independently optionally substituted C₁-C₆alkyl. In another embodiment is a compound of Formula (Id) wherein n is 1 and R₂ is selected from the group consisting of halogen, optionally substituted C₁-C₆alkyl, —OR₂₀, and —N(R₂₁)R₂₂. In another embodiment is a compound of Formula (Id) wherein n is 1 and R₂ is selected from the group consisting of halogen and optionally substituted C₁-C₆alkyl. In another embodiment is a compound of Formula (Id) wherein n is 1 and R₂ is halogen. In another embodiment is a compound of Formula (Id) wherein n is 1 and R₂ is —Cl. In another embodiment is a compound of Formula (Id) wherein n is 1 and R₂ is optionally substituted C₁-C₆alkyl. In another embodiment is a compound of Formula (Id) wherein n is 0.

In another embodiment is a compound of Formula (Id) wherein R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl. In another embodiment is a compound of Formula (Id) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl. In another embodiment is a compound of Formula (Id) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), and —S(═O)₂N(C₁-C₆alkyl)₂. In another embodiment is a compound of Formula (Id) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)NC₁-C₆alkyl)₂. In another embodiment is a compound of Formula (Id) wherein R₃ is C₁-C₆alkyl substituted with one, two, or three groups selected from —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)NC₁-C₆alkyl)₂. In another embodiment is a compound of Formula (Id) wherein R₃ is C₁-C₆alkyl substituted with one, two, or three groups selected from —NH₂, —NH(C₁-C₆alkyl), —N(C₁-C₆alkyl)₂, and —OH. In another embodiment is a compound of Formula (Id) wherein R₃ is C₁-C₆alkyl substituted with one, two, or three groups selected from —NH₂ and —OH. In another embodiment is a compound of Formula (Id) wherein R₃ is C₁-C₆alkyl substituted with one or more —OH. In another embodiment is a compound of Formula (Id) wherein R₃ is C₁-C₆alkyl substituted with two —OH. In another embodiment is a compound of Formula (Id) wherein R₃ is C₁-C₆alkyl substituted with one —OH.

In another embodiment is a compound of Formula (Id) wherein

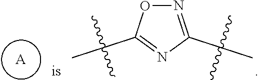

In another embodiment is a compound of Formula (Id) wherein

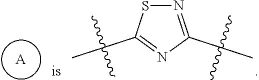

In another embodiment is a compound of Formula (Id) wherein

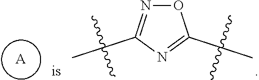

In another embodiment is a compound of Formula (Id) wherein

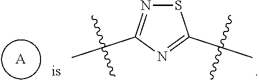

In another embodiment is a compound of Formula (Id) wherein

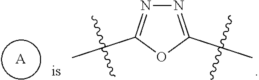

In another embodiment is a compound of Formula (Id) wherein

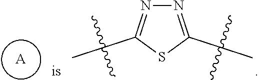

In another embodiment is a compound of Formula (Id) wherein Z is —O—, —S—, —N(R₄)—, or —CH₂—. In another embodiment is a compound of Formula (Id) wherein Z is —O—. In another embodiment is a compound of Formula (Id) wherein Z is —S—. In another embodiment is a compound of Formula (Id) wherein Z is —N(R₄)—. In another embodiment is a compound of Formula (Id) wherein Z is —N(H)—. In another embodiment is a compound of Formula (Id) wherein Z is —N(CH₃)—. In another embodiment is a compound of Formula (Id) wherein Z is —CH₂—.

In some embodiments, provided herein is a compound of Formula (II), or a pharmaceutically acceptable salt or solvate thereof:

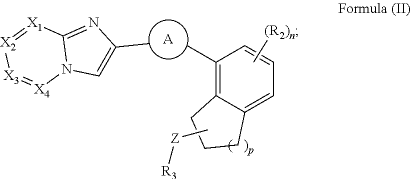

wherein:
X₁ is N; X₂, X₃, and X₄ are each CR₁; or
X₂ is N; X₁, X₃, and X₄ are each CR₁; or
X₃ is N; X₁, X₂, and X₄ are each CR₁; or
X₄ is N; X₁, X₂, and X₃ are each CR₁;

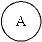

is selected from

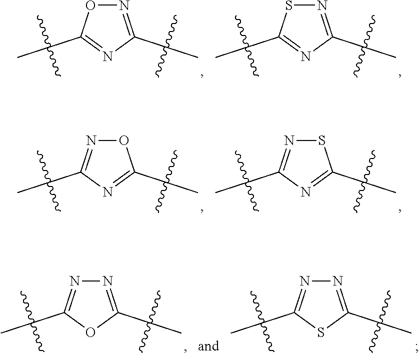 [SMILES: *c1noc(*)n1 ; *c1nsc(*)n1 ; *c1noc(*)n1 ; *c1nsc(*)n1 ; *c1nnc(*)o1 ; *c1nnc(*)s1]

Z is —O—, —S—, —N(R₄)—, or —CH₂—;
each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₁₂, —N(R₁₃)N(R₁₁)S(O)₂R₁₅, —C(O)R₁₄, —C(O)OR₁₀, —C(S)OR₁₀, —C(O)SR₁₀, —C(O)N(R₁₁)R₁₂, —C(S)N(R₁₁)R₁₂, —C(O)N(R₁₁)S(O)₂R₁₅, —C(S)N(R₁₁)S(O)₂R₁₅, —C(O)N(R₁₃)N(R₁₁)R₁₂, —C(S)N(R₁₃)N(R₁₁)R₁₂, and —C(O)N(R₁₃)N(R₁₁)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₈cycloalkyl, C₁-C₆alkoxy, C₂-C₉heterocycloalkyl, phenyl, and heteroaryl;
R₄ is hydrogen or optionally substituted C₁-C₆alkyl;
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring;
n is 0-3, and
p is 1 or 2.

In another embodiment is a compound of Formula (II) wherein X₁ is N; and X₂, X₃, and X₄ are each CR₁. In another embodiment is a compound of Formula (II) wherein X₁ is N; and X₂, X₃, and X₄ are each CR₁; and each R₁ is independently selected from the group consisting of hydrogen, halogen, optionally substituted C₁-C₆alkyl, —CF₃, —OR₁₀, —N(R₁₁)R₁₂, —C(O)R₁₄, —C(O)OR₁₀, and —C(O)N(R₁₁)R₁₂. In another embodiment is a compound of Formula (II) wherein X₁ is N; and X₂, X₃, and X₄ are each CR₁; and each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (II) wherein X₁ is N; and X₂, X₃, and X₄ are each CR₁; and each R₁ is independently selected from the group consisting of hydrogen, halogen, and —CF₃. In another embodiment is a compound of Formula (II) wherein each R₁ is independently selected from the group consisting of hydrogen and —CF₃. In another embodiment is a compound of Formula (II) wherein each R₁ is independently selected from the group consisting of hydrogen and halogen.

In another embodiment is a compound of Formula (II) wherein X₂ is N; and X₁, X₃, and X₄ are each CR₁. In another embodiment is a compound of Formula (II) wherein X₂ is N; and X₁, X₃, and X₄ are each CR₁; and each R₁ is independently selected from the group consisting of hydrogen, halogen, optionally substituted C₁-C₆alkyl, —CF₃, —OR₁₀, —N(R₁₁)R₁₂, —C(O)R₁₄, —C(O)OR₁₀, and —C(O)N(R₁₁)R₁₂. In another embodiment is a compound of Formula (II) wherein X₂ is N; and X₁, X₃, and X₄ are each CR; and each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (II) wherein X₂ is N; and X₁, X₃, and X₄ are each CR₁; and each R₁ is independently selected from the group consisting of hydrogen, halogen, and —CF₃.

In another embodiment is a compound of Formula (II) wherein X₃ is N; and X₁, X₂, and X₄ are each CR₁. In another embodiment is a compound of Formula (II) wherein X₃ is N; and X₁, X₂, and X₄ are each CR₁; and each R₁ is independently selected from the group consisting of hydrogen, halogen, optionally substituted C₁-C₆alkyl, —CF₃, —OR₁₀, —N(R₁₁)R₁₂, —C(O)R₁₄, —C(O)OR₁₀, and —C(O)N(R₁₁)R₁₂. In another embodiment is a compound of Formula (II) wherein X₃ is N; and X₁, X₂, and X₄ are each CR₁; and each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (II) wherein X₃ is N; and X₁, X₂, and X₄ are each CR₁; and each R₁ is independently selected from the group consisting of hydrogen, halogen, and —CF₃.

In another embodiment is a compound of Formula (II) wherein X₄ is N; and X₁, X₂, and X₃ are each CR₁. In another embodiment is a compound of Formula (II) wherein X₄ is N; and X₁, X₂, and X₃ are each CR₁; and each R₁ is independently selected from the group consisting of hydrogen, halogen, optionally substituted C₁-C₆alkyl, —CF₃, —OR₁₀, —N(R₁₁)R₁₂, —C(O)R₁₄, —C(O)OR₁₀, and —C(O)N(R₁₁)R₁₂. In another embodiment is a compound of Formula (II) wherein X₄ is N; and X₁, X₂, and X₃ are each CR; and each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (II) wherein X₄ is N; and X₁, X₂, and X₃ are each CR₁; and each R₁ is independently selected from the group consisting of hydrogen, halogen, and —CF₃.

In another embodiment is a compound of Formula (II) wherein n is 3 and each R₂ is independently selected from the group consisting of halogen, optionally substituted C₁-C₆alkyl, —OR₂₀, and —N(R₂₁)R₂₂. In another embodiment is a compound of Formula (II) wherein n is 3 and each R₂ is independently selected from the group consisting of halogen and optionally substituted C₁-C₆alkyl. In another embodiment is a compound of Formula (II) wherein n is 2 and each R₂ is independently selected from the group consisting of halogen, optionally substituted C₁-C₆alkyl, —OR₂₀, and —N(R₂₁)R₂₂. In another embodiment is a compound of Formula (II) wherein n is 2 and each R₂ is independently selected from the group consisting of halogen and optionally substituted C₁-C₆alkyl. In another embodiment is a compound of Formula (II) wherein n is 2 and each R₂ is independently halogen. In another embodiment is a compound of Formula (II) wherein n is 2 and each R₂ is —Cl. In another embodiment is a compound of Formula (II) wherein n is 2 and each R₂ is independently optionally substituted C₁-C₆alkyl. In another embodiment is a compound of Formula (II) wherein n is 1 and R₂ is selected from the group consisting of halogen, optionally substituted C₁-C₆alkyl, —OR₂₀, and —N(R₂₁)R₂₂. In another embodiment is a compound of Formula (II) wherein n is 1 and R₂ is selected from the group consisting of halogen and optionally substituted C₁-C₆alkyl. In another embodiment is a compound of Formula (II) wherein n is 1 and R₂ is halogen. In another embodiment is a compound of Formula (II) wherein n is 1 and R₂ is —Cl. In another embodiment is a compound of Formula (II) wherein n is 1 and R₂ is optionally substituted C₁-C₆alkyl. In another embodiment is a compound of Formula (II) wherein n is 0.

In another embodiment is a compound of Formula (II) wherein R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl. In another embodiment is a compound of Formula (II) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl. In another embodiment is a compound of Formula (II) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), and —S(═O)₂N(C₁-C₆alkyl)₂. In another embodiment is a compound of Formula (II) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)NC₁-C₆alkyl)₂. In another embodiment is a compound of Formula (II) wherein R₃ is C₁-C₆alkyl substituted with one, two, or three groups selected from —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)NC₁-C₆alkyl)₂. In another embodiment is a compound of Formula (II) wherein R₃ is C₁-C₆alkyl substituted with one, two, or three groups selected from —NH₂, —NH(C₁-C₆alkyl), —N(C₁-C₆alkyl)₂, and —OH. In another embodiment is a compound of Formula (II) wherein R₃ is C₁-C₆alkyl substituted with one, two, or three groups selected from —NH₂ and —OH. In another embodiment is a compound of Formula (II) wherein R₃ is C₁-C₆alkyl substituted with one or more —OH. In another embodiment is a compound of Formula (II) wherein R₃ is C₁-C₆alkyl substituted with two —OH. In another embodiment is a compound of Formula (II) wherein R₃ is C₁-C₆alkyl substituted with one —OH.

In another embodiment is a compound of Formula (II) wherein

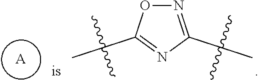

In another embodiment is a compound of Formula (II) wherein

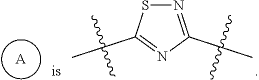

In another embodiment is a compound of Formula (II) wherein

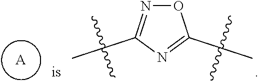

In another embodiment is a compound of Formula (II) wherein

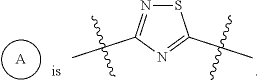

In another embodiment is a compound of Formula (II) wherein

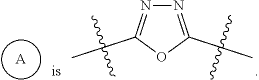

In another embodiment is a compound of Formula (II) wherein

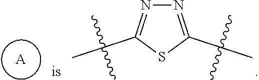

In another embodiment is a compound of Formula (II) wherein Z is —O—, —S—, —N(R₄)—, or —CH₂—. In another embodiment is a compound of Formula (II) wherein Z is —O—. In another embodiment is a compound of Formula (II) wherein Z is —S—. In another embodiment is a compound of Formula (II) wherein Z is —N(R₄)—. In another embodiment is a compound of Formula (II) wherein Z is —N(H)—. In another embodiment is a compound of Formula (II) wherein Z is —N(CH₃)—. In another embodiment is a compound of Formula (II) wherein Z is —CH₂—.

In another embodiment is a compound of Formula (II) wherein p is 1. In another embodiment is a compound of Formula (II) wherein p is 2.

In some embodiments, provided herein is a compound of Formula (IIa), or a pharmaceutically acceptable salt or solvate thereof:

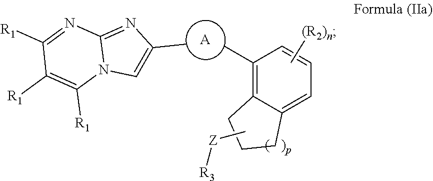

wherein:

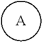

is selected from

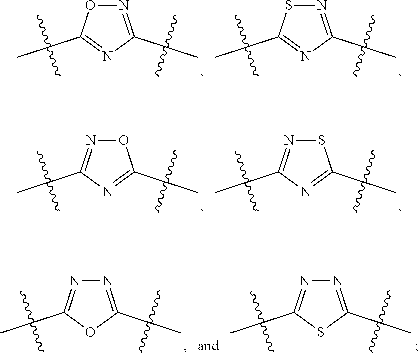 [SMILES: *c1noc(*)n1 ; *c1nsc(*)n1 ; *c1noc(*)n1 ; *c1nsc(*)n1 ; *c1nnc(*)o1 ; *c1nnc(*)s1]

Z is —O—, —S—, —N(R₄)—, or —CH₂—;
each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₁₂, —N(R₁₃)N(R₁₁)S(O)₂R₁₅, —C(O)R₁₄, —C(O)OR₁₀, —C(S)OR₁₀, —C(O)SR₁₀, —C(O)N(R₁₁)R₁₂, —C(S)N(R₁₁)R₁₂, —C(O)N(R₁₁)S(O)₂R₁₅, —C(S)N(R₁₁)S(O)₂R₁₅, —C(O)N(R₁₃)N(R₁₁)R₁₂, —C(S)N(R₁₃)N(R₁₁)R₁₂, and —C(O)N(R₁₃)N(R₁₁)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₈cycloalkyl, C₁-C₆alkoxy, C₂-C₉heterocycloalkyl, phenyl, and heteroaryl;
R₄ is hydrogen or optionally substituted C₁-C₆alkyl;
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring;
n is 0-3, and
p is 1 or 2.

In another embodiment is a compound of Formula (IIa) wherein each R₁ is independently selected from the group consisting of hydrogen, halogen, optionally substituted C₁-C₆alkyl, —CF₃, —OR₁₀, —N(R₁₁)R₁₂, —C(O)R₄, —C(O)OR₁₀, and —C(O)N(R₁₁)R₁₂. In another embodiment is a compound of Formula (IIa) wherein each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (IIa) wherein each R₁ is independently selected from the group consisting of hydrogen, halogen, and —CF₃. In another embodiment is a compound of Formula (IIa) wherein each R₁ is independently selected from the group consisting of hydrogen and —CF₃. In another embodiment is a compound of Formula (IIa) wherein each R₁ is independently selected from the group consisting of hydrogen and halogen.

In another embodiment is a compound of Formula (IIa) wherein n is 3 and each R₂ is independently selected from the group consisting of halogen, optionally substituted C₁-C₆alkyl, —OR₂₀, and —N(R₂₁)R₂₂. In another embodiment is a compound of Formula (IIa) wherein n is 3 and each R₂ is independently selected from the group consisting of halogen and optionally substituted C₁-C₆alkyl. In another embodiment is a compound of Formula (IIa) wherein n is 2 and each R₂ is independently selected from the group consisting of halogen, optionally substituted C₁-C₆alkyl, —OR₂₀, and —N(R₂₁)R₂₂. In another embodiment is a compound of Formula (IIa) wherein n is 2 and each R₂ is independently selected from the group consisting of halogen and optionally substituted C₁-C₆alkyl. In another embodiment is a compound of Formula (IIa) wherein n is 2 and each R₂ is independently halogen. In another embodiment is a compound of Formula (IIa) wherein n is 2 and each R₂ is —Cl. In another embodiment is a compound of Formula (IIa) wherein n is 2 and each R₂ is independently optionally substituted C₁-C₆alkyl. In another embodiment is a compound of Formula (IIa) wherein n is 1 and R₂ is selected from the group consisting of halogen, optionally substituted C₁-C₆alkyl, —OR₂₀, and —N(R₂₁)R₂₂. In another embodiment is a compound of Formula (IIa) wherein n is 1 and R₂ is selected from the group consisting of halogen and optionally substituted C₁-C₆alkyl. In another embodiment is a compound of Formula (IIa) wherein n is 1 and R₂ is halogen. In another embodiment is a compound of Formula (IIa) wherein n is 1 and R₂ is —Cl. In another embodiment is a compound of Formula (IIa) wherein n is 1 and R₂ is optionally substituted C₁-C₆alkyl. In another embodiment is a compound of Formula (IIa) wherein n is 0.

In another embodiment is a compound of Formula (IIa) wherein R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl. In another embodiment is a compound of Formula (IIa) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl. In another embodiment is a compound of Formula (IIa) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), and —S(═O)₂N(C₁-C₆alkyl)₂. In another embodiment is a compound of Formula (IIa) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)NC₁-C₆alkyl)₂. In another embodiment is a compound of Formula (IIa) wherein R₃ is C₁-C₆alkyl substituted with one, two, or three groups selected from —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)NC₁-C₆alkyl)₂. In another embodiment is a compound of Formula (IIa) wherein R₃ is C₁-C₆alkyl substituted with one, two, or three groups selected from —NH₂, —NH(C₁-C₆alkyl), —N(C₁-C₆alkyl)₂, and —OH. In another embodiment is a compound of Formula (IIa) wherein R₃ is C₁-C₆alkyl substituted with one, two, or three groups selected from —NH₂ and —OH. In another embodiment is a compound of Formula (IIa) wherein R₃ is C₁-C₆alkyl substituted with one or more —OH. In another embodiment is a compound of Formula (IIa) wherein R₃ is C₁-C₆alkyl substituted with two —OH. In another embodiment is a compound of Formula (IIa) wherein R₃ is C₁-C₆alkyl substituted with one —OH.

In another embodiment is a compound of Formula (IIa) wherein

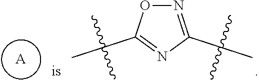

In another embodiment is a compound of Formula (IIa) wherein

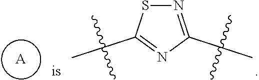

In another embodiment is a compound of Formula (IIa) wherein

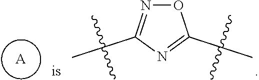

In another embodiment is a compound of Formula (IIa) wherein

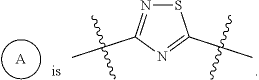

In another embodiment is a compound of Formula (IIa) wherein

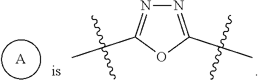

In another embodiment is a compound of Formula (IIa) wherein

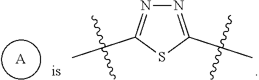

In another embodiment is a compound of Formula (IIa) wherein Z is —O—, —S—, —N(R₄)—, or —CH₂—. In another embodiment is a compound of Formula (IIa) wherein Z is —O—. In another embodiment is a compound of Formula (IIa) wherein Z is —S—. In another embodiment is a compound of Formula (IIa) wherein Z is —N(R₄)—. In another embodiment is a compound of Formula (IIa) wherein Z is —N(H)—. In another embodiment is a compound of Formula (IIa) wherein Z is —N(CH₃)—. In another embodiment is a compound of Formula (IIa) wherein Z is —CH₂—.

In another embodiment is a compound of Formula (IIa) wherein p is 1. In another embodiment is a compound of Formula (IIa) wherein p is 2.

In some embodiments, provided herein is a compound of Formula (IIb), or a pharmaceutically acceptable salt or solvate thereof:

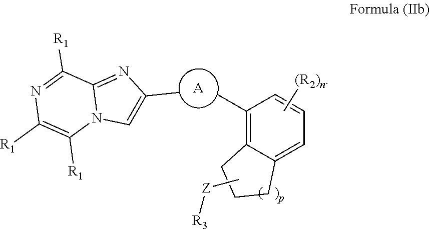

wherein:

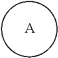

is selected from

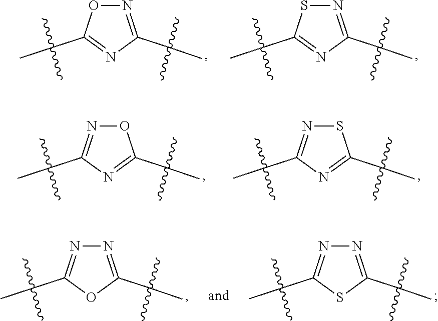 [SMILES: *c1noc(*)n1 ; *c1nsc(*)n1 ; *c1noc(*)n1 ; *c1nsc(*)n1 ; *c1nnc(*)o1 ; *c1nnc(*)s1]

Z is —O—, —S—, —N(R₄)—, or —CH₂—;
each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₁₂, —N(R₁₃)N(R₁₁)S(O)₂R₁₅, —C(O)R₁₄, —C(O)OR₁₀, —C(S)OR₁₀, —C(O)SR₁₀, —C(O)N(R₁₁)R₁₂, —C(S)N(R₁₁)R₁₂, —C(O)N(R₁₁)S(O)₂R₁₅, —C(S)N(R₁₁)S(O)₂R₁₅, —C(O)N(R₁₃)N(R₁₁)Ru, —C(S)N(R₁₃)N(R₁₁)R₁₂, and —C(O)N(R₁₃)N(R₁₁)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₈cycloalkyl, C₁-C₆alkoxy, C₂-C₉heterocycloalkyl, phenyl, and heteroaryl;
R₄ is hydrogen or optionally substituted C₁-C₆alkyl;
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring;
n is 0-3, and
p is 1 or 2.

In another embodiment is a compound of Formula (IIb) wherein each R₁ is independently selected from the group consisting of hydrogen, halogen, optionally substituted C₁-C₆alkyl, —CF₃, —OR₁₀, —N(R₁₁)R₁₂, —C(O)R₄, —C(O)OR₁₀, and —C(O)N(R₁₁)R₁₂. In another embodiment is a compound of Formula (IIb) wherein each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (IIb) wherein each R₁ is independently selected from the group consisting of hydrogen, halogen, and —CF₃. In another embodiment is a compound of Formula (IIb) wherein each R₁ is independently selected from the group consisting of hydrogen and —CF₃. In another embodiment is a compound of Formula (IIb) wherein each R₁ is independently selected from the group consisting of hydrogen and halogen.

In another embodiment is a compound of Formula (IIb) wherein n is 3 and each R₂ is independently selected from the group consisting of halogen, optionally substituted C₁-C₆alkyl, —OR₂₀, and —N(R₂₁)R₂₂. In another embodiment is a compound of Formula (IIb) wherein n is 3 and each R₂ is independently selected from the group consisting of halogen and optionally substituted C₁-C₆alkyl. In another embodiment is a compound of Formula (IIb) wherein n is 2 and each R₂ is independently selected from the group consisting of halogen, optionally substituted C₁-C₆alkyl, —OR₂₀, and —N(R₂₁)R₂₂. In another embodiment is a compound of Formula (IIb) wherein n is 2 and each R₂ is independently selected from the group consisting of halogen and optionally substituted C₁-C₆alkyl. In another embodiment is a compound of Formula (IIb) wherein n is 2 and each R₂ is independently halogen. In another embodiment is a compound of Formula (IIb) wherein n is 2 and each R₂ is —Cl. In another embodiment is a compound of Formula (IIb) wherein n is 2 and each R₂ is independently optionally substituted C₁-C₆alkyl. In another embodiment is a compound of Formula (IIb) wherein n is 1 and R₂ is selected from the group consisting of halogen, optionally substituted C₁-C₆alkyl, —OR₂₀, and —N(R₂₁)R₂₂. In another embodiment is a compound of Formula (IIb) wherein n is 1 and R₂ is selected from the group consisting of halogen and optionally substituted C₁-C₆alkyl. In another embodiment is a compound of Formula (IIb) wherein n is 1 and R₂ is halogen. In another embodiment is a compound of Formula (IIb) wherein n is 1 and R₂ is —Cl. In another embodiment is a compound of Formula (IIb) wherein n is 1 and R₂ is optionally substituted C₁-C₆alkyl. In another embodiment is a compound of Formula (IIb) wherein n is 0.

In another embodiment is a compound of Formula (IIb) wherein R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl. In another embodiment is a compound of Formula (IIb) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl. In another embodiment is a compound of Formula (IIb) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), and —S(═O)₂N(C₁-C₆alkyl)₂. In another embodiment is a compound of Formula (IIb) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)NC₁-C₆alkyl)₂. In another embodiment is a compound of Formula (IIb) wherein R₃ is C₁-C₆alkyl substituted with one, two, or three groups selected from —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)NC₁-C₆alkyl)₂. In another embodiment is a compound of Formula (IIb) wherein R₃ is C₁-C₆alkyl substituted with one, two, or three groups selected from —NH₂, —NH(C₁-C₆alkyl), —N(C₁-C₆alkyl)₂, and —OH. In another embodiment is a compound of Formula (IIb) wherein R₃ is C₁-C₆alkyl substituted with one, two, or three groups selected from —NH₂ and —OH. In another embodiment is a compound of Formula (IIb) wherein R₃ is C₁-C₆alkyl substituted with one or more —OH. In another embodiment is a compound of Formula (IIb) wherein R₃ is C₁-C₆alkyl substituted with two —OH. In another embodiment is a compound of Formula (IIb) wherein R₃ is C₁-C₆alkyl substituted with one —OH.

In another embodiment is a compound of Formula (IIb) wherein

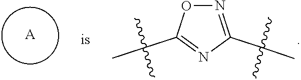

In another embodiment is a compound of Formula (IIb) wherein

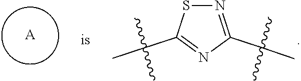

In another embodiment is a compound of Formula (IIb) wherein

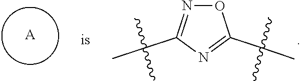

In another embodiment is a compound of Formula (IIb) wherein

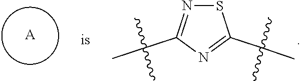

In another embodiment is a compound of Formula (IIb) wherein

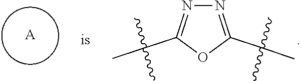

In another embodiment is a compound of Formula (IIb) wherein

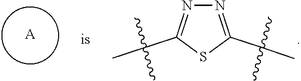

In another embodiment is a compound of Formula (IIb) wherein Z is —O—, —S—, —N(R₄)—, or —CH₂—. In another embodiment is a compound of Formula (IIb) wherein Z is —O—. In another embodiment is a compound of Formula (IIb) wherein Z is —S—. In another embodiment is a compound of Formula (IIb) wherein Z is —N(R₄)—. In another embodiment is a compound of Formula (IIb) wherein Z is —N(H)—. In another embodiment is a compound of Formula (IIb) wherein Z is —N(CH₃)—. In another embodiment is a compound of Formula (IIb) wherein Z is —CH₂—.

In another embodiment is a compound of Formula (IIb) wherein p is 1. In another embodiment is a compound of Formula (IIb) wherein p is 2.

In some embodiments, provided herein is a compound of Formula (IIc), or a pharmaceutically acceptable salt or solvate thereof:

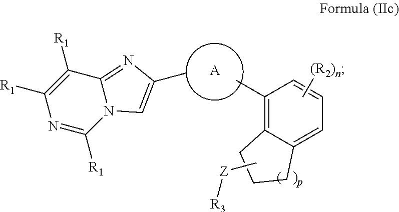

wherein:

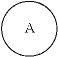

is selected from

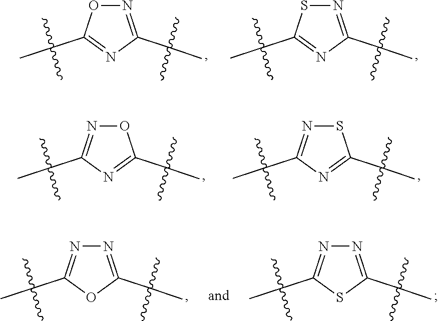 [SMILES: *c1noc(*)n1 ; *c1nsc(*)n1 ; *c1noc(*)n1 ; *c1nsc(*)n1 ; *c1nnc(*)o1 ; *c1nnc(*)s1]

Z is —O—, —S—, —N(R₄)—, or —CH₂—;
each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₁₂, —N(R₁₃)N(R₁₁)S(O)₂R₁₅, —C(O)R₁₄, —C(O)OR₁₀, —C(S)OR₁₀, —C(O)SR₁₀, —C(O)N(R₁₁)R₁₂, —C(S)N(R₁₁)R₁₂, —C(O)N(R₁₁)S(O)₂R₁₅, —C(S)N(R₁₁)S(O)₂R₁₅, —C(O)N(R₁₃)N(R₁₁)R₁₂, —C(S)N(R₁₃)N(R₁₁)R₁₂, and —C(O)N(R₁₃)N(R₁₁)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₈cycloalkyl, C₁-C₆alkoxy, C₂-C₉heterocycloalkyl, phenyl, and heteroaryl;
R₄ is hydrogen or optionally substituted C₁-C₆alkyl;
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring;
n is 0-3, and
p is 1 or 2.

In another embodiment is a compound of Formula (IIc) wherein each R₁ is independently selected from the group consisting of hydrogen, halogen, optionally substituted C₁-C₆alkyl, —CF₃, —OR₁₀, —N(R₁₁)R₁₂, —C(O)R₄, —C(O)OR₁₀, and —C(O)N(R₁₁)R₁₂. In another embodiment is a compound of Formula (IIc) wherein each R₁ is independently selected from the group consisting of hydrogen, halogen, optionally substituted C₁-C₆alkyl, —CF₃, —OR₁₀, and —N(R₁)R₁₂. In another embodiment is a compound of Formula (IIc) wherein each R₁ is independently selected from the group consisting of hydrogen, halogen, and —CF₃. In another embodiment is a compound of Formula (IIc) wherein each R₁ is independently selected from the group consisting of hydrogen and —CF₃. In another embodiment is a compound of Formula (IIc) wherein each R₁ is independently selected from the group consisting of hydrogen and halogen.

In another embodiment is a compound of Formula (IIc) wherein n is 3 and each R₂ is independently selected from the group consisting of halogen, optionally substituted C₁-C₆alkyl, —OR₂₀, and —N(R₂₁)R₂₂. In another embodiment is a compound of Formula (IIc) wherein n is 3 and each R₂ is independently selected from the group consisting of halogen and optionally substituted C₁-C₆alkyl. In another embodiment is a compound of Formula (IIc) wherein n is 2 and each R₂ is independently selected from the group consisting of halogen, optionally substituted C₁-C₆alkyl, —OR₂₀, and —N(R₂₁)R₂₂. In another embodiment is a compound of Formula (IIc) wherein n is 2 and each R₂ is independently selected from the group consisting of halogen and optionally substituted C₁-C₆alkyl. In another embodiment is a compound of Formula (IIc) wherein n is 2 and each R₂ is independently halogen. In another embodiment is a compound of Formula (IIc) wherein n is 2 and each R₂ is —Cl. In another embodiment is a compound of Formula (IIc) wherein n is 2 and each R₂ is independently optionally substituted C₁-C₆alkyl. In another embodiment is a compound of Formula (IIc) wherein n is 1 and R₂ is selected from the group consisting of halogen, optionally substituted C₁-C₆alkyl, —OR₂₀, and —N(R₂₁)R₂₂. In another embodiment is a compound of Formula (IIc) wherein n is 1 and R₂ is selected from the group consisting of halogen and optionally substituted C₁-C₆alkyl. In another embodiment is a compound of Formula (IIc) wherein n is 1 and R₂ is halogen. In another embodiment is a compound of Formula (IIc) wherein n is 1 and R₂ is —Cl. In another embodiment is a compound of Formula (IIc) wherein n is 1 and R₂ is optionally substituted C₁-C₆alkyl. In another embodiment is a compound of Formula (IIc) wherein n is 0.

In another embodiment is a compound of Formula (IIc) wherein R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl. In another embodiment is a compound of Formula (IIc) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl. In another embodiment is a compound of Formula (IIc) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), and —S(═O)₂N(C₁-C₆alkyl)₂. In another embodiment is a compound of Formula (IIc) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)NC₁-C₆alkyl)₂. In another embodiment is a compound of Formula (IIc) wherein R₃ is C₁-C₆alkyl substituted with one, two, or three groups selected from —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)NC₁-C₆alkyl)₂. In another embodiment is a compound of Formula (IIc) wherein R₃ is C₁-C₆alkyl substituted with one, two, or three groups selected from —NH₂, —NH(C₁-C₆alkyl), —N(C₁-C₆alkyl)₂, and —OH. In another embodiment is a compound of Formula (IIc) wherein R₃ is C₁-C₆alkyl substituted with one, two, or three groups selected from —NH₂ and —OH. In another embodiment is a compound of Formula (IIc) wherein R₃ is C₁-C₆alkyl substituted with one or more —OH. In another embodiment is a compound of Formula (IIc) wherein R₃ is C₁-C₆alkyl substituted with two —OH. In another embodiment is a compound of Formula (IIc) wherein R₃ is C₁-C₆alkyl substituted with one —OH.

In another embodiment is a compound of Formula (IIc) wherein

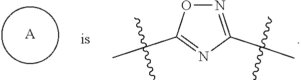

In another embodiment is a compound of Formula (IIc) wherein

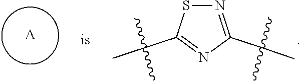

In another embodiment is a compound of Formula (IIc) wherein

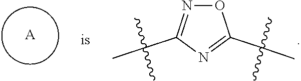

In another embodiment is a compound of Formula (IIc) wherein

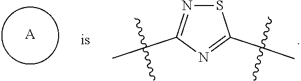

In another embodiment is a compound of Formula (IIc) wherein

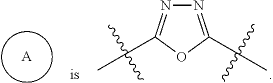

In another embodiment is a compound of Formula (IIc) wherein

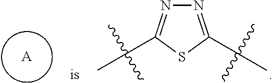

In another embodiment is a compound of Formula (IIc) wherein Z is —O—, —S—, —N(R₄)—, or —CH₂—. In another embodiment is a compound of Formula (IIc) wherein Z is —O—. In another embodiment is a compound of Formula (IIc) wherein Z is —S—. In another embodiment is a compound of Formula (IIc) wherein Z is —N(R₄)—. In another embodiment is a compound of Formula (IIc) wherein Z is —N(H)—. In another embodiment is a compound of Formula (IIc) wherein Z is —N(CH₃)—. In another embodiment is a compound of Formula (IIc) wherein Z is —CH₂—.

In another embodiment is a compound of Formula (IIc) wherein p is 1. In another embodiment is a compound of Formula (IIc) wherein p is 2.

In some embodiments, provided herein is a compound of Formula (Id), or a pharmaceutically acceptable salt or solvate thereof:

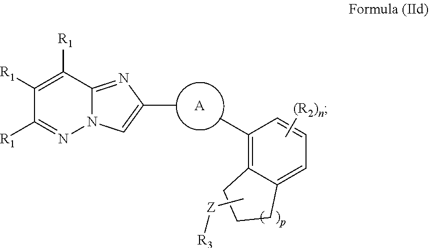

wherein:

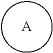

is selected from

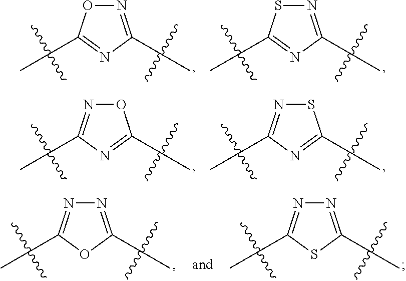 [SMILES: *c1noc(*)n1 ; *c1nsc(*)n1 ; *c1noc(*)n1 ; *c1nsc(*)n1 ; *c1nnc(*)o1 ; *c1nnc(*)s1]

Z is —O—, —S—, —N(R₄)—, or —CH₂—;
each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₁₂, —N(R₁₃)N(R₁₁)S(O)₂R₁₅, —C(O)R₁₄, —C(O)OR₁₀, —C(S)OR₁₀, —C(O)SR₁₀, —C(O)N(R₁₁)R₁₂, —C(S)N(R₁₁)R₁₂, —C(O)N(R₁₁)S(O)₂R₁₅, —C(S)N(R₁₁)S(O)₂R₁₅, —C(O)N(R₁₃)N(R₁₁)R₁₂, —C(S)N(R₁₃)N(R₁₁)R₁₂, and —C(O)N(R₁₃)N(R₁₁)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₈cycloalkyl, C₁-C₆alkoxy, C₂-C₉heterocycloalkyl, phenyl, and heteroaryl;
R₄ is hydrogen or optionally substituted C₁-C₆alkyl;
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring;
n is 0-3, and
p is 1 or 2.

In another embodiment is a compound of Formula (IId) wherein each R₁ is independently selected from the group consisting of hydrogen, halogen, optionally substituted C₁-C₆alkyl, —CF₃, —OR₁₀, —N(R₁₁)R₁₂, —C(O)R₄, —C(O)OR₁₀, and —C(O)N(R₁₁)R₁₂. In another embodiment is a compound of Formula (IId) wherein each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (IId) wherein each R₁ is independently selected from the group consisting of hydrogen, halogen, and —CF₃. In another embodiment is a compound of Formula (IId) wherein each R₁ is independently selected from the group consisting of hydrogen and —CF₃. In another embodiment is a compound of Formula (IId) wherein each R₁ is independently selected from the group consisting of hydrogen and halogen.

In another embodiment is a compound of Formula (IId) wherein n is 3 and each R₂ is independently selected from the group consisting of halogen, optionally substituted C₁-C₆alkyl, —OR₂₀, and —N(R₂₁)R₂₂. In another embodiment is a compound of Formula (IId) wherein n is 3 and each R₂ is independently selected from the group consisting of halogen and optionally substituted C₁-C₆alkyl. In another embodiment is a compound of Formula (IId) wherein n is 2 and each R₂ is independently selected from the group consisting of halogen, optionally substituted C₁-C₆alkyl, —OR₂₀, and —N(R₂₁)R₂₂. In another embodiment is a compound of Formula (IId) wherein n is 2 and each R₂ is independently selected from the group consisting of halogen and optionally substituted C₁-C₆alkyl. In another embodiment is a compound of Formula (IId) wherein n is 2 and each R₂ is independently halogen. In another embodiment is a compound of Formula (IId) wherein n is 2 and each R₂ is —Cl. In another embodiment is a compound of Formula (IId) wherein n is 2 and each R₂ is independently optionally substituted C₁-C₆alkyl. In another embodiment is a compound of Formula (IId) wherein n is 1 and R₂ is selected from the group consisting of halogen, optionally substituted C₁-C₆alkyl, —OR₂₀, and —N(R₂₁)R₂₂. In another embodiment is a compound of Formula (IId) wherein n is 1 and R₂ is selected from the group consisting of halogen and optionally substituted C₁-C₆alkyl. In another embodiment is a compound of Formula (IId) wherein n is 1 and R₂ is halogen. In another embodiment is a compound of Formula (IId) wherein n is 1 and R₂ is —Cl. In another embodiment is a compound of Formula (IId) wherein n is 1 and R₂ is optionally substituted C₁-C₆alkyl. In another embodiment is a compound of Formula (IId) wherein n is 0.

In another embodiment is a compound of Formula (IId) wherein R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl. In another embodiment is a compound of Formula (IId) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl. In another embodiment is a compound of Formula (IId) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), and —S(═O)₂N(C₁-C₆alkyl)₂. In another embodiment is a compound of Formula (IId) wherein R₃ is C₁-C₆alkyl substituted with one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)NC₁-C₆alkyl)₂. In another embodiment is a compound of Formula (IId) wherein R₃ is C₁-C₆alkyl substituted with one, two, or three groups selected from —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)NC₁-C₆alkyl)₂. In another embodiment is a compound of Formula (IId) wherein R₃ is C₁-C₆alkyl substituted with one, two, or three groups selected from —NH₂, —NH(C₁-C₆alkyl), —N(C₁-C₆alkyl)₂, and —OH. In another embodiment is a compound of Formula (IId) wherein R₃ is C₁-C₆alkyl substituted with one, two, or three groups selected from —NH₂ and —OH. In another embodiment is a compound of Formula (IId) wherein R₃ is C₁-C₆alkyl substituted with one or more —OH. In another embodiment is a compound of Formula (IId) wherein R₃ is C₁-C₆alkyl substituted with two —OH. In another embodiment is a compound of Formula (IId) wherein R₃ is C₁-C₆alkyl substituted with one —OH.

In another embodiment is a compound of Formula (IId) wherein

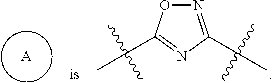

In another embodiment is a compound of Formula (IId) wherein

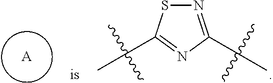

In another embodiment is a compound of Formula (IId) wherein

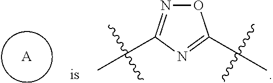

In another embodiment is a compound of Formula (IId) wherein

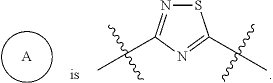

In another embodiment is a compound of Formula (IId) wherein

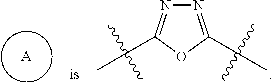

In another embodiment is a compound of Formula (IId) wherein

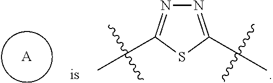

In another embodiment is a compound of Formula (IId) wherein Z is —O—, —S—, —N(R₄)—, or —CH₂—. In another embodiment is a compound of Formula (IId) wherein Z is —O—. In another embodiment is a compound of Formula (IId) wherein Z is —S—. In another embodiment is a compound of Formula (IId) wherein Z is —N(R₄)—. In another embodiment is a compound of Formula (IId) wherein Z is —N(H)—. In another embodiment is a compound of Formula (IId) wherein Z is —N(CH₃)—. In another embodiment is a compound of Formula (IId) wherein Z is —CH₂—.

In another embodiment is a compound of Formula (IId) wherein p is 1. In another embodiment is a compound of Formula (IId) wherein p is 2.

In some embodiments, provided herein is a compound of Formula (III), or a pharmaceutically acceptable salt or solvate thereof:

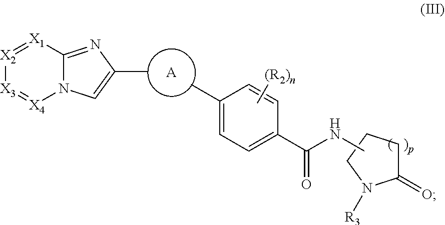

wherein:
X₁, X₂, X₃, and X₄ are each CR₁; or
X₁ is N; X₂, X₃, and X₄ are each CR₁; or
X₂ is N; X₁, X₃, and X₄ are each CR₁; or
X₃ is N; X₁, X₂, and X₄ are each CR₁; or
X₄ is N; X₁, X₂, and X₃ are each CR₁;

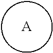

is selected from

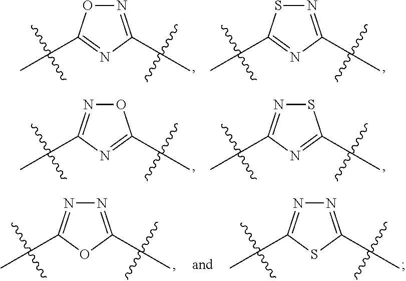 [SMILES: *c1noc(*)n1 ; *c1nsc(*)n1 ; *c1noc(*)n1 ; *c1nsc(*)n1 ; *c1nnc(*)o1 ; *c1nnc(*)s1]

each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₁₂, —N(R₁₃)N(R₁₁)S(O)₂R₁₅, —C(O)R₁₄, —C(O)OR₁₀, —C(S)OR₁₀, —C(O)SR₁₀, —C(O)N(R₁₁)R₁₂, —C(S)N(R₁₁)R₁₂, —C(O)N(R₁₁)S(O)₂R₁₅, —C(S)N(R₁₁)S(O)₂R₁₅, —C(O)N(R₁₃)N(R₁₁)R₁₂, —C(S)N(R₁₃)N(R₁₁)R₁₂, and —C(O)N(R₁₃)N(Rn)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, and optionally substituted —(C₁-C₂alkylene)-(heteroaryl);
R₄ is hydrogen or optionally substituted C₁-C₆alkyl;
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring;
n is 0-4; and
p is 1 or 2.

In another embodiment is a compound of Formula (III) wherein X₁, X₂, X₃, and X₄ are each CR₁. In another embodiment is a compound of Formula (III) wherein X₁, X₂, X₃, and X₄ are each CR₁; and each R₁ is independently selected from the group consisting of hydrogen, halogen, optionally substituted C₁-C₆alkyl, —CF₃, —OR₁₀, —N(R₁₁)R₁₂, —C(O)R₁₄, —C(O)OR₁₀, and —C(O)N(R₁₁)R₁₂. In another embodiment is a compound of Formula (III) wherein X₁, X₂, X₃, and X₄ are each CR₁; and each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (III) wherein X₁, X₂, X₃, and X₄ are each CR₁; and each R₁ is independently selected from the group consisting of hydrogen, halogen, and —CF₃.

In another embodiment is a compound of Formula (III) wherein X₁ is N; and X₂, X₃, and X₄ are each CR₁. In another embodiment is a compound of Formula (III) wherein X₁ is N; and X₂, X₃, and X₄ are each CR₁; and each R₁ is independently selected from the group consisting of hydrogen, halogen, optionally substituted C₁-C₆alkyl, —CF₃, —OR₁₀, —N(R₁₁)R₁₂, —C(O)R₁₄, —C(O)OR₁₀, and —C(O)N(R₁₁)R₁₂. In another embodiment is a compound of Formula (III) wherein X₁ is N; and X₂, X₃, and X₄ are each CR; and each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (III) wherein X₁ is N; and X₂, X₃, and X₄ are each CR₁; and each R₁ is independently selected from the group consisting of hydrogen, halogen, and —CF₃.

In another embodiment is a compound of Formula (III) wherein X₂ is N; and X₁, X₃, and X₄ are each CR₁. In another embodiment is a compound of Formula (III) wherein X₂ is N; and X₁, X₃, and X₄ are each CR₁; and each R₁ is independently selected from the group consisting of hydrogen, halogen, optionally substituted C₁-C₆alkyl, —CF₃, —OR₁₀, —N(R₁₁)R₁₂, —C(O)R₁₄, —C(O)OR₁₀, and —C(O)N(R₁₁)R₁₂. In another embodiment is a compound of Formula (III) wherein X₂ is N; and X₁, X₃, and X₄ are each CR; and each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (III) wherein X₂ is N; and X₁, X₃, and X₄ are each CR₁; and each R₁ is independently selected from the group consisting of hydrogen, halogen, and —CF₃.

In another embodiment is a compound of Formula (III) wherein X₃ is N; and X₁, X₂, and X₄ are each CR₁. In another embodiment is a compound of Formula (III) wherein X₃ is N; and X₁, X₂, and X₄ are each CR₁; and each R₁ is independently selected from the group consisting of hydrogen, halogen, optionally substituted C₁-C₆alkyl, —CF₃, —OR₁₀, —N(R₁₁)R₁₂, —C(O)R₁₄, —C(O)OR₁₀, and —C(O)N(R₁₁)R₁₂. In another embodiment is a compound of Formula (III) wherein X₃ is N; and X₁, X₂, and X₄ are each CR₁; and each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (III) wherein X₃ is N; and X₁, X₂, and X₄ are each CR₁; and each R₁ is independently selected from the group consisting of hydrogen, halogen, and —CF₃.

In another embodiment is a compound of Formula (III) wherein X₄ is N; and X₁, X₂, and X₃ are each CR₁. In another embodiment is a compound of Formula (III) wherein X₄ is N; and X₁, X₂, and X₃ are each CR₁; and each R₁ is independently selected from the group consisting of hydrogen, halogen, optionally substituted C₁-C₆alkyl, —CF₃, —OR₁₀, —N(R₁₁)R₁₂, —C(O)R₁₄, —C(O)OR₁₀, and —C(O)N(R₁₁)R₁₂. In another embodiment is a compound of Formula (III) wherein X₄ is N; and X₁, X₂, and X₃ are each CR; and each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (III) wherein X₄ is N; and X₁, X₂, and X₃ are each CR₁; and each R₁ is independently selected from the group consisting of hydrogen, halogen, and —CF₃.

In another embodiment is a compound of Formula (III) wherein n is 3 and each R₂ is independently selected from the group consisting of halogen, optionally substituted C₁-C₆alkyl, —OR₂₀, and —N(R₂₁)R₂₂. In another embodiment is a compound of Formula (III) wherein n is 3 and each R₂ is independently selected from the group consisting of halogen and optionally substituted C₁-C₆alkyl. In another embodiment is a compound of Formula (III) wherein n is 2 and each R₂ is independently selected from the group consisting of halogen, optionally substituted C₁-C₆alkyl, —OR₂₀, and —N(R₂₁)R₂₂. In another embodiment is a compound of Formula (III) wherein n is 2 and each R₂ is independently selected from the group consisting of halogen and optionally substituted C₁-C₆alkyl. In another embodiment is a compound of Formula (III) wherein n is 1 and R₂ is selected from the group consisting of halogen, optionally substituted C₁-C₆alkyl, —OR₂₀, and —N(R₂₁)R₂₂. In another embodiment is a compound of Formula (III) wherein n is 1 and R₂ is selected from the group consisting of halogen and optionally substituted C₁-C₆alkyl. In another embodiment is a compound of Formula (III) wherein n is 0.

In another embodiment is a compound of Formula (III) wherein R₃ is selected from the group consisting of hydrogen and optionally substituted C₁-C₆alkyl. In another embodiment is a compound of Formula (III) wherein R₃ is hydrogen. In another embodiment is a compound of Formula (III) wherein R₃ is optionally substituted C₁-C₆alkyl. In another embodiment is a compound of Formula (III) wherein R₃ is methyl.

In another embodiment is a compound of Formula (III) wherein

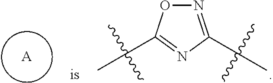

In another embodiment is a compound of Formula (III) wherein

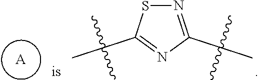

In another embodiment is a compound of Formula (III) wherein

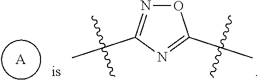

In another embodiment is a compound of Formula (III) wherein

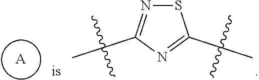

In another embodiment is a compound of Formula (III) wherein

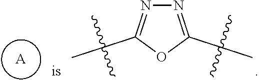

In another embodiment is a compound of Formula (III) wherein

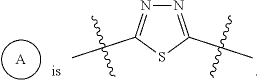

In another embodiment is a compound of Formula (III) wherein p is 1. In another embodiment is a compound of Formula (III) wherein p is 2.

In some embodiments, provided herein is a compound of Formula (IIa), or a pharmaceutically acceptable salt or solvate thereof:

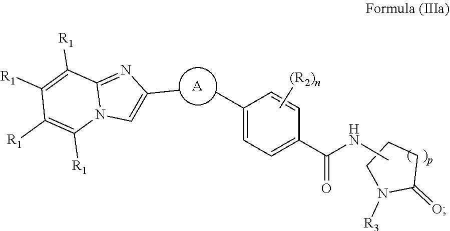

wherein:

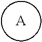

is selected from

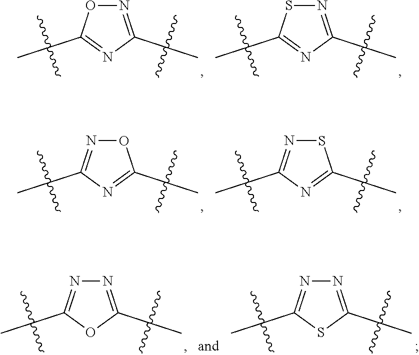 [SMILES: *c1noc(*)n1 ; *c1nsc(*)n1 ; *c1noc(*)n1 ; *c1nsc(*)n1 ; *c1nnc(*)o1 ; *c1nnc(*)s1]

each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₁₂, —N(R₁₃)N(R₁₁)S(O)₂R₁₅, —C(O)R₁₄, —C(O)OR₁₀, —C(S)OR₁₀, —C(O)SR₁₀, —C(O)N(R₁₁)R₁₂, —C(S)N(R₁₁)R₁₂, —C(O)N(R₁₁)S(O)₂R₁₅, —C(S)N(R₁₁)S(O)₂R₁₅, —C(O)N(R₁₃)N(R₁₁)Ru, —C(S)N(R₁₃)N(R₁₁)R₁₂, and —C(O)N(R₁₃)N(R₁₁)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, and optionally substituted —(C₁-C₂alkylene)-(heteroaryl);
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring;
n is 0-4; and
p is 1 or 2.

In another embodiment is a compound of Formula (IIIa) wherein each R₁ is independently selected from the group consisting of hydrogen, halogen, optionally substituted C₁-C₆alkyl, —CF₃, —OR₁₀, —N(R₁₁)R₁₂, —C(O)R₄, —C(O)OR₁₀, and —C(O)N(R₁₁)R₁₂. In another embodiment is a compound of Formula (IIIa) wherein each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (IIIa) wherein each R₁ is independently selected from the group consisting of hydrogen, halogen, and —CF₃. In another embodiment is a compound of Formula (IIIa) wherein each R₁ is independently selected from the group consisting of hydrogen and halogen. In another embodiment is a compound of Formula (IIIa) wherein each R₁ is independently selected from the group consisting of hydrogen and —CF₃.

In another embodiment is a compound of Formula (IIIa) wherein n is 3 and each R₂ is independently selected from the group consisting of halogen, optionally substituted C₁-C₆alkyl, —OR₂₀, and —N(R₂₁)R₂₂. In another embodiment is a compound of Formula (IIIa) wherein n is 3 and each R₂ is independently selected from the group consisting of halogen and optionally substituted C₁-C₆alky. In another embodiment is a compound of Formula (IIIa) wherein n is 2 and each R₂ is independently selected from the group consisting of halogen, optionally substituted C₁-C₆alkyl, —OR₂₀, and —N(R₂₁)R₂₂. In another embodiment is a compound of Formula (IIIa) wherein n is 2 and each R₂ is independently selected from the group consisting of halogen and optionally substituted C₁-C₆alkyl. In another embodiment is a compound of Formula (IIIa) wherein n is 1 and R₂ is selected from the group consisting of halogen, optionally substituted C₁-C₆alkyl, —OR₂₀, and —N(R₂₁)R₂₂. In another embodiment is a compound of Formula (IIIa) wherein n is 1 and R₂ is selected from the group consisting of halogen and optionally substituted C₁-C₆alkyl. In another embodiment is a compound of Formula (IIIa) wherein n is 0.

In another embodiment is a compound of Formula (IIIa) wherein R₃ is selected from the group consisting of hydrogen and optionally substituted C₁-C₆alkyl. In another embodiment is a compound of Formula (IIIa) wherein R₃ is hydrogen. In another embodiment is a compound of Formula (IIIa) wherein R₃ is optionally substituted C₁-C₆alkyl. In another embodiment is a compound of Formula (IIIa) wherein R₃ is methyl.

In another embodiment is a compound of Formula (Ia) wherein

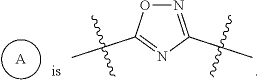

In another embodiment is a compound of Formula (IIIa) wherein

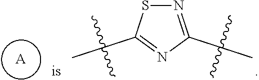

In another embodiment is a compound of Formula (IIIa) wherein

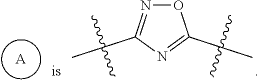

In another embodiment is a compound of Formula (IIIa) wherein

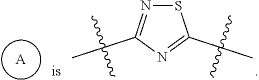

In another embodiment is a compound of Formula (IIIa) wherein

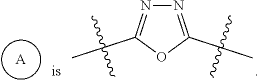

In another embodiment is a compound of Formula (IIIa) wherein

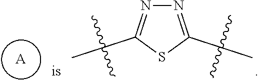

In another embodiment is a compound of Formula (IIIa) wherein p is 1. In another embodiment is a compound of Formula (IIIa) wherein p is 2.

In some embodiments, provided herein is a compound of Formula (IIIb), or a pharmaceutically acceptable salt or solvate thereof:

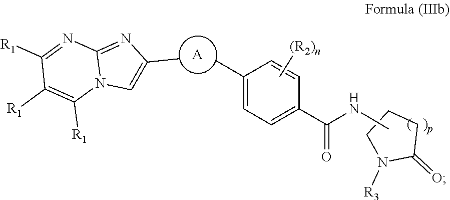

wherein:

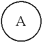

is selected from

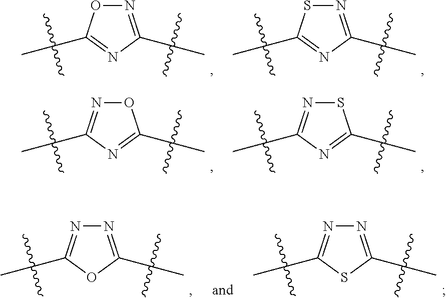 [SMILES: *c1noc(*)n1 ; *c1nsc(*)n1 ; *c1noc(*)n1 ; *c1nsc(*)n1 ; *c1nnc(*)o1 ; *c1nnc(*)s1]

each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₁₂, —N(R₁₃)N(R₁₁)S(O)₂R₁₅, —C(O)R₁₄, —C(O)OR₁₀, —C(S)OR₁₀, —C(O)SR₁₀, —C(O)N(R₁₁)R₁₂, —C(S)N(R₁₁)R₁₂, —C(O)N(R₁₁)S(O)₂R₁₅, —C(S)N(R₁₁)S(O)₂R₁₅, —C(O)N(R₁₃)N(R₁₁)Ru, —C(S)N(R₁₃)N(R₁₁)R₁₂, and —C(O)N(R₁₃)N(R₁₁)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, and optionally substituted —(C₁-C₂alkylene)-(heteroaryl);
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring;
n is 0-4; and
p is 1 or 2.

In another embodiment is a compound of Formula (IIIb) wherein each R₁ is independently selected from the group consisting of hydrogen, halogen, optionally substituted C₁-C₆alkyl, —CF₃, —OR₁₀, —N(R₁₁)R₁₂, —C(O)R₄, —C(O)OR₁₀, and —C(O)N(R₁₁)R₁₂. In another embodiment is a compound of Formula (IIIb) wherein each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (IIIb) wherein each R₁ is independently selected from the group consisting of hydrogen, halogen, and —CF₃. In another embodiment is a compound of Formula (IIIb) wherein each R₁ is independently selected from the group consisting of hydrogen and halogen. In another embodiment is a compound of Formula (IIIb) wherein each R₁ is independently selected from the group consisting of hydrogen and —CF₃.

In another embodiment is a compound of Formula (IIIb) wherein n is 3 and each R₂ is independently selected from the group consisting of halogen, optionally substituted C₁-C₆alkyl, —OR₂₀, and —N(R₂₁)R₂₂. In another embodiment is a compound of Formula (IIIb) wherein n is 3 and each R₂ is independently selected from the group consisting of halogen and optionally substituted C₁-C₆alky. In another embodiment is a compound of Formula (IIIb) wherein n is 2 and each R₂ is independently selected from the group consisting of halogen, optionally substituted C₁-C₆alkyl, —OR₂₀, and —N(R₂₁)R₂₂. In another embodiment is a compound of Formula (IIIb) wherein n is 2 and each R₂ is independently selected from the group consisting of halogen and optionally substituted C₁-C₆alkyl. In another embodiment is a compound of Formula (IIIb) wherein n is 1 and R₂ is selected from the group consisting of halogen, optionally substituted C₁-C₆alkyl, —OR₂₀, and —N(R₂₁)R₂₂. In another embodiment is a compound of Formula (IIIb) wherein n is 1 and R₂ is selected from the group consisting of halogen and optionally substituted C₁-C₆alkyl. In another embodiment is a compound of Formula (IIIb) wherein n is 0.

In another embodiment is a compound of Formula (IIIb) wherein R₃ is selected from the group consisting of hydrogen and optionally substituted C₁-C₆alkyl. In another embodiment is a compound of Formula (IIIb) wherein R₃ is hydrogen. In another embodiment is a compound of Formula (IIIb) wherein R₃ is optionally substituted C₁-C₆alkyl. In another embodiment is a compound of Formula (IIIb) wherein R₃ is methyl.

In another embodiment is a compound of Formula (IIIb) wherein

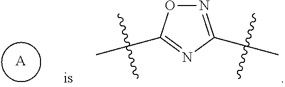

In another embodiment is a compound of Formula (IIIb) wherein

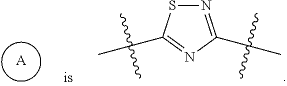

In another embodiment is a compound of Formula (IIIb) wherein

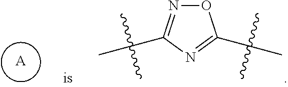

In another embodiment is a compound of Formula (IIIb) wherein

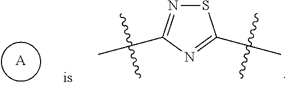

In another embodiment is a compound of Formula (IIIb) wherein

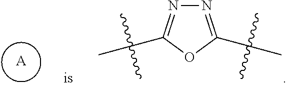

In another embodiment is a compound of Formula (IIIb) wherein

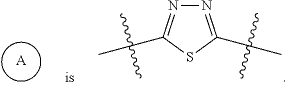

In another embodiment is a compound of Formula (IIIb) wherein p is 1. In another embodiment is a compound of Formula (IIIb) wherein p is 2.

In some embodiments, provided herein is a compound of Formula (IIc), or a pharmaceutically acceptable salt or solvate thereof:

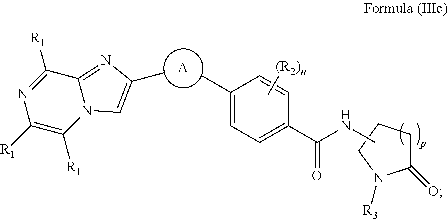

wherein:

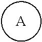

is selected from

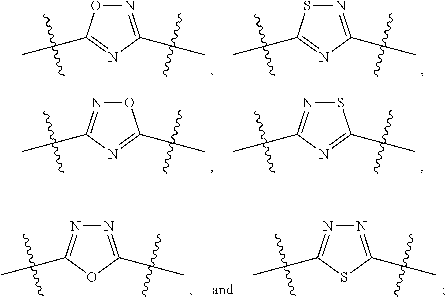 [SMILES: *c1noc(*)n1 ; *c1nsc(*)n1 ; *c1noc(*)n1 ; *c1nsc(*)n1 ; *c1nnc(*)o1 ; *c1nnc(*)s1]

each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₁₂, —N(R₁₃)N(R₁₁)S(O)₂R₁₅, —C(O)R₁₄, —C(O)OR₁₀, —C(S)OR₁₀, —C(O)SR₁₀, —C(O)N(R₁₁)R₁₂, —C(S)N(R₁₁)R₁₂, —C(O)N(R₁₁)S(O)₂R₁₅, —C(S)N(R₁₁)S(O)₂R₁₅, —C(O)N(R₁₃)N(R₁₁)Ru, —C(S)N(R₁₃)N(R₁₁)R₁₂, and —C(O)N(R₁₃)N(R₁₁)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, and optionally substituted —(C₁-C₂alkylene)-(heteroaryl);
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring;
n is 0-4; and
p is 1 or 2.

In another embodiment is a compound of Formula (IIIc) wherein each R₁ is independently selected from the group consisting of hydrogen, halogen, optionally substituted C₁-C₆alkyl, —CF₃, —OR₁₀, —N(R₁₁)R₁₂, —C(O)R₄, —C(O)OR₁₀, and —C(O)N(R₁₁)R₁₂. In another embodiment is a compound of Formula (IIIc) wherein each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (IIIc) wherein each R₁ is independently selected from the group consisting of hydrogen, halogen, and —CF₃. In another embodiment is a compound of Formula (IIIc) wherein each R₁ is independently selected from the group consisting of hydrogen and halogen. In another embodiment is a compound of Formula (IIIc) wherein each R₁ is independently selected from the group consisting of hydrogen and —CF₃.

In another embodiment is a compound of Formula (IIIc) wherein n is 3 and each R₂ is independently selected from the group consisting of halogen, optionally substituted C₁-C₆alkyl, —OR₂₀, and —N(R₂₁)R₂₂. In another embodiment is a compound of Formula (IIIc) wherein n is 3 and each R₂ is independently selected from the group consisting of halogen and optionally substituted C₁-C₆alky. In another embodiment is a compound of Formula (IIIc) wherein n is 2 and each R₂ is independently selected from the group consisting of halogen, optionally substituted C₁-C₆alkyl, —OR₂₀, and —N(R₂₁)R₂₂. In another embodiment is a compound of Formula (IIIc) wherein n is 2 and each R₂ is independently selected from the group consisting of halogen and optionally substituted C₁-C₆alkyl. In another embodiment is a compound of Formula (IIIc) wherein n is 1 and R₂ is selected from the group consisting of halogen, optionally substituted C₁-C₆alkyl, —OR₂₀, and —N(R₂₁)R₂₂. In another embodiment is a compound of Formula (IIIc) wherein n is 1 and R₂ is selected from the group consisting of halogen and optionally substituted C₁-C₆alkyl. In another embodiment is a compound of Formula (IIIc) wherein n is 0.

In another embodiment is a compound of Formula (IIIc) wherein R₃ is selected from the group consisting of hydrogen and optionally substituted C₁-C₆alkyl. In another embodiment is a compound of Formula (IIIc) wherein R₃ is hydrogen. In another embodiment is a compound of Formula (IIIc) wherein R₃ is optionally substituted C₁-C₆alkyl. In another embodiment is a compound of Formula (IIIc) wherein R₃ is methyl.

In another embodiment is a compound of Formula (IIIc) wherein

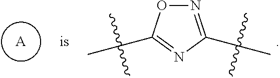

In another embodiment is a compound of Formula (IIIc) wherein

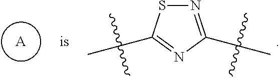

In another embodiment is a compound of Formula (IIIc) wherein

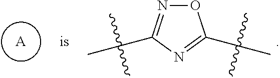

In another embodiment is a compound of Formula (IIIc) wherein

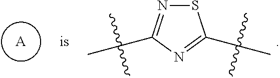

In another embodiment is a compound of Formula (IIIc) wherein

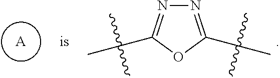

In another embodiment is a compound of Formula (IIIc) wherein

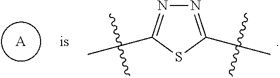

In another embodiment is a compound of Formula (IIIc) wherein p is 1. In another embodiment is a compound of Formula (IIIc) wherein p is 2.

In some embodiments, provided herein is a compound of Formula (IIId), or a pharmaceutically acceptable salt or solvate thereof:

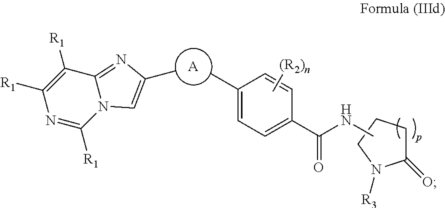

wherein:

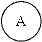

is selected from

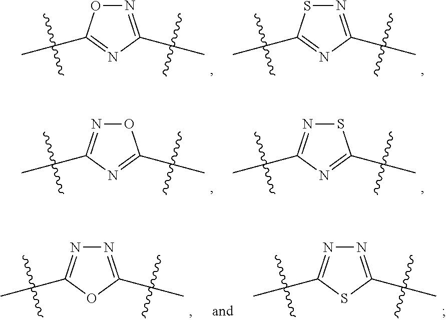 [SMILES: *c1noc(*)n1 ; *c1nsc(*)n1 ; *c1noc(*)n1 ; *c1nsc(*)n1 ; *c1nnc(*)o1 ; *c1nnc(*)s1]

each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₁₂, —N(R₁₃)N(R₁₁)S(O)₂R₁₅, —C(O)R₁₄, —C(O)OR₁₀, —C(S)OR₁₀, —C(O)SR₁₀, —C(O)N(R₁₁)R₁₂, —C(S)N(R₁₁)R₁₂, —C(O)N(R₁)S(O)₂R₁₅, —C(S)N(R₁)S(O)₂R₁₅, —C(O)N(R₁₃)N(R₁₁)R₁₂, —C(S)N(R₁₃)N(R₁₁)R₁₂, and —C(O)N(R₁₃)N(R₁)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, and optionally substituted —(C₁-C₂alkylene)-(heteroaryl);
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring;
n is 0-4; and
p is 1 or 2.

In another embodiment is a compound of Formula (IIId) wherein each R₁ is independently selected from the group consisting of hydrogen, halogen, optionally substituted C₁-C₆alkyl, —CF₃, —OR₁₀, —N(R₁₁)R₁₂, —C(O)R₁₄, —C(O)OR₁₀, and —C(O)N(R₁₁)R₁₂. In another embodiment is a compound of Formula (IIId) wherein each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (IIId) wherein each R₁ is independently selected from the group consisting of hydrogen, halogen, and —CF₃. In another embodiment is a compound of Formula (IIId) wherein each R₁ is independently selected from the group consisting of hydrogen and halogen. In another embodiment is a compound of Formula (IIId) wherein each R₁ is independently selected from the group consisting of hydrogen and —CF₃.

In another embodiment is a compound of Formula (IIId) wherein n is 3 and each R₂ is independently selected from the group consisting of halogen, optionally substituted C₁-C₆alkyl, —OR₂₀, and —N(R₂₁)R₂₂. In another embodiment is a compound of Formula (IIId) wherein n is 3 and each R₂ is independently selected from the group consisting of halogen and optionally substituted C₁-C₆alky. In another embodiment is a compound of Formula (IIId) wherein n is 2 and each R₂ is independently selected from the group consisting of halogen, optionally substituted C₁-C₆alkyl, —OR₂₀, and —N(R₂₁)R₂₂. In another embodiment is a compound of Formula (IIId) wherein n is 2 and each R₂ is independently selected from the group consisting of halogen and optionally substituted C₁-C₆alkyl. In another embodiment is a compound of Formula (IIId) wherein n is 1 and R₂ is selected from the group consisting of halogen, optionally substituted C₁-C₆alkyl, —OR₂₀, and —N(R₂₁)R₂₂. In another embodiment is a compound of Formula (IIId) wherein n is 1 and R₂ is selected from the group consisting of halogen and optionally substituted C₁-C₆alkyl. In another embodiment is a compound of Formula (IIId) wherein n is 0.

In another embodiment is a compound of Formula (IIId) wherein R₃ is selected from the group consisting of hydrogen and optionally substituted C₁-C₆alkyl. In another embodiment is a compound of Formula (IIId) wherein R₃ is hydrogen. In another embodiment is a compound of Formula (IIId) wherein R₃ is optionally substituted C₁-C₆alkyl. In another embodiment is a compound of Formula (IIId) wherein R₃ is methyl.

In another embodiment is a compound of Formula (IIId) wherein

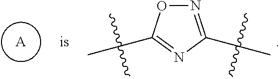

In another embodiment is a compound of Formula (IIId) wherein

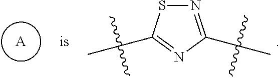

In another embodiment is a compound of Formula (IIId) wherein

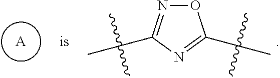

In another embodiment is a compound of Formula (IIId) wherein

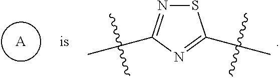

In another embodiment is a compound of Formula (IIId) wherein

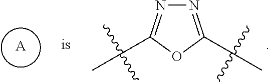

In another embodiment is a compound of Formula (IIId) wherein

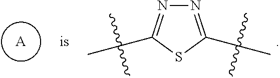

In another embodiment is a compound of Formula (IIId) wherein p is 1. In another embodiment is a compound of Formula (IIId) wherein p is 2.

In some embodiments, provided herein is a compound of Formula (IIIe), or a pharmaceutically acceptable salt or solvate thereof:

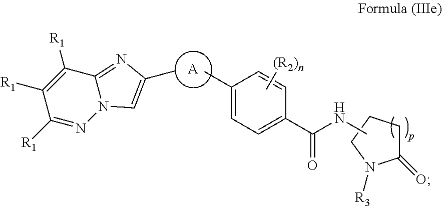

wherein:

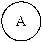

is selected from

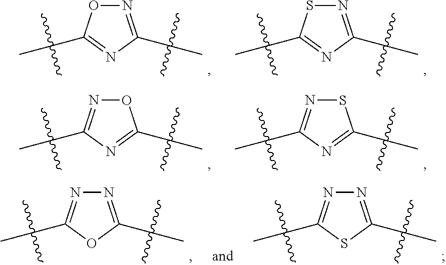

each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₁₂, —N(R₁₃)N(R₁₁)S(O)₂R₁₅, —C(O)R₄, —C(O)OR₁₀, —C(S)OR₁₀, —C(O)SR₁₀, —C(O)N(R₁₁)R₁₂, —C(S)N(R₁₁)R₁₂, —C(O)N(R₁₁)S(O)₂R₁₅, —C(S)N(R₁₁)S(O)₂R₁₅, —C(O)N(R₁₃)N(R₁₁)Ru, —C(S)N(R₁₃)N(R₁₁)R₁₂, and —C(O)N(R₁₃)N(R₁₁)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, and optionally substituted —(C₁-C₂alkylene)-(heteroaryl);
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring;
n is 0-4; and
p is 1 or 2.

In another embodiment is a compound of Formula (IIIe) wherein each R₁ is independently selected from the group consisting of hydrogen, halogen, optionally substituted C₁-C₆alkyl, —CF₃, —OR₁₀, —N(R₁₁)R₁₂, —C(O)R₁₄, —C(O)OR₁₀, and —C(O)N(R₁₁)R₁₂. In another embodiment is a compound of Formula (IIIe) wherein each R₁ is independently selected from the group consisting of hydrogen, halogen, optionally substituted C₁-C₆alkyl, —CF₃, —OR₁₀, and —N(R₁₁)R₁₂. In another embodiment is a compound of Formula (IIIe) wherein each R₁ is independently selected from the group consisting of hydrogen, halogen, and —CF₃. In another embodiment is a compound of Formula (IIIe) wherein each R₁ is independently selected from the group consisting of hydrogen and halogen. In another embodiment is a compound of Formula (IIIe) wherein each R₁ is independently selected from the group consisting of hydrogen and —CF₃.

In another embodiment is a compound of Formula (IIIe) wherein n is 3 and each R₂ is independently selected from the group consisting of halogen, optionally substituted C₁-C₆alkyl, —OR₂₀, and —N(R₂₁)R₂₂. In another embodiment is a compound of Formula (IIIe) wherein n is 3 and each R₂ is independently selected from the group consisting of halogen and optionally substituted C₁-C₆alkyl. In another embodiment is a compound of Formula (IIIe) wherein n is 2 and each R₂ is independently selected from the group consisting of halogen, optionally substituted C₁-C₆alkyl, —OR₂₀, and —N(R₂₁)R₂₂. In another embodiment is a compound of Formula (IIIe) wherein n is 2 and each R₂ is independently selected from the group consisting of halogen and optionally substituted C₁-C₆alkyl. In another embodiment is a compound of Formula (IIIe) wherein n is 1 and R₂ is selected from the group consisting of halogen, optionally substituted C₁-C₆alkyl, —OR₂₀, and —N(R₂₁)R₂₂. In another embodiment is a compound of Formula (IIIe) wherein n is 1 and R₂ is selected from the group consisting of halogen and optionally substituted C₁-C₆alkyl. In another embodiment is a compound of Formula (IIIe) wherein n is 0.

In another embodiment is a compound of Formula (IIIe) wherein R₃ is selected from the group consisting of hydrogen and optionally substituted C₁-C₆alkyl. In another embodiment is a compound of Formula (IIIe) wherein R₃ is hydrogen. In another embodiment is a compound of Formula (IIIe) wherein R₃ is optionally substituted C₁-C₆alkyl. In another embodiment is a compound of Formula (IIIe) wherein R₃ is methyl.

In another embodiment is a compound of Formula (IIIe) wherein

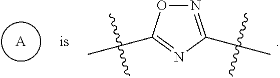

In another embodiment is a compound of Formula (IIIe) wherein

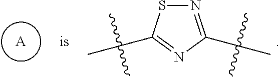

In another embodiment is a compound of Formula (IIIe) wherein

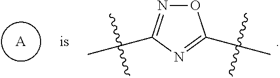

In another embodiment is a compound of Formula (IIIe) wherein

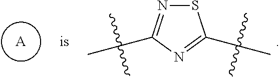

In another embodiment is a compound of Formula (IIIe) wherein

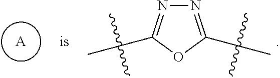

In another embodiment is a compound of Formula (IIIe) wherein

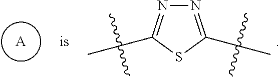

In another embodiment is a compound of Formula (IIIe) wherein p is 1. In another embodiment is a compound of Formula (IIIe) wherein p is 2.

Any combination of the groups described above for the various variables is contemplated herein. Throughout the specification, groups and substituents thereof can be chosen by one skilled in the field to provide stable moieties and compounds.

In some embodiments is a compound selected from:

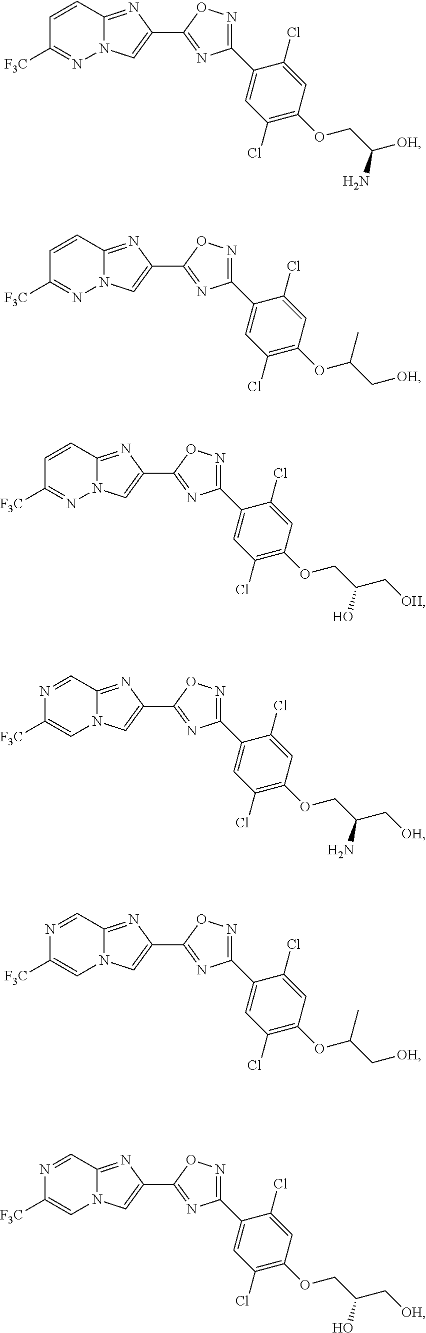 [SMILES: N[C@H](O)COc1cc(Cl)c(-c2noc(-c3cn4nc(C(F)(F)F)ccc4n3)n2)cc1Cl ; CC(CO)Oc1cc(Cl)c(-c2noc(-c3cn4nc(C(F)(F)F)ccc4n3)n2)cc1Cl ; OC[C@H](O)COc1cc(Cl)c(-c2noc(-c3cn4nc(C(F)(F)F)ccc4n3)n2)cc1Cl ; N[C@H](CO)COc1cc(Cl)c(-c2noc(-c3cn4cc(C(F)(F)F)ncc4n3)n2)cc1Cl ; CC(CO)Oc1cc(Cl)c(-c2noc(-c3cn4cc(C(F)(F)F)ncc4n3)n2)cc1Cl ; OC[C@H](O)COc1cc(Cl)c(-c2noc(-c3cn4cc(C(F)(F)F)ncc4n3)n2)cc1Cl]

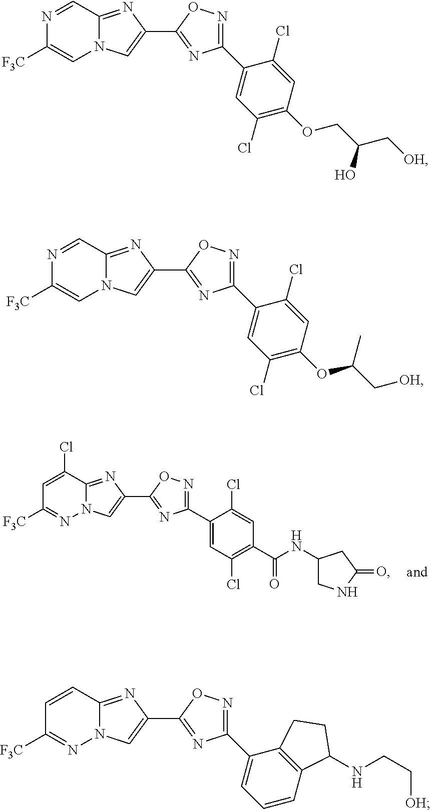 [SMILES: OC[C@@H](O)COc1cc(Cl)c(-c2noc(-c3cn4cc(C(F)(F)F)ncc4n3)n2)cc1Cl ; C[C@@H](CO)Oc1cc(Cl)c(-c2noc(-c3cn4cc(C(F)(F)F)ncc4n3)n2)cc1Cl ; O=C1CC(NC(=O)c2cc(Cl)c(-c3noc(-c4cn5nc(C(F)(F)F)cc(Cl)c5n4)n3)cc2Cl)CN1 ; "and" ; OCCNC1CCc2c(-c3noc(-c4cn5nc(C(F)(F)F)ccc5n4)n3)cccc21]

a pharmaceutically acceptable salt, or pharmaceutically acceptable solvate thereof.

In some embodiments is a compound selected from:

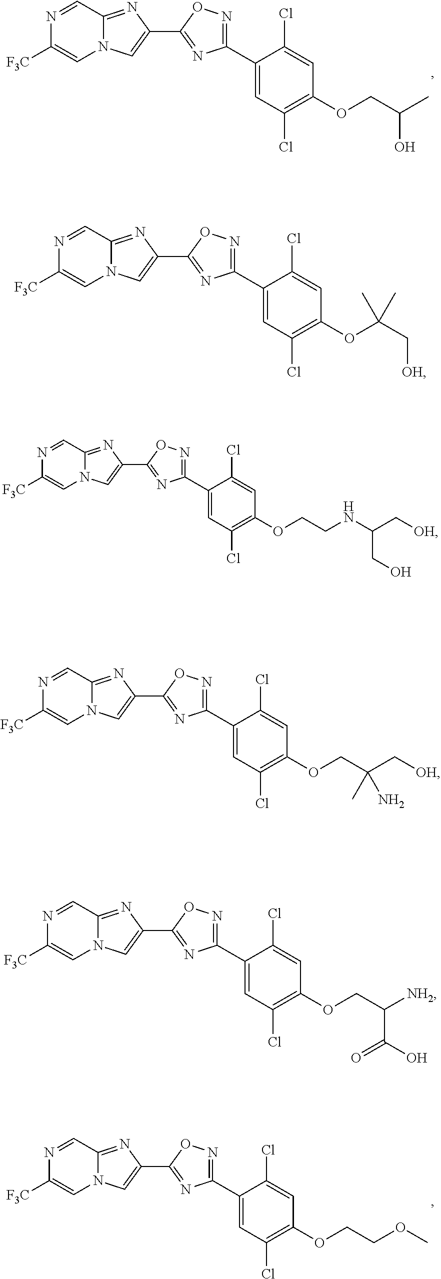 [SMILES: CC(O)COc1cc(Cl)c(-c2noc(-c3cn4cc(C(F)(F)F)ncc4n3)n2)cc1Cl ; CC(C)(CO)Oc1cc(Cl)c(-c2noc(-c3cn4cc(C(F)(F)F)ncc4n3)n2)cc1Cl ; OCC(CO)NCCOc1cc(Cl)c(-c2noc(-c3cn4cc(C(F)(F)F)ncc4n3)n2)cc1Cl ; CC(N)(CO)COc1cc(Cl)c(-c2noc(-c3cn4cc(C(F)(F)F)ncc4n3)n2)cc1Cl ; NC(COc1cc(Cl)c(-c2noc(-c3cn4cc(C(F)(F)F)ncc4n3)n2)cc1Cl)C(=O)O ; COCCOc1cc(Cl)c(-c2noc(-c3cn4cc(C(F)(F)F)ncc4n3)n2)cc1Cl]

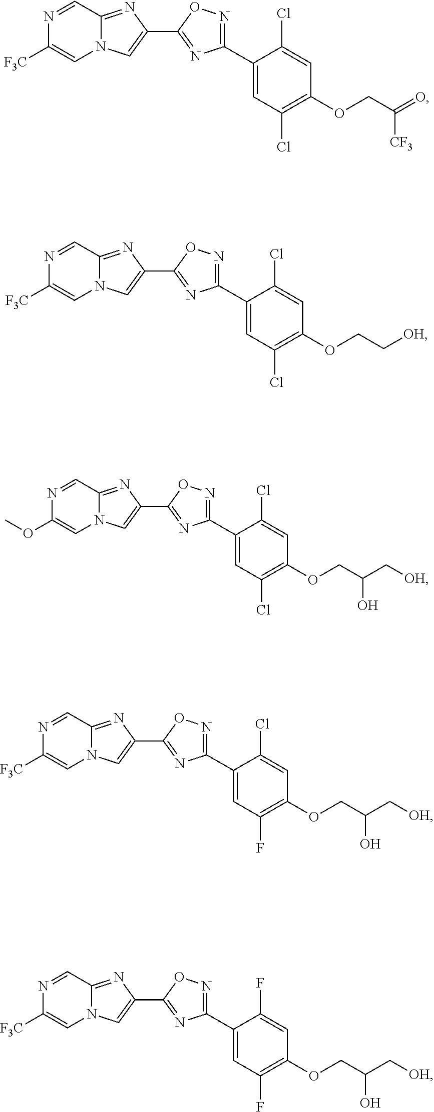 [SMILES: O=C(COc1cc(Cl)c(-c2noc(-c3cn4cc(C(F)(F)F)ncc4n3)n2)cc1Cl)C(F)(F)F ; OCCOc1cc(Cl)c(-c2noc(-c3cn4cc(C(F)(F)F)ncc4n3)n2)cc1Cl ; COc1cn2cc(-c3nc(-c4cc(Cl)c(OCC(O)CO)cc4Cl)no3)nc2cn1 ; OCC(O)COc1cc(Cl)c(-c2noc(-c3cn4cc(C(F)(F)F)ncc4n3)n2)cc1F ; OCC(O)COc1cc(F)c(-c2noc(-c3cn4cc(C(F)(F)F)ncc4n3)n2)cc1F]

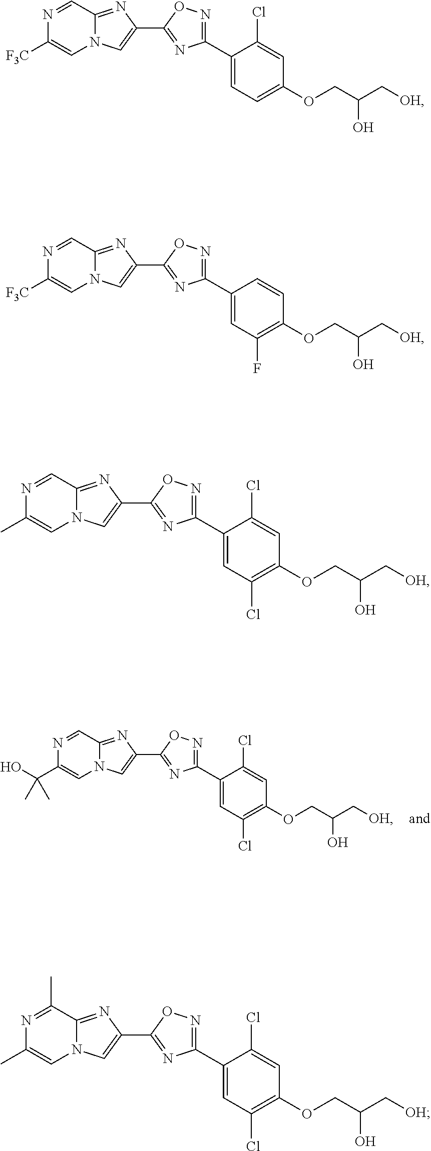 [SMILES: OCC(O)COc1ccc(-c2noc(-c3cn4cc(C(F)(F)F)ncc4n3)n2)c(Cl)c1 ; OCC(O)COc1ccc(-c2noc(-c3cn4cc(C(F)(F)F)ncc4n3)n2)cc1F ; Cc1cn2cc(-c3nc(-c4cc(Cl)c(OCC(O)CO)cc4Cl)no3)nc2cn1 ; CC(C)(O)c1cn2cc(-c3nc(-c4cc(Cl)c(OCC(O)CO)cc4Cl)no3)nc2cn1 ; "and" ; Cc1cn2cc(-c3nc(-c4cc(Cl)c(OCC(O)CO)cc4Cl)no3)nc2c(C)n1]

or a pharmaceutically acceptable salt, or pharmaceutically acceptable solvate thereof.

In some embodiments, the therapeutic agent(s) (e.g. compound of Formula (I), (Ia), (Ib), (Ic), (Id), (II), (IIa), (IIb), (IIc), (IId), (III), (IIIa), (IIIb), (IIIc), (IIId), or (IIIe)) is present in the pharmaceutical composition as a pharmaceutically acceptable salt. In some embodiments, any compound described above is suitable for any method or composition described herein.

In certain embodiments, the compounds presented herein possess one or more stereocenters and each center independently exists in either the R or S configuration. The compounds presented herein include all diastereomeric, enantiomeric, and epimeric forms as well as the appropriate mixtures thereof. Stereoisomers are obtained, if desired, by methods such as, stereoselective synthesis and/or the separation of stereoisomers by chiral chromatographic columns. In some embodiments, a compound of Formula (I), (Ia), (Ib), (Ic), (Id), (II), (IIa), (IIb), (IIc), (IId), (III), (IIIa), (IIIb), (IIIc), (IIId), or (IIIe) is used as a single enantiomer. In some embodiments, a compound of Formula (I), (Ia), (Ib), (Ic), (Id), (II), (Ia), (IIb), (IIc), (IId), (III), (IIIa), (IIIb), (IIIc), (IIId), or (IIIe) is used as a racemic mixture.

The methods and formulations described herein include the use of N-oxides (if appropriate), crystalline forms (also known as polymorphs), or pharmaceutically acceptable salts of compounds having the structures presented herein, as well as active metabolites of these compounds having the same type of activity.

In some situations, compounds may exist as tautomers. All tautomers are included within the scope of the compounds presented herein.

In some embodiments, compounds described herein are prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. In some embodiments, the design of a prodrug increases the effective water solubility. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs of the compounds described herein include, but are not limited to, esters, ethers, carbonates, thiocarbonates, N-acyl derivatives, N-acyloxyalkyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like.

Prodrug forms of the herein described compounds, wherein the prodrug is metabolized in vivo to produce a compound of Formula (I), (Ia), (Ib), (Ic), (Id), (II), (IIa), (IIb), (IIc), (IId), (III), (IIIa), (IIIb), (IIIc), (IIId), or (IIIe), as set forth herein are included within the scope of the claims. In some cases, some of the herein-described compounds may be a prodrug for another derivative or active compound.

In specific embodiments, the compounds described herein exist in solvated forms with pharmaceutically acceptable solvents such as water, ethanol, and the like. In other embodiments, the compounds described herein exist in unsolvated form.

In some embodiments, the compounds of Formula (I), (Ia), (Ib), (Ic), (Id), (II), (IIa), (IIb), (IIc), (IId), (III), (IIIa), (IIIb), (IIIc), (IIId), or (IIIe) described herein include solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol.

In some embodiments, sites on the compounds of Formula (I), (Ia), (Ib), (Ic), (Id), (II), (IIa), (IIb), (IIc), (IId), (III), (IIIa), (IIIb), (IIIc), (IIId), or (IIIe) disclosed herein are susceptible to various metabolic reactions. Therefore incorporation of appropriate substituents at the places of metabolic reactions will reduce, minimize or eliminate the metabolic pathways. In specific embodiments, the appropriate substituent to decrease or eliminate the susceptibility of the aromatic ring to metabolic reactions is, by way of example only, a halogen, deuterium or an alkyl group.

In some embodiments, the compounds of Formula (I), (Ia), (Ib), (Ic), (Id), (II), (Ia), (IIb), (IIc), (IId), (III), (IIIa), (IIIb), (IIIc), (IIId), or (IIIe) disclosed herein are isotopically-labeled, which are identical to those recited in the various formulae and structures presented herein, but for the fact that one or more atoms are replaced by an atom having an atomic mass or mass number different from the atomic mass or mass number usually found in nature. In some embodiments, one or more hydrogen atoms are replaced with deuterium. In some embodiments, metabolic sites on the compounds described herein are deuterated. In some embodiments, substitution with deuterium affords certain therapeutic advantages resulting from greater metabolic stability, such as, for example, increased in vivo half-life or reduced dosage requirements.

In some embodiments, compounds described herein, such as compounds of Formula (I), (Ia), (Ib), (Ic), (Id), (II), (IIa), (IIb), (IIc), (IId), (III), (IIIa), (IIIb), (IIIc), (IIId), or (IIIe), are in various forms, including but not limited to, amorphous forms, milled forms and nano-particulate forms. In addition, compounds described herein include crystalline forms, also known as polymorphs. Polymorphs include the different crystal packing arrangements of the same elemental composition of a compound. Polymorphs usually have different X-ray diffraction patterns, melting points, density, hardness, crystal shape, optical properties, stability, and solubility. Various factors such as the recrystallization solvent, rate of crystallization, and storage temperature may cause a single crystal form to dominate.

The screening and characterization of the pharmaceutically acceptable salts, polymorphs and/or solvates may be accomplished using a variety of techniques including, but not limited to, thermal analysis, x-ray diffraction, spectroscopy, vapor sorption, and microscopy. Thermal analysis methods address thermo chemical degradation or thermo physical processes including, but not limited to, polymorphic transitions, and such methods are used to analyze the relationships between polymorphic forms, determine weight loss, to find the glass transition temperature, or for excipient compatibility studies. Such methods include, but are not limited to, Differential scanning calorimetry (DSC), Modulated Differential Scanning Calorimetry (MDCS), Thermogravimetric analysis (TGA), and Thermogravi-metric and Infrared analysis (TG/IR). X-ray diffraction methods include, but are not limited to, single crystal and powder diffractometers and synchrotron sources. The various spectroscopic techniques used include, but are not limited to, Raman, FTR, UV-VIS, and NMR (liquid and solid state). The various microscopy techniques include, but are not limited to, polarized light microscopy, Scanning Electron Microscopy (SEM) with Energy Dispersive X-Ray Analysis (EDX), Environmental Scanning Electron Microscopy with EDX (in gas or water vapor atmosphere), IR microscopy, and Raman microscopy.

Throughout the specification, groups and substituents thereof can be chosen to provide stable moieties and compounds.

### Synthesis of Compounds

In some embodiments, the synthesis of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, FischerScientific (Fischer Chemicals), and AcrosOrganics. In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compound as disclosed herein may be derived from reactions and the reactions may be modified by the use of appropriate reagents and conditions, for the introduction of the various moieties found in the formulae as provided herein.

### Use of Protecting Groups

In the reactions described, it may be necessary to protect reactive functional groups, for example hydroxy, amino, imino, thio or carboxy groups, where these are desired in the final product, in order to avoid their unwanted participation in reactions. Protecting groups are used to block some or all of the reactive moieties and prevent such groups from participating in chemical reactions until the protective group is removed. It is preferred that each protective group be removable by a different means. Protective groups that are cleaved under totally disparate reaction conditions fulfill the requirement of differential removal.

Protective groups can be removed by acid, base, reducing conditions (such as, for example, hydrogenolysis), and/or oxidative conditions. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, but not limited to, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be protected by conversion to simple ester compounds as exemplified herein, which include conversion to alkyl esters, or they may be blocked with oxidatively-removable protective groups such as 2,4-dimethoxybenzyl, while co-existing amino groups may be blocked with fluoride labile silyl carbamates.

Allyl blocking groups are useful in then presence of acid- and base-protecting groups since the former are stable and can be subsequently removed by metal or pi-acid catalysts. For example, an allyl-blocked carboxylic acid can be deprotected with a Pd⁰-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typically blocking/protecting groups may be selected from:

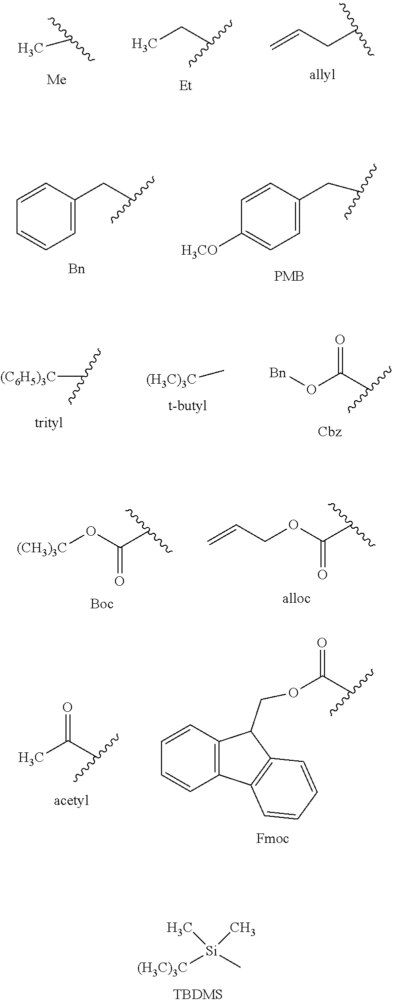

Other protecting groups, plus a detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure).

### Certain Terminology

Unless defined otherwise, all technical and scientific terms used herein have the same meaning as is commonly understood to which the claimed subject matter belongs. In the event that there are a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described.

Definition of standard chemistry terms may be found in reference works, including but not limited to, Carey and Sundberg “Advanced Organic Chemistry 4^{th} Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology.

Unless specific definitions are provided, the nomenclature employed in connection with, and the laboratory procedures and techniques of, analytical chemistry, synthetic organic chemistry, and medicinal and pharmaceutical chemistry described herein are those recognized in the field. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods, compounds, compositions described herein.

As used herein, C₁-Cₓ includes C₁-C₂, C₁-C₃ . . . C₁-Cₓ. C₁-Cₓ refers to the number of carbon atoms that make up the moiety to which it designates (excluding optional substituents).

An “alkyl” group refers to an aliphatic hydrocarbon group. The alkyl groups may or may not include units of unsaturation. The alkyl moiety may be a “saturated alkyl” group, which means that it does not contain any units of unsaturation (i.e. a carbon-carbon double bond or a carbon-carbon triple bond). The alkyl group may also be an “unsaturated alkyl” moiety, which means that it contains at least one unit of unsaturation. The alkyl moiety, whether saturated or unsaturated, may be branched, straight chain, or cyclic.

The “alkyl” group may have 1 to 6 carbon atoms (whenever it appears herein, a numerical range such as “1 to 6” refers to each integer in the given range; e.g., “1 to 6 carbon atoms” means that the alkyl group may consist of 1 carbon atom, 2 carbon atoms, 3 carbon atoms, etc., up to and including 6 carbon atoms, although the present definition also covers the occurrence of the term “alkyl” where no numerical range is designated). The alkyl group of the compounds described herein may be designated as “C₁-C₆ alkyl” or similar designations. By way of example only, “C₁-C₆ alkyl” indicates that there are one to six carbon atoms in the alkyl chain, i.e., the alkyl chain is selected from the group consisting of methyl, ethyl, n-propyl, iso-propyl, n-butyl, iso-butyl, sec-butyl, t-butyl, n-pentyl, iso-pentyl, neo-pentyl, hexyl, propen-3-yl (allyl), cyclopropylmethyl, cyclobutylmethyl, cyclopentylmethyl, cyclohexylmethyl. Alkyl groups can be substituted or unsubstituted. Depending on the structure, an alkyl group can be a monoradical or a diradical (i.e., an alkylene group).

An “alkoxy” refers to a “—O-alkyl” group, where alkyl is as defined herein.

The term “alkenyl” refers to a type of alkyl group in which two atoms of the alkyl group form a double bond that is not part of an aromatic group. Non-limiting examples of an alkenyl group include —CH═CH₂, —C(CH₃)═CH₂, —CH═CHCH₃, —CH═C(CH₃)₂ and —C(CH₃)═CHCH₃. The alkenyl moiety may be branched, straight chain, or cyclic (in which case, it would also be known as a “cycloalkenyl” group). Alkenyl groups may have 2 to 6 carbons. Alkenyl groups can be substituted or unsubstituted. Depending on the structure, an alkenyl group can be a monoradical or a diradical (i.e., an alkenylene group).

The term “alkynyl” refers to a type of alkyl group in which the two atoms of the alkyl group form a triple bond. Non-limiting examples of an alkynyl group include —C≡CH, —C≡CCH₃, —C≡CCH₂CH₃ and —C≡CCH₂CH₂CH₃. The “R” portion of the alkynyl moiety may be branched, straight chain, or cyclic. An alkynyl group can have 2 to 6 carbons. Alkynyl groups can be substituted or unsubstituted. Depending on the structure, an alkynyl group can be a monoradical or a diradical (i.e., an alkynylene group).

“Amino” refers to a —NH₂ group.

The term “alkylamine” or “alkylamino” refers to the —N(alkyl)ₓH_{y} group, where alkyl is as defined herein and x and y are selected from the group x=1, y=1 and x=2, y=0. When x=2, the alkyl groups, taken together with the nitrogen to which they are attached, can optionally form a cyclic ring system. “Dialkylamino” refers to a —N(alkyl)₂ group, where alkyl is as defined herein.

The term “aromatic” refers to a planar ring having a delocalized 7-electron system containing 4n+2π electrons, where n is an integer. Aromatic rings can be formed from five, six, seven, eight, nine, or more than nine atoms. Aromatics can be optionally substituted. The term “aromatic” includes both aryl groups (e.g., phenyl, naphthalenyl) and heteroaryl groups (e.g., pyridinyl, quinolinyl).

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, and naphthalenyl. Depending on the structure, an aryl group can be a monoradical or a diradical (i.e., an arylene group).

“Carboxy” refers to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to,

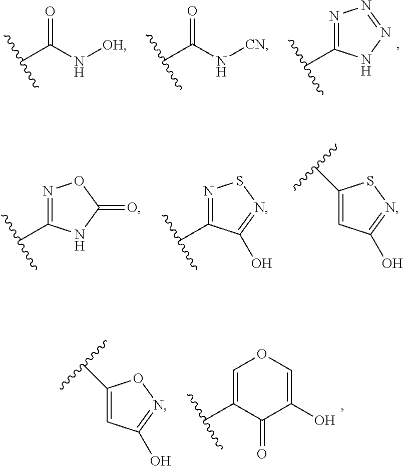

and the like.

The term “cycloalkyl” refers to a monocyclic or polycyclic non-aromatic radical, wherein each of the atoms forming the ring (i.e. skeletal atoms) is a carbon atom. Cycloalkyls may be saturated, or partially unsaturated. Cycloalkyls may be fused with an aromatic ring (in which case the cycloalkyl is bonded through a non-aromatic ring carbon atom). Cycloalkyl groups include groups having from 3 to 10 ring atoms. Illustrative examples of cycloalkyl groups include, but are not limited to, the following moieties:

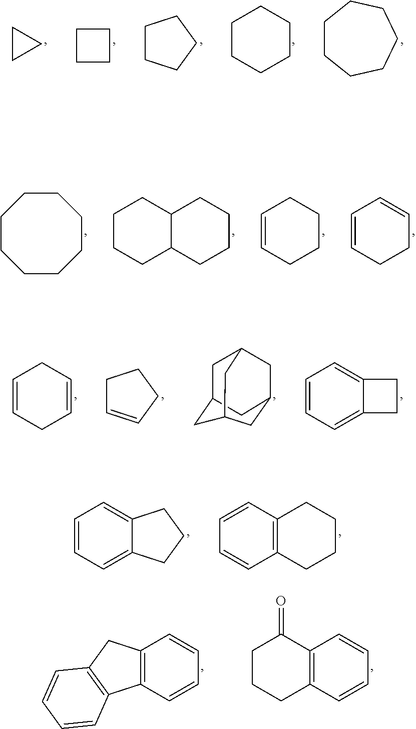

and the like.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. Polycyclic heteroaryl groups may be fused or non-fused. Illustrative examples of heteroaryl groups include the following moieties:

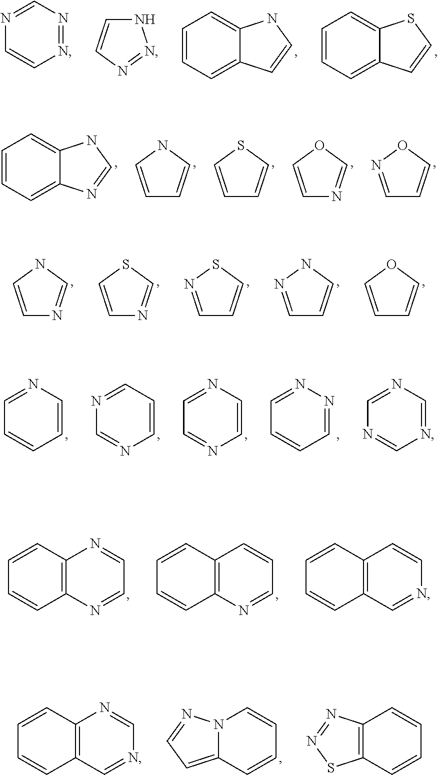

and the like.

A “heterocycloalkyl” group or “heteroalicyclic” group refers to a cycloalkyl group, wherein at least one skeletal ring atom is a heteroatom selected from nitrogen, oxygen and sulfur. The radicals may be fused with an aryl or heteroaryl. Illustrative examples of heterocycloalkyl groups, also referred to as non-aromatic heterocycles, include:

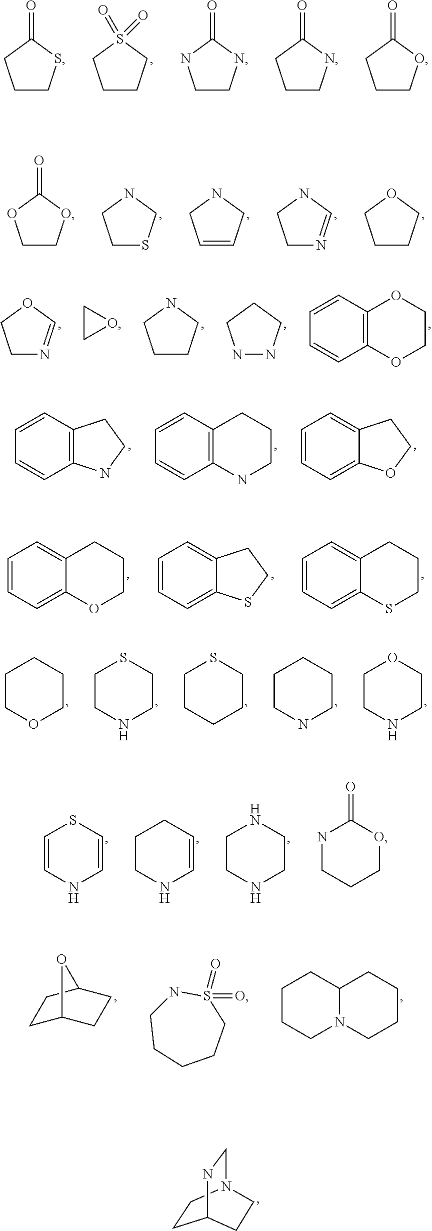

and the like. The term heteroalicyclic also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring).

The term “halo” or, alternatively, “halogen” means fluoro, chloro, bromo and iodo.

The term “haloalkyl” refers to an alkyl group that is substituted with one or more halogens. The halogens may the same or they may be different. Non-limiting examples of haloalkyls include —CH₂Cl, —CF₃, —CHF₂, —CH₂CF₃, —CF₂CF₃, —CF(CH₃)₂, and the like.

The terms “fluoroalkyl” and “fluoroalkoxy” include alkyl and alkoxy groups, respectively, that are substituted with one or more fluorine atoms. Non-limiting examples of fluoroalkyls include —CF₃, —CHF₂, —CH₂F, —CH₂CF₃, —CF₂CF₃, —CF₂CF₂CF₃, —CF(CH₃)₃, and the like. Non-limiting examples of fluoroalkoxy groups, include —OCF₃, —OCHF₂, —OCH₂F, —OCH₂CF₃, —OCF₂CF₃, —OCF₂CF₂CF₃, —OCF(CH₃)₂, and the like.

The term “heteroalkyl” refers to an alkyl radical where one or more skeletal chain atoms is selected from an atom other than carbon, e.g., oxygen, nitrogen, sulfur, phosphorus, silicon, or combinations thereof. The heteroatom(s) may be placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH₂—NH—OCH₃, —CH₂—O—Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃. Excluding the number of heteroatoms, a “heteroalkyl” may have from 1 to 6 carbon atoms.

The term “bond” or “single bond” refers to a chemical bond between two atoms, or two moieties when the atoms joined by the bond are considered to be part of larger substructure.

The term “moiety” refers to a specific segment or functional group of a molecule. Chemical moieties are often recognized chemical entities embedded in or appended to a molecule.

As used herein, the substituent “R” appearing by itself and without a number designation refers to a substituent selected from among from alkyl, haloalkyl, heteroalkyl, alkenyl, cycloalkyl, aryl, heteroaryl (bonded through a ring carbon), and heterocycloalkyl.

The term “optionally substituted” or “substituted” means that the referenced group may be substituted with one or more additional group(s) individually and independently selected from alkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆alkylalkyne, halo, acyl, acyloxy, —CO₂H, —CO₂-alkyl, nitro, haloalkyl, fluoroalkyl, and amino, including mono- and di-substituted amino groups (e.g. —NH₂, —NHR, —N(R)₂), and the protected derivatives thereof. In some embodiments, optional substituents are independently selected from halogen, —CN, —NH₂, —NH(CH₃), —N(CH₃)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some embodiments, optional substituents are independently selected from halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic, saturated or unsaturated carbon atoms, excluding aromatic carbon atoms) includes oxo (═O).

The methods and formulations described herein include the use of crystalline forms (also known as polymorphs), or pharmaceutically acceptable salts of compounds having the structure of Formula (I), (Ia), (Ib), (Ic), (Id), (II), (IIa), (IIb), (IIc), (IId), (III), (IIIa), (IIIb), (IIIc), (IIId), or (IIIe), as well as active metabolites of these compounds having the same type of activity.

As used herein, the term “about” or “approximately” means within 20%, preferably within 10%, and more preferably within 5% of a given value or range.

The term a “therapeutically effective amount” as used herein refers to the amount of an S1P receptor modulator that, when administered to a mammal in need, is effective to at least partially ameliorate or to at least partially prevent diseases, disorders or conditions described herein.

As used herein, the term “expression” includes the process by which polynucleotides are transcribed into mRNA and translated into peptides, polypeptides, or proteins.

The term “activator” is used in this specification to denote any molecular species that results in activation of the indicated receptor, regardless of whether the species itself binds to the receptor or a metabolite of the species binds to the receptor. Thus, the activator can be a ligand of the receptor or it can be an activator that is metabolized to the ligand of the receptor, i.e., a metabolite that is formed in tissue and is the actual ligand.

The term “antagonist” as used herein, refers to a small-molecule agent that binds to a receptor and subsequently decreases the agonist induced transcriptional activity of the receptor.

The term “agonist” as used herein, refers to a small-molecule agent that binds to a receptor and subsequently increases receptor transcriptional activity in the absence of a known agonist.

The term “inverse agonist” as used herein, refers to a small-molecule agent that binds to a receptor and subsequently decreases the basal level of receptor transcriptional activity that is present in the absence of a known agonist.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “S1P receptor modulator” includes S1P receptor agonists, partial agonists, antagonists and tissue selective S1P receptor modulators.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. Those skilled in the art recognize that a therapy which reduces the severity of a pathology in one species of mammal is predictive of the effect of the therapy on another species of mammal.

The terms “treat,” “treating” or “treatment,” as used herein, include alleviating, abating or ameliorating at least one symptom of a disease disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

### Pharmaceutical Compositions and Methods of Administration of S1P Receptor Modulators

Administration of S1P receptor modulators as described herein can be in any pharmacological form including a therapeutically effective amount of an S1P receptor modulator alone or in combination with a pharmaceutically acceptable carrier.

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Additional details about suitable excipients for pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of a compound of Formula (I), (Ia), (Ib), (Ic), (Id), (II), (IIa), (IIb), (IIc), (IId), (III), (IIIa), (IIIb), (IIIc), (IIId), or (IIIe) described herein, with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors.

In another aspect, provided herein is a pharmaceutical composition comprising a compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, and a pharmaceutically acceptable diluent, excipient or binder. In one embodiment, the pharmaceutical composition comprising the compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, is formulated for a route of administration selected from oral administration, parenteral administration, buccal administration, nasal administration, topical administration, or rectal administration.

In another aspect is a method of treating a disease, disorder or condition in a mammal that would benefit from S1P receptor modulation comprising administering to the mammal a therapeutically effective amount of a compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof.

In another embodiment is a method of treating a disease, disorder or condition in a mammal that would benefit from S1P receptor modulation comprising administering to the mammal a therapeutically effective amount of a compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, wherein the disease, disorder or condition in a mammal is selected from multiple sclerosis, ulcerative colitis, and Crohn's disease. In another embodiment is a method of treating a disease, disorder or condition in a mammal that would benefit from S1P receptor modulation comprising administering to the mammal a therapeutically effective amount of a compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, wherein the disease, disorder or condition in a mammal is multiple sclerosis. In another embodiment is a method of treating a disease, disorder or condition in a mammal that would benefit from S1P receptor modulation comprising administering to the mammal a therapeutically effective amount of a compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, wherein the disease, disorder or condition in a mammal is ulcerative colitis. In another embodiment is a method of treating a disease, disorder or condition in a mammal that would benefit from S1P receptor modulation comprising administering to the mammal a therapeutically effective amount of a compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof; wherein the disease, disorder or condition in a mammal is Crohn's disease.

In a further embodiment is a method of treating a disease, disorder or condition in a mammal that would benefit from S1P receptor modulation comprising administering to the mammal a therapeutically effective amount of a compound of Formula (I), (Ia), (Ib), (Ic), (Id), (II), (IIa), (IIb), (IIc), (IId), (III), (IIIa), (IIIb), (IIIc), (IIId), or (IIIe), or a pharmaceutically acceptable salt or solvate thereof, wherein the disease, disorder or condition in a mammal is rejection of transplanted organs or tissue; graft-versus-host diseases brought about by transplantation; autoimmune syndromes including rheumatoid arthritis, multiple sclerosis, myasthenia gravis; pollen allergies; type I diabetes; prevention of psoriasis; Crohn's disease; ulcerative colitis, acute respiratory distress syndrome; adult respiratory distress syndrome; influenza; post-infectious autoimmune diseases including rheumatic fever and post-infectious glomerulonephritis; and metastasis of carcinoma.

In some embodiments a compound of Formula (I), (Ia), (Ib), (Ic), (Id), (II), (Ia), (IIb), (IIc), (IId), (III), (IIIa), (IIIb), (IIIc), (IIId), or (IIIe) is used singly or in combination with one or more therapeutic agents as components of mixtures (as in combination therapy). In some embodiments a compound of Formula (I), (Ia), (Ib), (Ic), (Id), (II), (IIa), (IIb), (IIc), (IId), (III), (IIIa), (IIIb), (IIIc), (IIId), or (IIIe) is used singly. In some embodiments a compound of Formula (I), (Ia), (Ib), (Ic), (Id), (II), (IIa), (IIb), (IIc), (IId), (III), (IIIa), (IIIb), (IIIc), (IIId), or (IIIe) is used in combination with another S1P receptor modulator or another type of therapeutic agent, or both. In some embodiments a compound of Formula (I), (Ia), (Ib), (Ic), (Id), (II), (IIa), (IIb), (IIc), (IId), (III), (IIIa), (IIIb), (IIIc), (IIId), or (IIIe) is used in combination with another S1P receptor modulator. In some embodiments a compound of Formula (I), (Ia), (Ib), (Ic), (Id), (II), (IIa), (IIb), (IIc), (IId), (III), (IIIa), (IIIb), (IIIc), (IIId), or (IIIe) is used in combination with another type of therapeutic agent. In some embodiments a compound of Formula (I), (Ia), (Ib), (Ic), (Id), (II), (IIa), (IIb), (IIc), (IId), (III), (IIIa), (IIIb), (IIIc), (IIId), or (IIIe) is used in combination with another S1P receptor modulator and another type of therapeutic agent.

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. Moreover, the pharmaceutical compositions described herein, which include a compound of Formula (I), (Ia), (Ib), (Ic), (Id), (II), (IIa), (IIb), (IIc), (IId), (III), (IIIa), (IIIb), (IIIc), (Id), or (IIIe) described herein, can be formulated into any suitable dosage form, including but not limited to, aqueous oral dispersions, liquids, gels, syrups, elixirs, slurries, suspensions, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

Dose administration can be repeated depending upon the pharmacokinetic parameters of the dosage formulation and the route of administration used.

It is especially advantageous to formulate compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on (a) the unique characteristics of the S1P receptor modulator and the particular therapeutic effect to be achieved and (b) the limitations inherent in the art of compounding such an active compound for the treatment of sensitivity in individuals. The specific dose can be readily calculated by one of ordinary skill in the art, e.g., according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the S1P receptor modulator activities disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

Toxicity and therapeutic efficacy of such S1P receptor modulators can be determined by standard pharmaceutical procedures in cell cultures or experimental animals, for example, for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. S1P receptor modulators that exhibit large therapeutic indices are preferred. While S1P receptor modulators that exhibit toxic side effects may be used, care should be taken to design a delivery system that targets such modulators to the site of affected tissue in order to minimize potential damage to uninfected cells and, thereby, reduce side effects.

The data obtained from the cell culture assays and animal studies can be used in formulating a range of dosage for use in humans. The dosage of such SIP receptor modulators lies preferably within a range of circulating concentrations that include the ED₅₀ with little or no toxicity. The dosage may vary within this range depending upon the dosage form employed and the route of administration utilized. For any S1P receptor modulator used in a method described herein, the therapeutically effective dose can be estimated initially from cell culture assays. A dose may be formulated in animal models to achieve a circulating plasma concentration range that includes the IC₅₀ (i.e., the concentration of the S1P receptor modulator that achieves a half-maximal inhibition of symptoms) as determined in cell culture. Such information can be used to more accurately determine useful doses in humans. Levels in plasma may be measured, for example, by high performance liquid chromatography.

### Methods of Dosing and Treatment Regimens

The compounds described herein can be used in the preparation of medicaments for the modulation of the S1P receptor, or for the treatment of diseases or conditions that would benefit, at least in part, from modulation of the S1P receptor. In addition, a method for treating any of the diseases or conditions described herein in a subject in need of such treatment, involves administration of pharmaceutical compositions containing at least one compound described herein, or a pharmaceutically acceptable salt, or pharmaceutically acceptable solvate or hydrate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from about 10% to about 100%, including, by way of example only, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but can nevertheless be determined in a manner recognized in the field according to the particular circumstances surrounding the case, including, e.g., the specific agent being administered, the route of administration, the condition being treated, and the subject or host being treated. In general, however, doses employed for adult human treatment will typically be in the range of about 0.01 mg per day to about 5000 mg per day, in some embodiments, about 1 mg per day to about 1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The daily dosages appropriate for the compounds described herein described herein are from about 0.001 mg/kg to about 30 mg/kg. In one embodiment, the daily dosages are from about 0.01 mg/kg to about 10 mg/kg. An indicated daily dosage in the larger mammal, including, but not limited to, humans, is in the range from about 0.1 mg to about 1000 mg, conveniently administered in a single dose or in divided doses, including, but not limited to, up to four times a day or in extended release form. Suitable unit dosage forms for oral administration include from about 1 to about 500 mg active ingredient. In one embodiment, the unit dosage is about 1 mg, about 5 mg, about, 10 mg, about 20 mg, about 50 mg, about 100 mg, about 200 mg, about 250 mg, about 400 mg, or about 500 mg. The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

### EXAMPLES

The following examples are offered for purposes of illustration, and are not intended to limit the scope of the claims provided herein. All literature citations in these examples and throughout this specification are incorporated herein by references for all legal purposes to be served thereby. The starting materials and reagents used for the synthesis of the compounds described herein may be synthesized or can be obtained from commercial sources, such as, but not limited to, Sigma-Aldrich, Acros Organics, Fluka, and Fischer Scientific.

### Example 1: Synthesis of 2-(2,5-dichloro-4-(5-(6-(trifluoromethyl)imidazo[1,2-a]pyrazin-2-yl)-1,2,4-oxadiazol-3-yl)phenoxy)propan-1-ol (7)

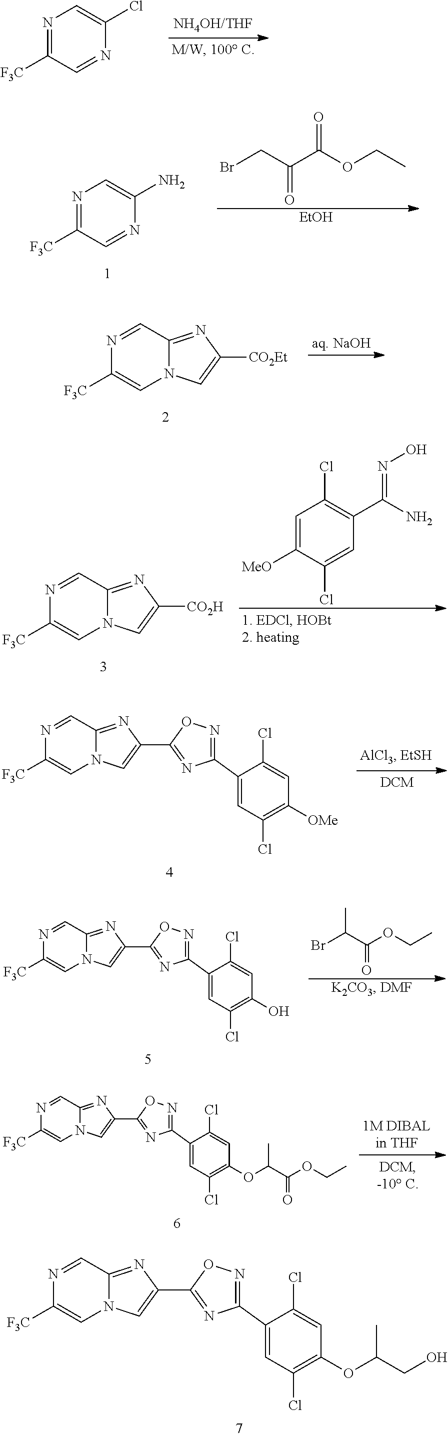

A mixture of 5-chloro-2-trifluoromethylpyrazine (2 g, 10.95 mmol) and ammonium hydroxide (12 mL) in THF (4 mL) was heated at 100° C. in a microwave reactor for 1 h. After this period, the reaction mixture was extracted with ethyl acetate and H₂O. The aqueous layer was extracted 3 times. The combined extracts were dried over with MgSO₄, filtered, and evaporated under reduce pressure to afford the amine compound 1 as a light yellow solid (1.78 g, 99%).

To a stirred solution of compound 1 (500 mg, 3.06 mmol) in EtOH (15 mL) was added ethyl bromopyruvate (0.77 mL, 6.13 mmol) at room temperature. The resulting mixture was heated to 80° C. for 16 h. The reaction mixture was cooled to ambient temperature and concentrated. The residue was suspended in diethyl ether and the resulting solid was filtered and dried under vacuum to give the ester compound 2 as a yellow solid (524 mg, 66%).

To a stirred solution of compound 2 (2 g, 7.7 mmol) in MeOH (40 mL) was added 1N NaOH (20 mL). The mixture was stirred for 1 h. After completion of the reaction, the reaction mixture was concentrated in vacuo, water was added to the residue and the mixture acidified to pH 2-3 using 1 N HCl. The resulting precipitate was filtered, washed with water, and dried to afford compound 3 as an ivory solid (1.42 g, 79%).

To a stirred solution of compound 3 (1.67 g, 7.22 mmol) in DMF (20 mL) were added EDCI (2.07 g, 10.83 mmol) and HOBt (1.46 g, 10.83 mmol). The mixture was stirred for 15 min and hydroxyimidate (2.03 g, 8.66 mmol) was added to the mixture. The reaction mixture was stirred at room temperature overnight. The reaction mixture was then stirred at 100° C. for 5 h. The reaction mixture was cooled to ambient temperature and poured into water. The resulting solid was filtered, washed with water followed by ether to afford light orange solid 4 (2.4 g, 77%).

To a cold solution of compound 4 (2.4 g, 5.58 mmol, 1.0 eq) in DCM (250 mL) was added AlCl₃ (3.7 g, 27.89 mmol, 5.0 eq) in small portions under Ar maintaining the temperature below 10° C. The light brown suspension was stirred for 10 min and then EtSH (2.06 mL, 27.89 mmol, 5.0 eq) was added dropwise maintaining the temperature below 5° C. The reaction mixture was stirred for 3 h. The reaction was quenched with ice water and extracted with ethyl acetate (×3) and H₂O. The combined organic layers were dried over MgSO₄, filtered (short silica pad) and concentrated in vacuo. The solid was suspended in ether and then filtered and dried to afford compound 5 as an ivory solid (1.71 g, 73%)

To a stirred solution of the compound 5 (200 mg, 0.48 mmol) in DMF (4 mL) were added K₂CO₃ (265 mg, 1.92 mmol) and ethyl 2-bromopropionate (0.25 mL, 1.92 mmol) successively and the reaction was stirred at 80° C. for 2 h. After completion of the reaction, the reaction mixture was diluted with water and extracted with ethyl acetate (×2). The combined organic layers were dried over MgSO₄, filtered and concentrated in vacuo. The solid was suspended in ether and then filtered and dried to afford compound 6 as an ivory solid (195 mg, 75%).

To a stirred solution of compound 6 (190 mg, 0.368 mmol) in DCM (5 mL) cooled to −10° C. was added DIBAL (1.84 mL, 1 M in THF, 5.0 eq) dropwise over a period of 15 min. After addition was complete, the reaction mixture was stirred at −10° C. for 2 h. The reaction mixture was slowly quenched with saturated NH₄Cl at −10° C. and extracted with ethyl acetate (×2). The combined organic layers were dried over MgSO₄, filtered and concentrated in vacuo and the residue was purified by column chromatography (SiO₂, EA/Hx=1/2) to afford an ivory solid. The solid was suspended in DCM, filtered and dried under vacuum to afford compound 7 (65 mg, 37%). LC-MS (ESI): m/z calcd for C₁₈H₁₂Cl₂F₃N₅O₃: 473.03, found: 474.1 [M+H]⁺.

### Example 2: Synthesis of (S)-3-(2,5-dichloro-4-(5-(6-(trifluoromethyl)imidazo[1,2-a]pyrazin-2-yl)-1,2,4-oxadiazol-3-yl)phenoxy)propane-1,2-diol (8)

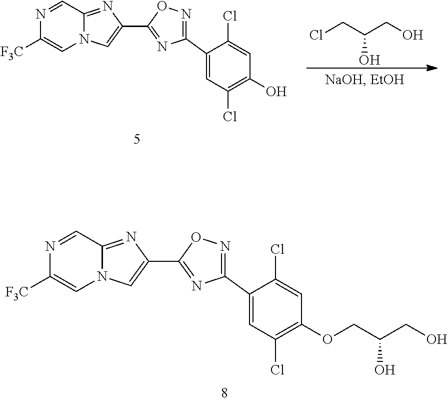 [SMILES: Oc1cc(Cl)c(-c2noc(-c3cn4cc(C(F)(F)F)ncc4n3)n2)cc1Cl ; OC[C@H](O)CCl ; OC[C@H](O)COc1cc(Cl)c(-c2noc(-c3cn4cc(C(F)(F)F)ncc4n3)n2)cc1Cl]

Compound 5 (1 g, 2.40 mmol, 1.0 eq) and sodium hydroxide (96 mg, 2.40 mmol, 1.0 eq) were combined in EtOH (10 mL) and stirred at room temperature for 10 min., then (S)-3-chloropropane-1,2-diol (292 mg, 2.64 mmol, 1.1 eq) was added. The reaction mixture was heated to 80° C. for 16 h. After completion, the reaction mixture was cooled, diluted with water (adding 1N HCl to acidify) and extracted with EA (×2). The combined organic layers were dried over MgSO₄, filtered and concentrated in vacuo. The residue was purified by column chromatography (SiO₂, DCM/MeOH=20/1) to afford a solid which was suspended in ether, filtered and dried under vacuum to afford compound 8 (220 mg, 18%). LC-MS (ESI): m/z calcd for C₁₈H₁₂Cl₂F₃N₅O₄: 489.02, found: 490.1 [M+H]⁺.

### Example 3: Synthesis (R)-3-(2,5-dichloro-4-(5-(6-(trifluoromethyl)imidazo[1,2-a]pyrazin-2-yl)-1,2,4-oxadiazol-3-yl)phenoxy)propane-1,2-diol (9)

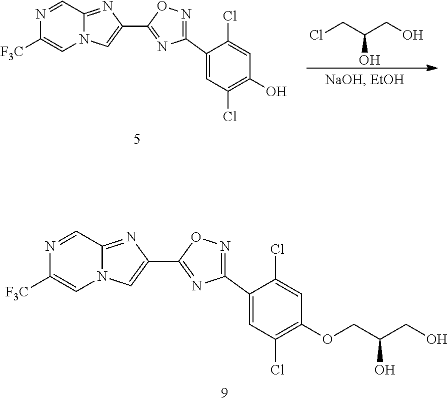 [SMILES: Oc1cc(Cl)c(-c2noc(-c3cn4cc(C(F)(F)F)ncc4n3)n2)cc1Cl ; OC[C@@H](O)CCl ; OC[C@@H](O)COc1cc(Cl)c(-c2noc(-c3cn4cc(C(F)(F)F)ncc4n3)n2)cc1Cl]

Compound 5 (300 mg, 0.72 mmol, 1.0 eq) and sodium hydroxide (28.8 mg, 0.72 mmol, 1.0 eq) were combined in EtOH (5 mL) and stirred at room temperature for 10 min., then (R)-3-chloropropane-1,2-diol (87 mg, 0.79 mmol, 1.1 eq) was added. The reaction mixture was heated to 80° C. for 16 h. After completion, the reaction mixture was cooled, diluted with water (adding 1N HCl to acidify) and extracted with EA (×2). The combined organic layers were dried over MgSO₄, filtered and concentrated in vacuo. The residue was purified by column chromatography (SiO₂, DCM/MeOH=20/1) to afford a solid which was suspended in ether, filtered and dried under vacuum to afford compound 9 (90 mg, 25%). LC-MS (ESI): m/z calcd for C₁₈H₁₂Cl₂F₃N₅O₄: 489.02, found: 490.2 [M+H]⁺.

### Example 4: Synthesis of(S)-2-amino-3-(2,5-dichloro-4-(5-(6-(trifluoromethyl)imidazo[1,2-b]pyridazin-2-yl)-1,2,4-oxadiazol-3-yl)phenoxy)propan-1-ol (19)

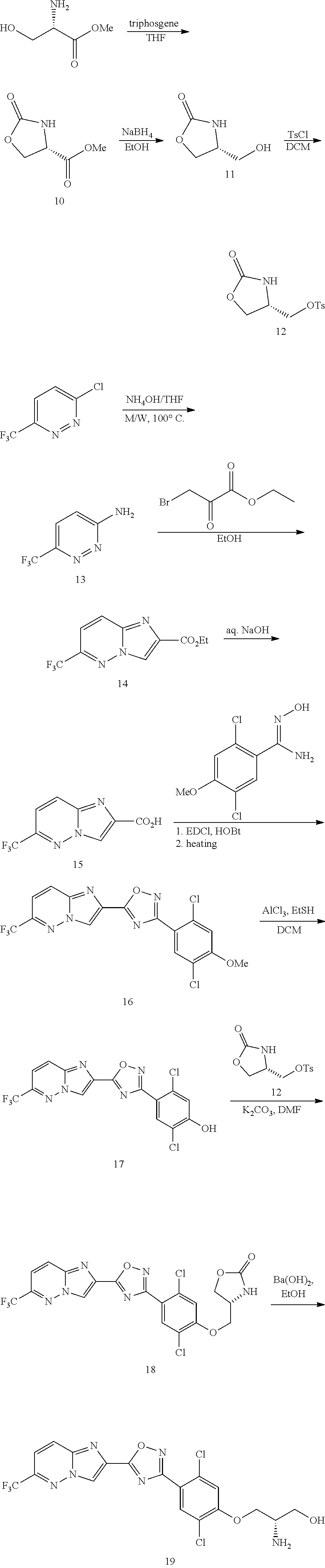

To a suspension of L-serine methyl ester HCl (6 g, 38.58 mmol) in THE (90 mL) was added THF (60 mL) solution of triphosgene (11.4 g, 38.58 mmol, 1.0 eq). The resulting mixture was refluxed for 4 h. Solvent was removed under reduced pressure. The residue was purified by flash chromatography on silica gel (EA:Hx=3:1) to afford compound 10 (4.38 g, 78%) as a solid.

A solution of the compound 10 (5.1 g, 35.14 mmol) in EtOH (100 mL) was cooled to −5 to 0° C. then NaBH₄ (1.59 g, 42.17 mmol, 1.2 eq) was added portionwise. The reaction was stirred for 3 h at room temperature and then treated with 10 mL of sat. aqueous NH₄Cl solution. The reaction was stirred for an additional 30 min and then white solid was filtered through a pad of celite. The filter cake was washed with EtOH. The filtrate was concentrated on a rotary evaporator. The residue was suspended in MeOH and the resulting solid was filtered. The filtrate was evaporated and dried under vacuum to afford compound 11 (4.7 g).

To a solution of the compound 11 (4.7 g) and p-toluenesulfonyl chloride (7.03 g, 36.89 mmol, 1.05 eq) in DCM (150 mL) were added DMAP (429 mg, 3.51 mmol, 0.1 eq) and TEA (5.9 mL, 42.17 mmol, 1.2 eq) at 0° C. The reaction mixture was allowed to warm to room temperature and stirred overnight. The reaction was quenched with water and the aqueous layer was extracted with DCM (×2). The combined organic layers were washed with 1N aqueous HCl (×2), dried over MgSO₄ and concentrated. The residue was purified by column chromatography (SiO₂, DCM/MeOH=20/1) to afford a solid. The solid was suspended in ether, filtered and dried under vacuum to afford compound 12 (3.3 g, 2 step yield 34%) as a white solid.

A mixture of 3-chloro-6-trifluoromethylpyridazine (1.6 g, 8.76 mmol) and ammonium hydroxide (12 mL) in THE (4 mL) was heated at 100° C. in a microwave reactor for 1 h. After cooling to room temperature, the reaction mixture was extracted with DCM (with MeOH 10%) and H₂O. The aqueous layer was extracted 5 times. The combined extracts were dried over with MgSO₄, filtered, and evaporated under reduce pressure to afford the amine compound 13 (1.37 g, 96%) as a white solid.

To a stirred solution of compound 13 (1.13 g, 6.93 mmol) in EtOH (20 mL) was added ethyl bromopyruvate (1.74 mL, 13.85 mmol, 2.0 eq) at room temperature. The resulting mixture was heated to 80° C. for 16 h. The reaction mixture was cooled to ambient temperature and concentrated. The residue was suspended in diethyl ether and the resulting solid was filtered and dried under vacuum to afford compound 14 (1.16 g, 64%) as a yellow solid.

To a stirred solution of compound 14 (1.16 g, 4.47 mmol) in MeOH (20 mL) was added 1N NaOH (10 mL). The mixture was stirred for 1 h. After completion, the reaction mixture was concentrated in vacuo, water was added to the residue and the mixture acidified to pH 2-3 using 1 N HCl. The resulting precipitate was filtered, washed with water, and dried to afford compound 15 (610 mg, 59%) as an ivory solid.

To a stirred solution of compound 15 (1.6 g, 6.92 mmol, 1.0 eq) in DMF (20 mL) were added EDCI (1.72 g, 8.99 mmol, 1.3 eq) and HOBt (1.21 g, 8.99 mmol, 1.3 eq). The mixture was stirred for 15 min. and hydroxyimidate (1.95 g, 8.30 mmol, 1.2 eq) was added. The reaction mixture was stirred at room temperature for 4 h and then the reaction mixture was stirred at 100° C. for 4 h. The reaction mixture was cooled to ambient temperature and extracted with EA (×2) and sat'd NH₄Cl solution. The combined organic layers were dried over MgSO₄, and concentrated in vacuo. The solid was suspended in IPA and then filtered and dried to afford compound 16 (1.67 g, 56%) as an orange solid.

To a cold solution of compound 16 (1.67 g, 3.88 mmol, 1.0 eq) in DCM (70 mL) was added AlCl₃ (2.58 g, 19.4 mmol, 5.0 eq) in small portions under Ar maintaining the temperature below 10° C. The light brown suspension was stirred for 10 min. and then EtSH (1.4 mL, 19.4 mmol, 5.0 eq) was added dropwise maintaining the temperature below 5° C. The reaction mixture was stirred for 3 h. The reaction was quenched with ice water and the biphasic mixture filtered and dried. The filtered aqueous layer was extracted with DCM/MeOH (9/1). The combined organic layers were dried over MgSO₄, and concentrated in vacuo. The combined solid was suspended in diethyl ether and then filtered and dried to afford compound 17 (1.32 g, 81%) as an ivory solid.

To a stirred solution of compound 17 (200 mg, 0.48 mmol, 1.0 eq) in DMF (5 mL) was added K₂CO₃ (133 mg, 0.96 mmol, 2.0 eq) followed by compound 12 (195 mg, 0.72 mmol, 1.5 eq) and the reaction was stirred at 80° C. for 3 h. After completion of the reaction by TLC, the reaction mixture was cooled to ambient temperature and extracted with EA (×2) and sat'd NH₄Cl solution. The combined organic layers were dried over MgSO₄, filtered and concentrated in vacuo. The residue was purified by column chromatography (SiO₂, DCM/MeOH=20/1) to afford compound 18 (167 mg, 67%).

To a stirred solution of the compound 18 (100 mg, 0.19 mmol, 1.0 eq) in EtOH/H₂O (30 mL/20 mL) was added Ba(OH)₂ 8H₂O (239 mg, 0.76 mmol, 4.0 eq) and the reaction was stirred at 70° C. for 1 h. After completion, the reaction mixture was cooled to ambient temperature and the solvent evaporated. The combined solid was slurried in water and then filtered and dried in vacuo. The residue was purified by column chromatography (SiO₂, DCM/MeOH=20/1 to 9/1) and the combined solid was slurried in diethyl ether and then filtered and dried to afford compound 19 (53 mg, 57%) as an ivory solid. LC-MS (ESI): m/z calcd for C₁₈H₁₃Cl₂F₃N₆O₃: 488.04, found: 489.1 [M+H]⁺.

### Example 5: Synthesis of 2-(2,5-dichloro-4-(5-(6-(trifluoromethyl)imidazo[1,2-b]pyridazin-2-yl)-1,2,4-oxadiazol-3-yl)phenoxy)propan-1-ol (21)

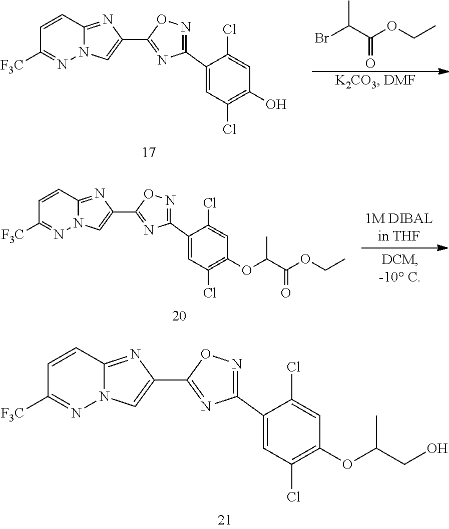

To a stirred solution of compound 17 (300 mg, 0.72 mmol, 1.0 eq) in DMF (5 mL) was added K₂CO₃ (398 mg, 2.88 mmol, 4.0 eq) followed by ethyl 2-bromopropionate (0.37 mL, 2.88 mmol, 4.0 eq). The reaction was stirred at 80° C. for 2 h. After completion, the reaction mixture was cooled, diluted with EA (×2) and extracted with sat'd NH₄Cl solution. The combined organic layers were dried over MgSO₄, filtered and concentrated in vacuo. The solid was suspended in ether and then filtered and dried to afford compound 20 (300 mg, 80%).

To a stirred solution of compound 20 (270 mg, 0.523 mmol, 1.0 eq) in DCM (5 mL) cooled to −10° C. was added DIBAL (2.6 mL, 1 M in THF, 5.0 eq) dropwise over a period of 15 min. After addition was complete, the reaction mixture was stirred at −10° C. for 2 h. After completion, the reaction mixture was slowly quenched with saturated NH₄Cl at −10° C. The reaction mixture was extracted with EA (×2) and the combined organic layers were dried over MgSO₄, filtered and concentrated in vacuo. The solid was suspended in ether, filtered and dried under vacuum to afford compound 21 (200 mg, 80%). LC-MS (ESI): m/z calcd for C₁₈H₁₂Cl₂F₃N₅O₃: 473.03, found: 474.1 [M+H]⁺.

### Example 6: Synthesis of (S)-3-(2,5-dichloro-4-(5-(6-(trifluoromethyl)imidazo[1,2-b]pyridazin-2-yl)-1,2,4-oxadiazol-3-yl)phenoxy)propane-1,2-diol (22)

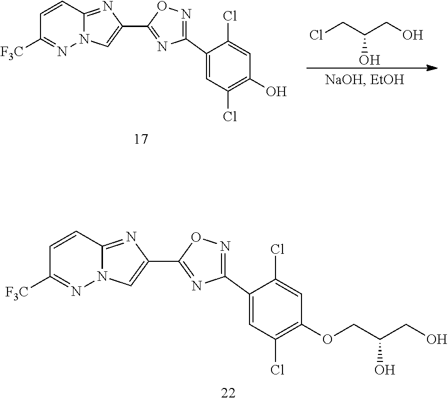 [SMILES: Oc1cc(Cl)c(-c2noc(-c3cn4nc(C(F)(F)F)ccc4n3)n2)cc1Cl ; OC[C@H](O)CCl ; OC[C@H](O)COc1cc(Cl)c(-c2noc(-c3cn4nc(C(F)(F)F)ccc4n3)n2)cc1Cl]

Compound 17 (200 mg, 0.48 mmol, 1.0 eq) and sodium hydroxide (28.8 mg, 0.72 mmol, 1.5 eq) were combined in EtOH (5 mL) and stirred at room temperature for 1 h, then (S)-3-chloropropane-1,2-diol (58 mg, 0.528 mmol, 1.1 eq) was added. The reaction mixture was heated to 80° C. for 16 h. After completion, the reaction mixture was diluted with water (adding 1N HCl to acidify) and extracted with EA (×2). The combined organic layers were dried over MgSO₄, filtered and concentrated in vacuo. The residue was purified by column chromatography (SiO₂, DCM/MeOH=20/1) to afford a solid which was suspended in ether, filtered and dried under vacuum to afford compound 22 (120 mg, 51%). LC-MS (ESI): m/z calcd for C₁₈H₁₂Cl₂F₃N₅O₄: 489.02, found: 490.1 [M+H]⁺.

### Example 7: Synthesis of(S)-2-amino-3-(2,5-dichloro-4-(5-(6-(trifluoromethyl)imidazo[1,2-a]pyrazin-2-yl)-1,2,4-oxadiazol-3-yl)phenoxy)propan-1-ol (24)

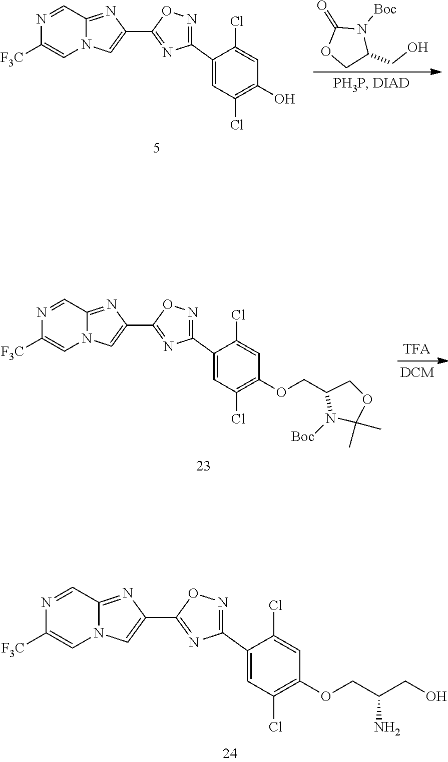 [SMILES: Oc1cc(Cl)c(-c2noc(-c3cn4cc(C(F)(F)F)ncc4n3)n2)cc1Cl ; CC(C)(C)OC(=O)N1C(=O)OC[C@H]1CO ; CC(C)(C)OC(=O)N1[C@H](COc2cc(Cl)c(-c3noc(-c4cn5cc(C(F)(F)F)ncc5n4)n3)cc2Cl)COC1(C)C ; N[C@@H](CO)COc1cc(Cl)c(-c2noc(-c3cn4cc(C(F)(F)F)ncc4n3)n2)cc1Cl]

To a stirring solution of compound 5 (200 mg, 0.48 mmol, 1.0 eq) and the N-Boc protected alcohol (133 mg, 0.576 mmol, 1.2 eq) in THF (5 mL) was added Ph₃P (188 mg, 0.72 mmol, 1.5 eq). The reaction mixture was cooled to 0° C. and DIAD (0.142 mL, 0.72 mmol, 1.5 eq) was added dropwise to the reaction mixture. The reaction mixture was stirred at rt for 48 h. The reaction mixture was extracted with water and EA (×2). The combined organic layers were dried over MgSO₄, filtered and concentrated in vacuo. The residue was purified by column chromatography (SiO₂, EA/Hx=1/4 to 1/2) to afford compound 23 (270 mg, 89%).

To a stirring solution of compound 23 (270 mg, 0.428 mmol, 1.0 eq) in DCM (10 mL) cooled to 0° C. was added TFA (5 mL). The reaction mixture was stirred at room temperature for 2 h. After completion, the reaction mixture was concentrated in vacuo, the residue was extracted with sat'd NaHCO₃ and DCM (with 10% MeOH) (×4). The combined organic layers were dried over MgSO₄, filtered and concentrated in vacuo. The residue was purified by column chromatography (SiO₂, DCM/MeOH=9/1) and the combined solid was slurried in diethyl ether and then filtered and dried to afford compound 24 (47 mg, 22%). LC-MS (ESI): m/z calcd for C₁₈H₁₃Cl₂F₃N₆O₃: 488.04, found: 489.1 [M+H]⁺.

### Example 8: Synthesis of (S)-2-(2,5-dichloro-4-(5-(6-(trifluoromethyl)imidazo[1,2-a]pyrazin-2-yl)-1,2,4-oxadiazol-3-yl)phenoxy)propan-1-ol (25)

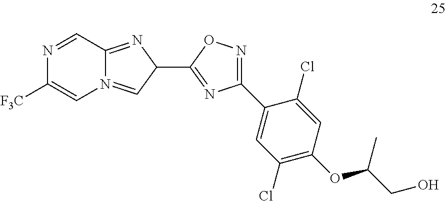

Compound 25 was prepared in a similar manner as described above. LC-MS (ESI): m/z calcd for C₁₈H₁₂Cl₂F₃N₅O₃: 473.03, found: 474.2 [M+H]⁺.

### Example 9: Synthesis of 2,5-dichloro-4-(5-(8-chloro-6-(trifluoromethyl)imidazo[1,2-a]pyridin-2-yl)-1,2,4-oxadiazol-3-yl)-N-(5-oxopyrrolidin-3-yl)benzamide (38)

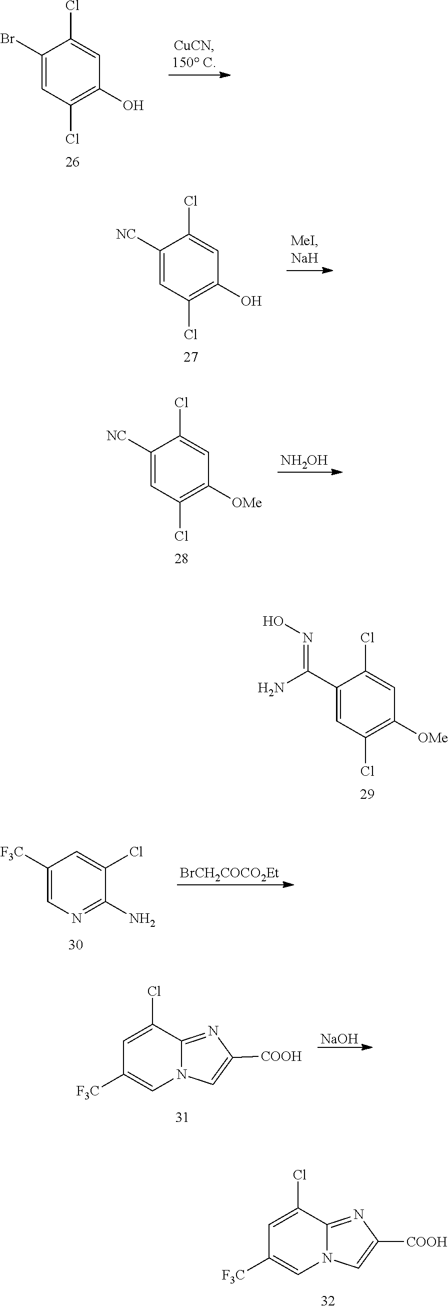

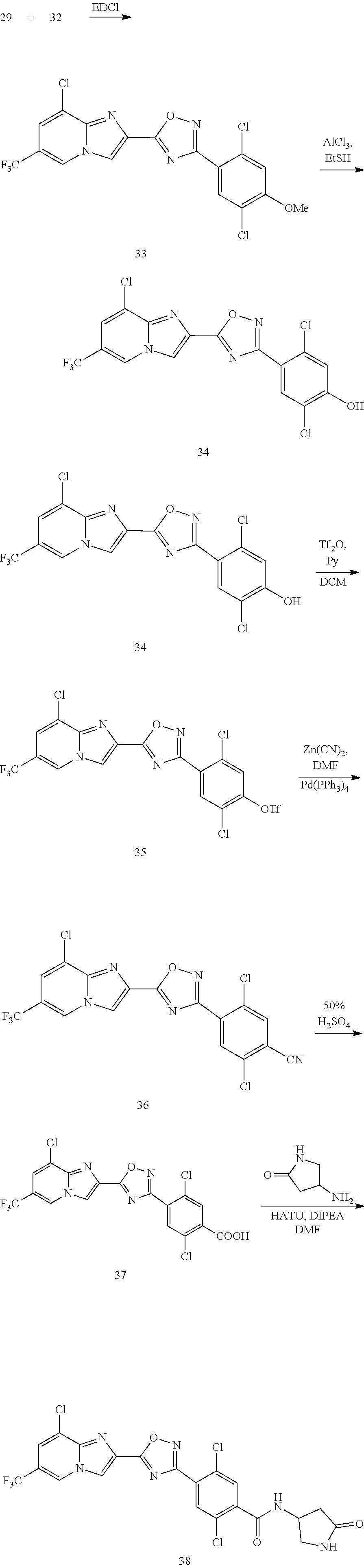

To a stirred solution of 2,5-dichloro-4-bromophenol (26) (210.0 g, 0.86 mol) in DMF (1000 mL) was added cuprous cyanide (101.5 g, 1.13 mmol) at room temperature. The reaction mixture was stirred at 150° C. for 4 h. The mixture was concentrated under vacuum. Water and EtOAc were added to the residue and then filtered through a pad of celite. The filtrate was extracted with EtOAc and the combined organic layers were dried over anhydrous Na₂SO₄ and concentrated under vacuum. The residue was recrystallized from petroleum ether/EtOAc (10:1, 1400 mL) to afford 2,5-dichloro-4-hydroxybenzonitrile (27) (93.0 g, 57%) as a white solid.

To a stirred solution of 2,5-dichloro-4-hydroxybenzonitrile (27) (35.0 g, 186 mmol) in DMF (150 mL) was added NaH (13.7 g, 347 mmol) in small portions at 0° C. and the mixture was stirred for 30 min at 0° C. Methyl iodide (35 mL, 560 mmol) was added dropwise and the reaction mixture was allowed to warm to room temperature and stirred for 3 h. The mixture was cooled to 0° C. and ice-water was added carefully. The resulting precipitate was collected by filtration, washed with water and dried to afford compound (28) (29 g, 78%) as a white solid.

To a stirred solution of hydroxylamine hydrochloride (64.0 g, 0.5 mol) in EtOH (500 mL) was added triethylamine (160.0 g, 1.27 mol) and the mixture was stirred for 30 min at room temperature. Compound (28) was added and the reaction mixture was stirred at 80° C. for 4 h. The mixture was concentrated and the residue was dissolved in EtOAc. The resulting solution was washed with water, dried over anhydrous Na₂SO₄ and concentrated under vacuum to afford a mixture (60.0 g, compound (29) and 2,5-dichloro-4-methoxybenzamide, 1:2) as an off-white solid. The solid was slurried in MBTE and then filtered. The filtrate was concentrated under vacuum to afford a solid (40.1 g, 28%, compound (29) and 2,5-dichloro-4-methoxybenzamide, 1:1).

To a stirred solution of 2-amino-3-chloro-5-trifluoromethylpyridine (30) (50.0 g, 0.25 mmol) in EtOH (500 mL) was added ethylbromopyruvate (80.0 mL, 0.64 mol) at room temperature. The reaction mixture was heated at 80° C. for 48 h and then cooled to room temperature. The mixture was concentrated and the residue was suspended in diethyl ether. The resulting precipitate was collected by filtration and dried under vacuum to afford ethyl 8-chloro-6-(trifluoromethyl)imidazo[1,2-a]pyridine-2-carboxylate (31) (64.0 g, 86%) as an off-white solid.

To a stirred solution of ethyl 8-chloro-6-(trifluoromethyl)imidazo[1,2-a]pyridine-2-carboxylate (31) (64.0 g, 0.22 mol) in MeOH (64.0 mL) was added 1M aqueous NaOH (640.0 mL). The reaction mixture was heated at 50° C. for 1 h and then cooled to room temperature. The mixture was concentrated under vacuum. Water was added to the residue and the mixture was acidified to pH=4 with AcOH. The resulting precipitate was collected by filtration, washed with water and dried under vacuum to afford compound (32) (24.0 g) as an off-white solid. The filtrate was extracted with EtOAc and the combined organic layers were dried over anhydrous Na₂SO₄ and concentrated under vacuum to afford another portion of compound (32) (20.0 g) as an off-white solid (combined yield 77%).

To a stirred solution of compound (32) (26.5 g, 100 mmol) in DMF (50.0 mL) were added EDCI-HCl (19.2 g, 100 mmol) and HOBt (13.5 g, 100 mmol). The mixture was stirred for 15 min and hydroxyimidate (29) (36 g, ˜54% purity, 100 mmol) was added. The reaction mixture was stirred at 100° C. for 12 h. The mixture was concentrated under vacuum and the residue was purified by flash column chromatography on silica gel (petroleum ether/EtOAc=10:1) to afford compound (33) (12.6 g, 33%) as a white solid.

To a cold solution of compound (33) (16 g, 34.5 mmol) in DCM (110 mL) was added AlCl₃ (23 g, 172.5 mmol) in small portions under N₂ maintaining the temperature below 10° C. The light brown suspension was stirred for 10 min and then EtSH (12.8 mL, 172.5 mmol) was added dropwise maintaining the temperature below 5° C. The reaction mixture was stirred for 2.5 h at below 10° C. and then slowly poured into ice-water with strong agitation. The organic layer was separated and the aqueous layer was extracted with DCM. The combined DCM layers were washed with water, dried over anhydrous Na₂SO₄ and concentrated. The residue was azeotroped with toluene to afford compound (34) (15.5 g, 100%) as an off-white solid.

To a dispersion of compound (34) (3 g, 6.673 mmol) in DCM (200 mL) were added pyridine (2.7 mL, 33.3 mmol, 5 eq) and Tf₂O (1.68 mL, 10.02 mmol, 1.5 eq) at 0° C. The reaction mixture was allowed to warm to room temperature and stirred overnight. The reaction was quenched with water and the aqueous layer was extracted with DCM (X₂). The combined organics were dried over MgSO₄ and concentrated to afford compound (35) (3.87 g, 100%) as a white solid.

To a stirred solution of compound (35) (1 g, 1.72 mmol) in DMF (20 mL) were added zinc cyanide (303 mg, 2.58 mmol, 1.5 eq) and [Pd(PPh₃)₄] (198 mg, 0.172 mmol, 0.1 eq) The solution was degassed with Ar for 10 min and the reaction mixture was heated at 80° C. for 15 h. After completion of the reaction, the reaction mixture was diluted with sat'd NH₄Cl solution and extracted with EA (×2). The combined organic layers were dried over MgSO₄, filtered and concentrated in vacuo. The resulting solid was suspended in ether, filtered and dried to afford compound (36) (315 mg, 40%) as an ivory solid.

50% H₂SO₄ aq. solution was carefully added to the compound (36) (315 mg, 0.687 mmol) and the resulting solution was refluxed for 6 h. After cooling to room temperature, the reaction mixture was diluted with water and the resulting solid was filtered, washed with water and hexanes and dried in vacuo to afford compound (37) (200 mg, 61%, ivory solid).

To a stirred solution of compound (37) (200 mg, 0.418 mmol) in DMF (5 mL) were added HATU (376 mg, 0.627 mmol, 1.5 eq) and DIPEA (0.23 mL, 1.25 mmol, 3.0 eq). The mixture was stirred for 10 min and 4-aminopyrrolidin-2-one (52 mg, 0.502 mmol, 1.2 eq) was added. The reaction mixture was stirred at room temperature overnight. The reaction mixture was diluted with sat'd NH₄Cl solution and extracted with EA (×2). The combined organic layers were dried over MgSO₄, filtered and concentrated in vacuo. The crude compound purified by column chromatography (SiO₂, DCM/MeOH=20/1) to afford compound (38) (120 mg, 51%) as a white solid. LC-MS (ESI): m/z calcd for C₂₁H₁₂C₃F₃N₆O₃: 559.00, found: 559.2 [M+H]⁺.

### Example 10: Synthesis of 2-((4-(5-(6-(trifluoromethyl)imidazo[1,2-b]pyridazin-2-yl)-1,2,4-oxadiazol-3-yl)-2,3-dihydro-1H-inden-1-yl)amino)ethan-1-ol (45)

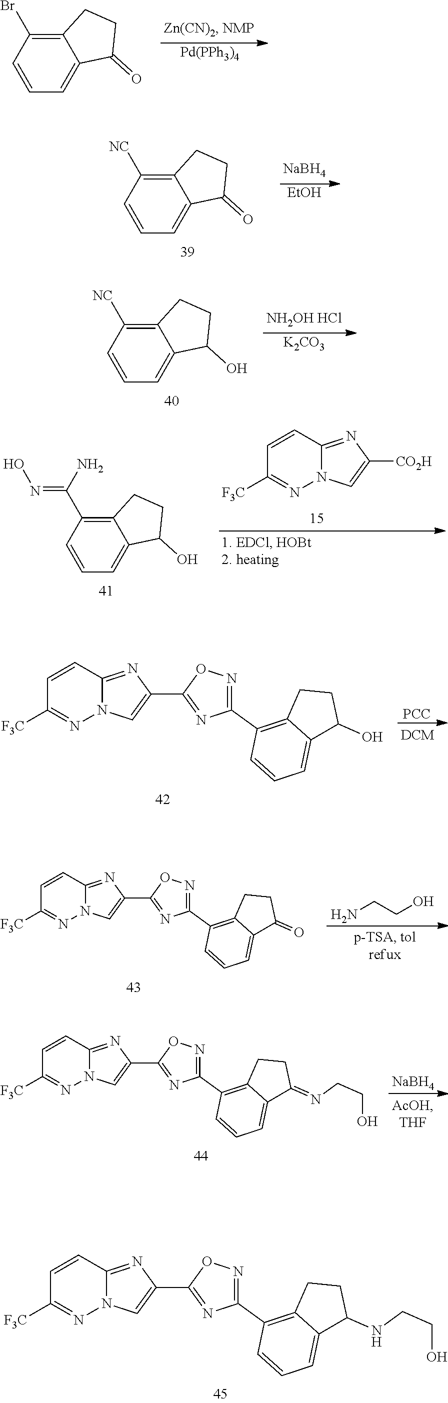

To a stirred solution of 4-bromo-1-indanone (10 g, 47.37 mmol) in NMP (30 mL) were added zinc cyanide (11.12 g, 94.74 mmol, 2.0 eq) and [Pd(PPh₃)₄] (2.7 g, 2.37 mmol, 0.05 eq). The solution was degassed with Ar for 10 min and the reaction mixture was heated at 100° C. for 7 h. Upon cooling, the reaction mixture was poured onto ice water. The compound and inorganic Zn salts precipitated. The solid was collected and partitioned between DCM (×3) and water. The organic layers were filtered to remove the Zn salts, and the filtrate was concentrated and precipitated from a 4:1 mixture of EtOH and MeOH to give 3.7 g (49%) of compound 39 as a yellow solid.

To a stirred suspension of the compound 39 (3.8 g, 24.2 mmol) and silica gel (cat.) in EtOH (15 mL) at 0° C. was added NaBH₄ (457 mg, 12.1 mmol, 0.5 eq). The reaction was allowed to warm up to room temperature and stirred for 2 h. The solvent was removed under reduced pressure and the product purified by column chromatography (SiO₂, DCM/EA=2/1 to 1/1) to afford compound 40 (3.3 g, 86%).

To a stirred solution of the compound 40 (500 mg, 3.14 mmol) in EtOH (20 mL) were added cautiously over a period of 16 h under refluxing condition hydroxylamine hydrochloride (1.09 g, 15.7 mmol, 5.0 eq) and K₂CO₃ (2.17 g, 15.7 mmol, 5.0 eq) equal portions. The mixture was cooled to room temperature and the solid was filtered. The organic solvent was concentrated under reduced pressure and the product purified by column chromatography (SiO₂, DCM/MeOH=9/1) to afford compound 41 (520 mg, 86%).

To a stirred solution compound 15 (370 mg, 1.6 mmol) in DMF (5 mL) were added EDCI (399 mg, 2.08 mmol, 1.3 eq) and HOBt (281 mg, 2.08 mmol, 1.3 eq). The mixture was stirred for 15 min and hydroxyimidate compound 41 (399 mg, 2.08 mmol, 1.3 eq) was added. The reaction mixture was stirred at room temperature for 2 h and then the reaction mixture was stirred at 120° C. for 3 h. The reaction mixture was cooled to ambient temperature and extracted with brine and EA (×3). The combined organic layers were dried over MgSO₄, filtered and concentrated in vacuo. The residue was purified by column chromatography (SiO₂, DCM/MeOH=9/1) to afford the compound 42 (480 mg, 79%).

To a stirred solution of the compound 42 (480 mg, 1.24 mmol) in DCM (5 mL) were added molecular sieve (one-half the weight of PCC) and pyridinium chlorochromate (400 mg, 1.85 mmol, 1.5 eq) at 0° C. The reaction was allowed to warm up to room temperature and stirred for 7 h. After completion of the reaction, the reaction mixture was filtered through a celite pad. The solvent was removed under reduced pressure and the product was purified by column chromatography (SiO₂, DCM/MeOH=20/1) to afford compound 43 (430 mg, 90%).

To To a stirred solution of the compound 43 (330 mg, 0.856 mmol) and 2-aminoethanol (157 mg, 2.56 mmol, 3.0 eq) in toluene (5 mL) was added p-toluenesulfonic acid monohydrate (32.5 mg, 0.17 mmol, 0.2 eq). The reaction was heated to reflux under an atmosphere of Ar for 2 h. At this point, the mixture was cooled and diluted with toluene. The mixture was washed with sat'd NaHCO₃ solution and water. The organic layer was concentrated in vacuo and the residue (compound 44) was dissolved in THE (10 mL) and cooled to 0° C., acetic acid (0.17 mL, 2.99 mmol, 3.5 eq) was added, followed by the addition of NaBH₄ (37 mg, 0.98 mmol, 1.15 eq). The reaction was allowed to warm to room temperature and stirred overnight. The mixture was partitioned between EA (×2) and sat'd NaHCO₃ solution. The combined organic layers were dried over MgSO₄, filtered and concentrated in vacuo. The residue was purified by column chromatography (SiO₂, DCM/MeOH=9/1). The solid was suspended in ether, filtered and dried under vacuum to afford compound 45 (67 mg, 18%). LC-MS (ESI): m/z calcd for C₂₀H₁₇F₃N₆O₂: 431.14, found: 431.3 [M+H].

### Example 11: GTPγS Binding Assay

S1P1 membrane is prepared from CHO-K1 Gαqi5 cells expression full-length human S1P1. Scintillation proximity assay (SPA) is performed by incubating membranes, GTPγ³⁵S, and compounds at various concentrations for 60 minutes. Wheat germ agglutinin-coated SPA beads are added and incubated for 60 minutes before centrifugation and scintillation counting. Data are shown below in Table 1.

TABLE 1

| Compound | EC50 (μM) |
|---|---|
| 7 | A |
| 8 | A |
| 9 | A |
| 19 | A |
| 21 | B |
| 22 | B |
| 24 | A |
| 25 | NT |
| 38 | NT |
| 45 | NT |

A, EC₅₀ < 100 nM; B, EC₅₀ = 100 nM-1 μM; NT = not tested

### Example 12: Phase 3 Study to Evaluate Safety and Efficacy of a Compound of Formula (I), (Ia), (b), (Ic), (Id), (II), (IIa), (IIb), (IIc), (IId), (III), (IIIa), (IIIb), (IIc), (IId), or (IIIe) in Patients with Relapsing Multiple Sclerosis (MS)

The primary objective of this study is to assess tolerability and safety and health outcomes in relapsing MS patients taking a compound of Formula (I), (Ia), (Ib), (Ic), (Id), (II), (IIa), (IIb), (IIc), (IId), (III), (IIIa), (IIIb), (IIIc), (IIId), or (IIIe).

Patients: Eligible patients will be men and women 18 years to 65 years of age.

Criteria:

Inclusion Criteria:
Patients is 18-65 years of age, must have relapsing MS

Exclusion Criteria:
Patients with a type of MS that is not relapsing
Patients with history of chronic immune disease
Patients with a history of certain cancers
Diabetic patients with certain eye disorders
Patients who are on certain immunosuppressive medications or heart medications
Patients with certain heart conditions
Patients with certain lung conditions

Study Type: Interventional

Study Design: Intervention Model: Single Group Assignment
Masking: Open Label
Primary Purpose: Treatment

Primary Outcome Measures:
The primary objective of this study is to evaluate the safety and tolerability profile of a compound of Formula (I), (Ia), (Ib), (Ic), (Id), (II), (IIa), (IIb), (IIc), (IId), (III), (IIIa), (IIIb), (IIIc), (IIId), or (IIIe) in patients with relapsing forms of MS.

Secondary Outcome Measures:
Incidence of macular edema.
Incidence of bradyarrhythmic electrocardiograms.
Patient reported outcomes indices in multiple sclerosis (PRIMUS), short form health survey-12, and treatment satisfaction questionnaire for medication.

| Condition | Intervention |
|---|---|
| Relapsing Multiple | Compound of Formula (I), (Ia), (Ib), (Ic), |
| Sclerosis | (Id), (II), (IIa), (IIb), (IIc), (IId), (III), (IIIa), (IIIb), |
| | (IIIc), (IIId), or (IIIe) |

What is claimed is:

1. A compound having the structure of Formula (I), or a pharmaceutically acceptable salt or solvate thereof: 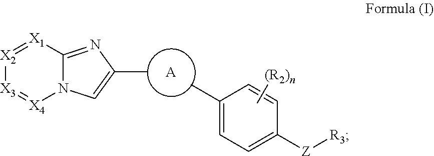
wherein:
X₁ is N; X₂, X₃, and X₄ are each CR₁; or
X₂ is N; X₁, X₃, and X₄ are each CR₁; or
X₃ is N; X₁, X₂, and X₄ are each CR₁; or
X₄ is N; X₁, X₂, and X₃ are each CR₁; 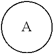
is selected from 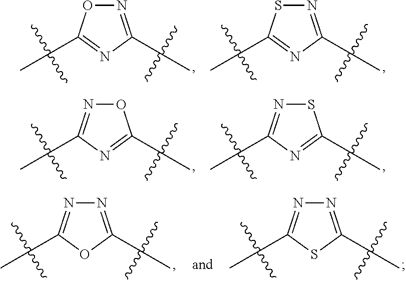 [SMILES: *c1noc(*)n1 ; *c1nsc(*)n1 ; *c1noc(*)n1 ; *c1nsc(*)n1 ; *c1nnc(*)o1 ; *c1nnc(*)s1]
Z is —O—, —S—, —N(R₄)—, or —CH₂—;
each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₁₂, —N(R₁₃)N(R₁₁)S(O)₂R₁₅, —C(O)R₁₄, —C(O)OR₁₀, —C(S)OR₁₀, —C(O)SR₁₀, —C(O)N(R₁₁)R₁₂, —C(S)N(R₁₁)R₁₂, —C(O)N(R₁₁)S(O)₂R₁₅, —C(S)N(R₁₁)S(O)₂R₁₅, —C(O)N(R₁₃)N(R₁₁)R₁₂, —C(S)N(R₁₃)N(R₁₁)R₁₂, and —C(O)N(R₁₃)N(R₁₁)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₈cycloalkyl, C₁-C₆alkoxy, C₂-C₉heterocycloalkyl, phenyl, and heteroaryl;
R₄ is hydrogen or optionally substituted C₁-C₆alkyl;
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring; and
n is 0-4.

2. A compound having the structure of Formula (II), or a pharmaceutically acceptable salt or solvate thereof: 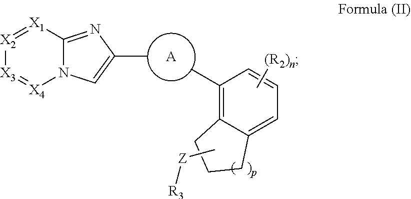
wherein:
X₁ is N; X₂, X₃, and X₄ are each CR₁; or
X₂ is N; X₁, X₃, and X₄ are each CR₁; or
X₃ is N; X₁, X₂, and X₄ are each CR₁; or
X₄ is N; X₁, X₂, and X₃ are each CR₁; 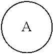
is selected from 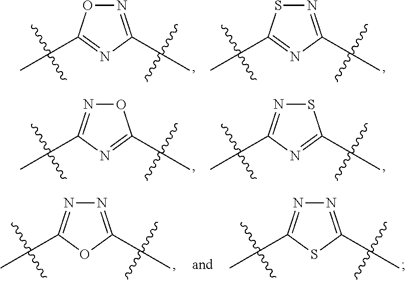 [SMILES: *c1noc(*)n1 ; *c1nsc(*)n1 ; *c1noc(*)n1 ; *c1nsc(*)n1 ; *c1nnc(*)o1 ; *c1nnc(*)s1]
Z is —O—, —S—, —N(R₄)—, or —CH₂—;
each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₁₂, —N(R₁₃)N(R₁₁)S(O)₂R₁₅, —C(O)R₁₄, —C(O)OR₁₀, —C(S)OR₁₀, —C(O)SR₁₀, —C(O)N(R₁₁)R₁₂, —C(S)N(R₁₁)R₁₂, —C(O)N(R₁₁)S(O)₂R₁₅, —C(S)N(R₁₁)S(O)₂R₁₅, —C(O)N(R₁₃)N(R₁₁)R₁₂, —C(S)N(R₁₃)N(R₁₁)R₁₂, and —C(O)N(R₁₃)N(R₁₁)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is C₁-C₆alkyl substituted with one, two, or three groups selected from halogen, —CN, —NH₂, —NH(C₁-C₆alkyl), —NH(C₁-C₆alkyl-OH), —N(C₁-C₆alkyl)₂, —N(C₁-C₆alkyl-OH)₂, —NHCH(C₁-C₆alkyl-OH)₂, —OH, —CO₂H, —CO₂C₁-C₆alkyl, —C(═O)C₁-C₆alkyl, —C(═O)C₁-C₆haloalkyl, —C(═O)NH₂, —C(═O)NH(C₁-C₆alkyl), —C(═O)NC₁-C₆alkyl)₂, —S(═O)₂NH₂, —S(═O)₂NH(C₁-C₆alkyl), —S(═O)₂N(C₁-C₆alkyl)₂, C₃-C₆cycloalkyl, C₁-C₆alkoxy, C₂-C₉heterocycloalkyl, phenyl, and heteroaryl;
R₄ is hydrogen or optionally substituted C₁-C₆alkyl;
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring;
n is 0-3, and
p is 1 or 2.

3. The compound of claim 2, or a pharmaceutically acceptable salt or solvate thereof, wherein p is 1.

4. The compound of claim 2, or a pharmaceutically acceptable salt or solvate thereof, wherein p is 2.

5. The compound of any one of claims 1-4, or a pharmaceutically acceptable salt or solvate thereof, wherein Z is —O—.

6. The compound of any one of claims 1-4, or a pharmaceutically acceptable salt or solvate thereof, wherein Z is —N(H)—.

7. The compound of any one of claims 1-6, or a pharmaceutically acceptable salt or solvate thereof, wherein R₃ is C₁-C₆alkyl substituted one, two, or three groups selected from halogen, —CN, —NH₂, —NH(C₁-C₆alkyl), —N(C₁-C₆alkyl)₂, —OH, —CO₂H, —CO₂C₁-C₆alkyl, —C(═O)NH₂, —C(═O)NH(C₁-C₆alkyl), and —C(═O)N(alkyl)₂.

8. The compound of any one of claims 1-7, or a pharmaceutically acceptable salt or solvate thereof, wherein R₃ is C₁-C₆alkyl substituted with one or two —OH.

9. A compound having the structure of Formula (III), or a pharmaceutically acceptable salt or solvate thereof: 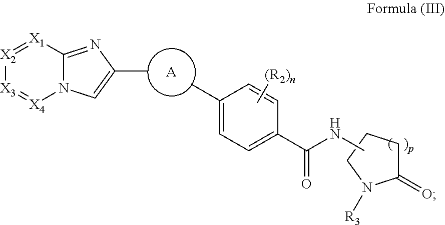
wherein:
X₁, X₂, X₃, and X₄ are each CR₁; or
X₁ is N; X₂, X₃, and X₄ are each CR₁; or
X₂ is N; X₁, X₃, and X₄ are each CR₁; or
X₃ is N; X₁, X₂, and X₄ are each CR₁; or
X₄ is N; X₁, X₂, and X₃ are each CR₁; 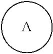
is selected from 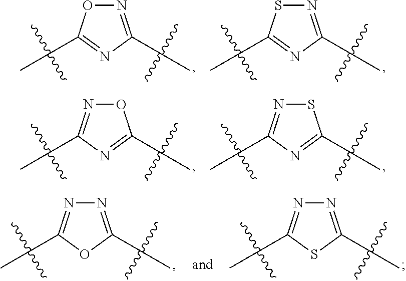 [SMILES: *c1noc(*)n1 ; *c1nsc(*)n1 ; *c1noc(*)n1 ; *c1nsc(*)n1 ; *c1nnc(*)o1 ; *c1nnc(*)s1]
each R₁ is independently selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —CF₃, —OR₁₀, —SR₁₀, —N(R₁₁)R₁₂, —N(R₁₁)S(O)₂R₁₅; —N(R₁₃)N(R₁₁)R₁₂, —N(R₁₃)N(R₁₁)S(O)₂R₁₅, —C(O)R₁₄, —C(O)OR₁₀, —C(S)OR₁₀, —C(O)SR₁₀, —C(O)N(R₁₁)R₁₂, —C(S)N(R₁₁)R₁₂, —C(O)N(R₁₁)S(O)₂R₁₅, —C(S)N(R₁₁)S(O)₂R₁₅, —C(O)N(R₁₃)N(R₁₁)R₁₂, —C(S)N(R₁₃)N(R₁₁)R₁₂, and —C(O)N(R₁₃)N(R₁₁)S(O)₂R₁₅;
each R₂ is independently selected from the group consisting of halogen, optionally substituted C₁-C₆alkyl, —OR₂₀, —SR₂₀, —N(R₂₁)R₂₂, —C(O)R₂₀, —C(O)N(R₂₁)R₂₂, and —N(R₂₃)C(O)R₂₀;
R₃ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, and optionally substituted —(C₁-C₂alkylene)-(heteroaryl);
R₁₀, R₁₃ and R₁₄ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₁₁ and R₁₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₁₁ and R₁₂ together with the nitrogen atom to which they are attached, form an optionally substituted C₂-C₉heterocycloalkyl ring;
R₁₅ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₀ and R₂₃ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R₂₁ and R₂₂ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R₂₁ and R₂₂ together with the nitrogen atom to which they are attached, form an optionally substituted C₂-C₉heterocycloalkyl ring;
n is 0-4; and
p is 1 or 2.

10. The compound of claim 9, or a pharmaceutically acceptable salt or solvate thereof, wherein p is 1.

11. The compound of claim 9, or a pharmaceutically acceptable salt or solvate thereof, wherein p is 2.

12. The compound of any one of claims 9-11, or a pharmaceutically acceptable salt or solvate thereof, wherein R₃ is hydrogen or C₁-C₆alkyl.

13. The compound of claim 12, or a pharmaceutically acceptable salt or solvate thereof, wherein R₃ is hydrogen.

14. The compound of any one of claims 9-13, or a pharmaceutically acceptable salt or solvate thereof, wherein X₁, X₂, X₃, and X₄ are each CR₁.

15. The compound of any one of claims 1-13, or a pharmaceutically acceptable salt or solvate thereof, wherein X₁ is N; X₂, X₃, and X₄ are each CR₁.

16. The compound of any one of claims 1-13, or a pharmaceutically acceptable salt or solvate thereof, wherein X₂ is N; X₁, X₃, and X₄ are each CR₁.

17. The compound of any one of claims 1-13, or a pharmaceutically acceptable salt or solvate thereof, wherein X₃ is N; X₁, X₂, and X₄ are each CR₁.

18. The compound of any one of claims 1-13, or a pharmaceutically acceptable salt or solvate thereof, wherein X₄ is N; X₁, X₂, and X₃ are each CR₁.

19. The compound of anyone of claims 1-18, or a pharmaceutically acceptable salt or solvate thereof, wherein each R₁ is independently selected from the group consisting of hydrogen, halogen, optionally substituted C₁-C₆alkyl, —CF₃, —OR₁₀, —N(R₁₁)R₁₂, —C(O)R₁₄, —C(O)OR₁₀, and —C(O)N(R₁₁)R₁₂.

20. The compound of anyone of claims 1-19, or a pharmaceutically acceptable salt or solvate thereof, wherein each R₁ is independently selected from the group consisting of hydrogen, halogen, and —CF₃.

21. The compound of anyone of claims 1-20, or a pharmaceutically acceptable salt or solvate thereof, wherein

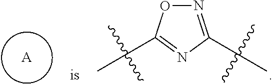

22. The compound of any one of claims 1-20, or a pharmaceutically acceptable salt or solvate thereof, wherein

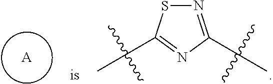

23. The compound of any one of claims 1-20, or a pharmaceutically acceptable salt or solvate thereof, wherein

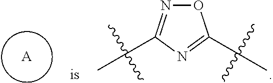

24. The compound of any one of claims 1-20, or a pharmaceutically acceptable salt or solvate thereof, wherein

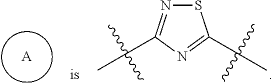

25. The compound of any one of claims 1-20, or a pharmaceutically acceptable salt or solvate thereof, wherein

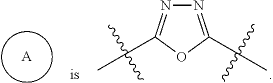

26. The compound of any one of claims 1-20, or a pharmaceutically acceptable salt or solvate thereof, wherein

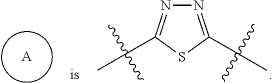

27. The compound of any one of claims 1-26, or a pharmaceutically acceptable salt or solvate thereof, wherein each R₂ is independently selected from the group consisting of halogen, optionally substituted C₁-C₆alkyl, —OR₂₀, and —N(R₂₁)R₂₂.

28. The compound of any one of claims 1-27, or a pharmaceutically acceptable salt or solvate thereof, wherein each R₂ is independently selected from the group consisting of halogen and C₁-C₆alkyl.

29. The compound of any one of claims 1-26, or a pharmaceutically acceptable salt or solvate thereof, wherein n is 0.

30. The compound of any one of claims 1-28, or a pharmaceutically acceptable salt or solvate thereof, wherein n is 1.

31. The compound of any one of claims 1-28, or a pharmaceutically acceptable salt or solvate thereof, wherein n is 2.

32. A compound selected from: 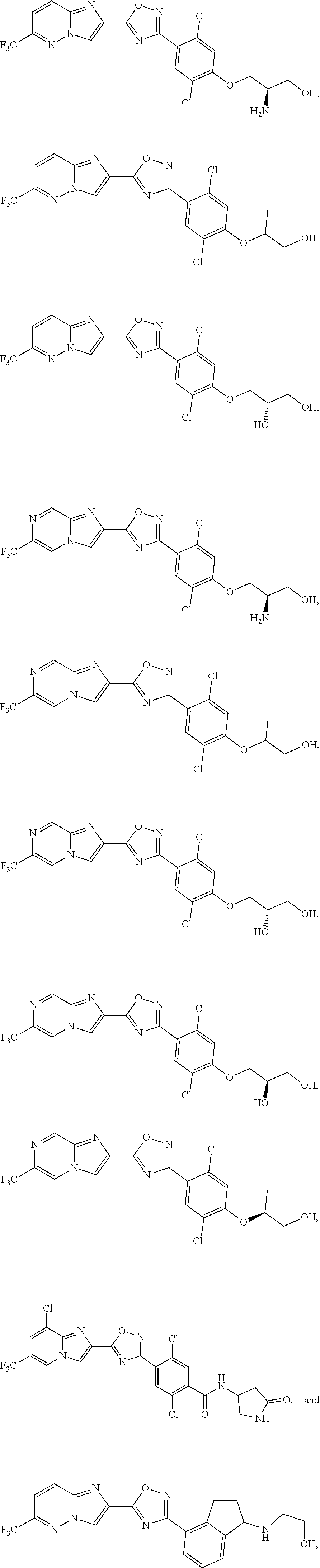
or a pharmaceutically acceptable salt, or pharmaceutically acceptable solvate thereof.

33. A pharmaceutical composition comprising a pharmaceutically acceptable diluent, excipient or binder, and a compound of any one of claims 1-32; or a pharmaceutically acceptable salt or solvate thereof.

34. A method of modulating sphingosine-1-phosphate (S1P) receptor activity comprising contacting the S1P receptor, or portion thereof, with a compound, or a pharmaceutically acceptable salt or solvate thereof, according to any one of claims 1-32.

35. A method of treating a disease, disorder or condition in a mammal that would benefit from sphingosine-1-phosphate (S1P) receptor modulation comprising administering to the mammal a therapeutically effective amount of a compound, or a pharmaceutically acceptable salt or solvate thereof, according to any one of claims 1-32.

36. The method of claim 35, wherein the disease, disorder or condition in a mammal is selected from multiple sclerosis, ulcerative colitis, and Crohn's disease.
